(19) United States
(12) Patent Application Publication
(10) Pub. No.: US 2021/0268098 A1
(43) Pub. Date: Sep. 2, 2021

(54) METHODS AND COMPOSITIONS FOR ALPHAVIRUS VACCINE

(71) Applicant: UAB Research Foundation, Birmingham, AL (US)

(72) Inventors: Elena I. Frolova, Birmingham, AL (US); IIya V. Frolov, Birmingham, AL (US)

(21) Appl. No.: 17/264,377

(22) PCT Filed: Aug. 2, 2019
(86) PCT No.: PCT/US2019/044791

§ 371 Date: Jan. 29, 2021

Related U.S. Application Data
(60) Provisional application No. 62/714,598, filed on Aug. 3, 2018.

(51) Int. Cl. A61K 39/12; C07K 14/18; C12N 15/86; A61P 31/14

(52) U.S. Cl. CPC A61K 39/12; C07K 14/1808; A61K 2039/5254; A61P 31/14; C12N 15/86

(57) ABSTRACT

The present invention provides an attenuated Old World alphavirus particle and methods of making same and using same as a vaccine and in gene therapy and immunotherapy methods.

### STATEMENT OF PRIORITY

This application claims the benefit, under 35 U.S.C. § 119(e), of U.S. Provisional Application No. 62/714,598 filed on Aug. 3, 2018, and the entire contents of which are incorporated by reference herein.

### STATEMENT OF GOVERNMENT SUPPORT

This invention was made with government support under Grant Nos. AI073301 and AI133159 awarded by the National Institutes of Health. The government has certain rights in the invention.

### FIELD OF THE INVENTION

The present invention relates generally to the field of virology and vaccine development. More specifically, the present invention provides a method to attenuate Old World alphaviruses for use as vaccines.

### BACKGROUND OF THE INVENTION

Alphaviruses that circulate in the Old World (the OW alphaviruses) include a wide variety of human and animal pathogens. Within recent years, chikungunya virus (CHIKV) became a viral pathogen of particular importance, because it has already moved to the western hemisphere and became adapted for transmission by new species of mosquitoes that are prevalent in the US. It induces highly debilitating disease characterized by excruciating joint pain and severe, persistent polyarthritis. To date, no safe and efficient vaccines or therapeutic means have been developed against CHIKV and any other OW alphaviruses.

High pathogenicity of CHIKV and related OW alphaviruses is determined by their ability to efficiently invade host immunity by inhibiting an antiviral response. Their nonstructural protein 2, nsP2, mediates degradation of the catalytic subunit of cellular DNA dependent RNA polymerase II (RPB1). This in turn leads to rapid inhibition of cellular transcription and makes cells unable to mount an antiviral response that can inhibit or prevent spread of the infection on cellular and organismal levels.

The present invention overcomes previous shortcomings in the art by providing an attenuated OW alphavirus and methods of its use as a vaccine and a viral vector.

### SUMMARY OF THE INVENTION

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In one embodiment, the present invention provides an alphavirus nsP2 protein comprising one or more amino acid substitutions that disrupt the ability of nsP2 to induce RPB1 degradation and inhibition of cellular transcription, comprising at least a substitution at: a) amino acid 674 in chikungunya virus (CHIKV); b) amino acid 675 in CHIKV; c) amino acid 676 in CHIKV; and/or d) amino acid 677 in CHIKV, or at the corresponding amino acid positions in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses.

In a further embodiment, the present invention provides an attenuated alphavirus particle comprising a nucleic acid molecule encoding an nsP2 protein that comprises a substitution as described herein, for use as a vaccine.

In an additional embodiment, the present invention provides a recombinant replicon nucleic acid, comprising: a) the nucleotide sequence of a 5′ terminus of alphavirus genome that is required for genome translation and replication; b) a nucleotide sequence encoding alphavirus nonstructural proteins nsP1, nsP3, nsP4 and nsP2, wherein said nsP2 comprises one or more amino acid substitutions, comprising at least substitutions at: a) amino acid 674 in chikungunya virus (CHIKV); b) amino acid 675 in CHIKV; c) amino acid 676 in CHIKV; and/or d) amino acid 677 in CHIKV, or at the corresponding amino acid positions in Sindbis virus (SINV), Aura virus (AURV), Mayaro virus (MAYV), Ross River virus (RRV), Semliki Forest virus (SFV), Getah virus (GETV), or O' Nyong Nyong virus (ONNV); c) at least one alphavirus subgenomic promoter; d) at least one heterologous nucleic acid molecule; and e) a nucleotide sequence encoding a 3′ terminus of alphavirus genome that functions in regulation of viral genome replication. Also provided is an infectious alphavirus particle comprising the recombinant replicon nucleic acid of this invention.

Additionally, the present invention provides a method of making infectious alphavirus particles, comprising introducing the recombinant replicon nucleic acid of this invention into a helper cell, packaging cell or producer cell under conditions whereby infectious alphavirus particles are produced in the cell.

Furthermore, the present invention provides a method of producing a protein of interest in a cell, comprising introducing into the cell the recombinant replicon nucleic acid of this invention, wherein the recombinant replicon nucleic acid comprises a nucleotide sequence encoding the protein of interest, under conditions whereby the recombinant replicon nucleic acid is expressed and the protein of interest is produced.

The present invention also provides a method of inducing and/or enhancing an immune response in a subject, comprising administering to the subject an effective amount of the attenuated alphavirus particle of this invention, the recombinant replicon nucleic acid of this invention, the vector of this invention, the cell of this invention, and/or the infectious alphavirus particle of this invention, thereby inducing and/or enhancing an immune response in the subject as compared with a control subject.

In additional embodiments, the present invention provides a method of treating and/or preventing an alphavirus infection and/or treating the effects of an alphavirus infection in a subject, comprising administering to the subject an immunogenic amount of the attenuated alphavirus particle of this invention, the recombinant replicon nucleic acid of this invention, the vector of this invention, the cell of this invention, and/or the infectious alphavirus particle of this invention, thereby treating and/or preventing an alphavirus infection in the subject and/or treating the effects of an alphavirus infection in the subject.

In a further embodiment, the present invention provides a method of screening test agents and compounds for anti-alphavirus activity, comprising generating a cell line in which the recombinant replicon nucleic acid of this invention, encoding a marker protein such as green fluorescent protein (GFP) or luciferase, is persistently replicated; introducing into cells of this cell line a test agent or compound; and observing the effect of the presence of the test agent or compound on expression of the marker protein in the cell to evaluate the effect of the test agent or compound on the ability of the recombinant replicon nucleic acid to replicate, thereby identifying a test agent or compound that inhibits or enhances recombinant replicon nucleic acid replication.

An additional embodiment is a method of attenuating an alphavirus, comprising substituting one or more than one amino acid residue in the variable (V) region of the nonstructural protein 2 (nsP2) of the alphavirus, wherein the one or more amino acid residues that are substituted are amino acids 674, 675, 677 and/or 678 of CHIKV or the corresponding amino acid residues in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses. Also provided herein is an attenuated alphavirus particle, produced by this method, as well as a vaccine formulation comprising the attenuated alphavirus particle of this invention in a vaccine diluent.

### BRIEF DESCRIPTION OF THE DRAWINGS

FIGS. 1A-1D: Adaptive mutations accumulate in discrete regions of SINV nsP2. (A) Schematic presentations of VEE replicon encoding SINV nsP2-GFP protein and the selection of noncytopathic SINV nsP2. The N-terminus of nsP2-GFP is fused with ubiquitin (Ubi) to mediate formation of the natural first amino acid. The Pac gene is cloned under control of another subgenomic promoter. Cells were electroporated with the in vitro-synthesized replicon RNA and then treated with puromycin. Colonies of GFP-positive, Pur^{R} cells were selected for further analysis. (B) The list of mutations identified in the SINV nsP2 gene of replicons in GFP-positive cells. Mutations that did not affect nuclear localization of SINV nsP2-GFP are depicted in red. Mutations that led to predominantly cytoplasmic localization of nsP2 are depicted in black. Mutations that prevent nsP2 import into the nucleus and are exposed on the protein surface are depicted in blue. (C) Location of SINV nsP2 mutations identified in this and our prior studies. (D) Positions of the identified mutations on the 3D model of the SINV nsP2 protease domain.

FIGS. 2A-2C: Mutations that affect translocation of SINV nsP2-GFP to the nucleus are lethal for virus replication, but do not affect its ability to induce RPB1 cleavage. (A) Virus replication rates, RNA infectivity and infectious virus titers of the designed SINV nsP2 mutants upon transfection of the in vitro-synthesized RNA into BHK-21 cells. (B) BHK-21 were infected with packaged VEEV replicons encoding different variants of Ubi-nsP2-GFP and analyzed by confocal microscopy at 6 h PI. Images are presented as multiple image projections of a 1 μm x-y section (6 optical sections) through the nuclei. Scale bars: 10 μm. (C) Western blot analysis of cells infected with VEE replicons expressing wt or mutant nsP2-GFP proteins with or without nuclear localization signal.

FIGS. 3A-3C: In the context of SINV, mutation P683Q of nsP2 does not make the virus noncytopathic, despite its inability to induce degradation of RPB1. (A) Schematic presentations of wt and mutant viruses. (B) BHK-21 cells were infected with indicated viruses at an MOI of 20. Cell lysates were prepared at the indicated times PI, and amount of RPB1 and nsP2 were analyzed by Western blot. (C) BHK-21 and NIH 3T3 cells were infected with the indicated viruses at MOIs of 10 and 20, respectively. At the indicated times PI, media were replaced and virus titers were measured by plaque assay on BHK-21 cells.

FIGS. 4A-4D: SINV P683 nsP2 mutants are incapable of inducing RPB1 degradation and efficiently induce IFN-β response. (A) Schematic presentations of wt and mutant viruses. (B) NIH 3T3 cells were infected with the indicated SINV variants at an MOI of 20. Cell lysates were prepared at 8 h PI. The integrity of RPB1 and accumulation of SINV nsP2 were analyzed by Western blot using specific Abs. (C) and (D) NIH 3T3 cells were infected with indicated viruses at an MOI of 20. Virus titers (C) and concentration of IFN-β in the media (D) were assessed at 16 h PI as described herein. Data are shown as mean±SD of 3 biological repeats.

FIGS. 5A-5C: Combination of mutations in nsP2 and other nsPs of SINV replicons make them dramatically less cytopathic. (A) The schematic presentation of SINV replicons and their ability to induce formation of Pur^{r} BHK-21 cell colonies. BHK-21 cells were electroporated with the in vitro-synthesized replicon RNAs, and puromycin selection was applied at 24 h PI. Cell colonies were stained with Crystal violet at 7 days post transfection. Images present the plates seeded with equal numbers of electroporated cells. (B) BHK-21 cells were electroporated with the in vitro-synthesized RNAs of the indicated replicons (SEQ ID NOS:11-12). The puromycin selection was applied at 24 h PI, and cell colonies were stained with Crystal violet at 7 days post transfection. Images present the plates seeded with equal numbers of electroporated cells. (C) BHK-21 cells were electroporated with equal amounts of the in vitro-synthesized RNAs of the indicated replicons. Cell lysates were prepared at 24 h post electroporation and analyzed using nsP2-, nsP3-, GFP- and tubulin-specific Abs. PEP—post electroporation.

FIGS. 6A-6D: Defined mutations in nsP2 and nsP3 make SINV capable of noncytopathic replication in vertebrate cells without a strong effect on virus replication rates. (A) Schematic presentation of virus mutants, infectivity of the in vitro-synthesized RNAs in the infectious center assay on BHK-21 cells and virus titers at 24 h post electroporation. (B) NIH 3T3 cells were infected with the indicated variants at an MOI of 20. At 8 h PI, media were harvested and virus titers and INF-β were assessed as described herein. Data are shown as mean±SD of 3 biological repeats. (C) NIH 3T3 cells were infected with the indicated variants at an MOI of 20. Cell lysates were prepared at 8 h PI and analyzed by using RPB1-, nsP2-, STAT1-, pSTAT1- and tubulin-specific Abs. (D) NIH 3T3 cells and their Mavs KO derivatives were infected with the indicated variants at an MOI of 20. Media were replaced every 24 h and virus titers were measured by plaque assay on BHK-21 cells. LOD—limit of detection, PEP—post electroporation.

FIGS. 7A-7B: The identified nsP2- and nsP3-specific mutations make SINV incapable of inducing transcriptional and translational shutoffs, respectively. (A) NIH 3T3 cells were infected with the indicated viruses at an MOI of 20. RNAs were metabolically labeled with [³H]uridine (20 mCi/ml) between 3 and 7 h PI in the absence of ActD. RNAs were isolated and analyzed by agarose gel electrophoresis as described herein. (B) NIH 3T3 cells were infected with the indicated viruses at an MOI of 20. At 6 h PI, proteins were metabolically labeled with [³⁵S]methionine for 30 min and analyzed on a sodium dodecyl sulfate-10% polyacrylamide gel. The gels were dried and autoradiographed.

FIGS. 8A-8E: Defined mutation in the catalytic center of SINV nsP3 does not affect development of transcriptional shutoff. (A) The schematic presentation of the genomes of SINV variants with mutated nsP2 and nsP3. (B) NIH 3T3 cells were infected with the indicated viruses at an MOI of 20. Virus titers were assessed at 8 h PI by plaque assay on BHK-21 cells. (C) NIH 3T3 cells were infected with the indicated viruses at an MOI of 20. Concentration of the released IFN-β was measured at 18 h PI. (D) NIH 3T3 cells were infected with the indicated viruses at an MOI of 20. Cell lysates were prepared at 8 h PI and analyzed by Western blot using RPB1-, nsP2- and tubulin-specific Abs. Quantitative analysis of RPB1 concentration was performed on a LI-COR imager. (E) NIH 3T3 cells and their Mavs KO derivatives were infected with the indicated variants at an MOI of 20. Media were replaced every 24 h and virus titers were measured by plaque assay on BHK-21 cells. Data in panels B and C are shown as mean+SD of 3 biological repeats.

FIGS. 9A-9B: CHIKV nsP2-specific V peptide demonstrates high variability and is located on the surface of the SOM domain. (A) The nsP2 protein sequences of 365 OW alphaviruses were downloaded from Virus Pathogen Resources, ViPR. The sequences were aligned using Muscle in Jalview. The aligned fragments of nsP2 corresponding aa 679-688 of SINV were copied, and redundant sequences and sequences presented by a single strain were deleted. The remained sequences were re-aligned (SEQ ID NOS:13-24). Mutations identified in attenuated SINV are shown on top of the alignment. (B) The 3D structures of CHIKV (3TRK) and SINV (modeled based on 4GUA) were superimposed in Discovery Studio Visualizer using sequence alignment. The structure of CHIKV nsP2 is presented as a light magenta solid ribbon. The structure of SINV nsP2 is presented as a light turquoise solid ribbon. The P726 (SINV) and P718 (CHIKV) presented as sticks. The CHIKV-specific ATLG fragment is colored in blue, and SINV-specific PQA fragment is colored in red. Virus name abbreviations: AURV-Aura virus, CHIKV-chikungunya virus, GETV-Getah virus, MAYV-Mayaro virus, RRV-Ross River virus, ONNV-O' Nyong Nyong virus, SFV-Semliki Forest virus, SINV-Sindbis virus.

FIGS. 10A-10F: SINV-specific mutations in CHIKV-specific V peptide differentially affect viral replication rates and activation of the antiviral response. (A) The schematic presentation of recombinant CHIKV genomes encoding indicated mutations in V peptide. (B) NIH 3T3 cells were infected with the indicated viruses at an MOI of 20 PFU/cell. Media were replaced at the indicated times PI, and viral titers were determined by plaque assay in BHK-21 cells. (C) and (D) NIH 3T3 cells were infected with wild-type and mutant viruses at an MOI of 20 PFU/cell. Viruses were harvested at 18 h PI. Infectious titers and concentrations of the released IFN-beta were determined in the same samples as described herein. The significance of differences in titers was estimated by one-way ANOVA (n=3). (E) Western blot analysis of RPB1 levels and ns polyprotein processing in CHIKV-infected cells. NIH 3T3 cells were infected with the indicated viruses at an MOI of 20 PFU/cell, harvested at 8 h PI, and the lysates were analyzed by Western blot for the levels of RPB1 and for the ns polyprotein processing. Membranes were scanned on Odyssey imager (LI-COR). (F) BHK-21 and NIH 3T3 cells were infected with CHIKV/V3/GFP at an MOI of 10 PFU/cell. They were incubated for 10 days, and either media were changed every 24 h, or cells were also split upon reaching confluency. Viral titers were determined by plaque assay on BHK-21 cells.

FIGS. 11A-11B: The QQA mutation in CHIKV nsP2 V peptide impairs P12 processing. (A) The schematic presentation of recombinant CHIKV genomes with indicated mutation V peptide and mutated nsP2/3 cleavage site. (B) NIH 3T3 cells were infected with the indicated viruses at an MOI of 20 PFU/cell and harvested at 8 h PI. Presence of individual nsPs in cell lysates and incompletely cleaved polyproteins were analyzed by Western blots using custom antibodies against CHIKV nsP1, nsP2, and nsP3. Membranes were analyzed on Odyssey imager (LI-COR).

FIGS. 12A-12C: Defined V peptides in nsP2 make CHIKV replicon, CHIKrep/Pac, noncytopathic for BHK-21 cells. (A) The schematic presentations of CHIKV replicon with cloned library of V peptides in nsP2 protein and the scheme of selection of noncytopathic replicons. Cells were electroporated with the in vitro-synthesized replicon RNA and then treated with puromycin to select colonies of Pur^{R} cells. (B) The aa sequences of V peptide and detected additional mutations in CHIKV nsP2 found in the noncytopathic replicons of primarily selected clones of Pur^{R} cells. (C) The aa sequences of V peptide and detected additional mutations in CHIKV nsP2 found in the noncytopathic replicons after 3-weeks-long passaging of the pool of Pur^{R} cells (see the text for details). In (B) and (C), aa sequences used in further experiments are indicated in red.

FIGS. 13A-13B: Replacement of V peptide in CHIKrep/GFP/Pac by selected aa sequences makes replicon noncytopathic and capable of persistent replication. (A) The schematic presentation of the designed replicons containing indicated mutations in V peptide and additional mutation in nsP2 (in CHIKrep/RLH,A730V/GFP/Pac), and their efficiency in the formation of colonies of Pur^{R}, GFP-positive cells upon electroporation of the in vitro-synthesized RNAs. (B) BHK-21 cells were electroporated with equal amounts of the in vitro-synthesized replicon RNAs and harvested either at 8 h post transfection (CHIKV/GFP RNA-transfected cells) or 3-7 days post-transfection and puromycin selection of cells with mutant replicons. Equal numbers of cells were used for analysis. Levels of indicated nsPs and GFP in cell lysates were evaluated by Western blots using specific Abs. Membranes were scanned on Odyssey imager (LI-COR).

FIGS. 14A-14D: CHIKV variants containing selected mutations in V peptide efficiently replicate and are highly potent IFN-β inducers. (A) The schematic presentation of recombinant CHIKV genomes containing indicated mutations in V peptide and additional mutation in nsP2 (CHIKV/RLH,A730V/GFP), RNA infectivity in the infectious center assay and infectious titers in the stocks harvested at 24 h post electroporation of BHK-21 cells. (B) and (C) NIH 3T3 cells were infected with the indicated viruses at an MOI of 50 PFU/cell, and samples were harvested at 22 h PI. Viral titers were determined by plaque assay on BHK-21 cells, and concentrations of IFN-beta were assessed by ELISA as described herein. (D) NIH 3T3 cells were infected with the indicated viruses and an MOI of 20 PFU/cell and harvested at 9 h PI. The levels of degradation of RPB1 and levels of nsP2 were determined by Western blot using specific Abs. Membranes were analyzed on Odyssey imager (LI-COR). These experiments were reproducibly repeated more than three times, and the results of one of the representative experiments are presented.

FIGS. 15A-15B: Mutations in V peptide strongly affect CHIKV infection spread in type I IFN-competent cells. (A) Monolayers of NIH 3T3 cells were infected with indicated variants and then covered by agarose-containing media supplemented with 3% FCS. At 24 h PI, cells were fixed by 4% PFA and imaged on Cytation 5 Cell Imaging Multi-Mode Reader (BioTek). GFP-positive foci are presented. (B) Average GFP intensity per cell was estimated for above-presented images using Gen5 software (BioTek).

FIG. 16: Mutations in V peptide do not affect nuclear localization of CHIKV nsP2. NIH 3T3 cells were infected with indicated recombinant viruses, and at 6 h PI, they were fixed with 4% paraformaldehyde, permeabilized and stained with nsP2-specific Abs and fluorescent secondary Abs. Images were acquired on confocal Zeiss 710 microscope.

FIG. 17: The CHIKV nsP2 mutants are cleared without cytopathic effect from NIH 3T3 cells and persistently replicate in MAVS KO NIH 3T3 cells. NIH 3T3 and MAVS KO NIH 3T3 cells were infected with the indicated viruses at an MOI of 20 PFU/cell. Media were replaced every 24 h, and cells were split upon reaching confluency. Viral titers were determined by plaque assay on BHK-21 cells, and the samples harvested from infected NIH 3T3 cells were used for assessment of IFN-beta concentration as described herein. Images were taken on fluorescence microscope at day 9 PI of MAVS KO NIH 3T3 cells to demonstrate that all of the cells remained GFP-positive, and thus, contained persistently replicating viruses.

FIGS. 18A-18B: The designed CHIKV mutants do not induce transcriptional shutoff in the infected cells but downregulate translation of cellular mRNAs. (A) NIH 3T3 cells were infected with indicated viruses at an MOI of 20 PFU/cell. RNAs were metabolically labeled with [³H]uridine in the absence of ActD between 4 and 8 h PI. They were analyzed by agarose gel electrophoresis in denaturing conditions as described herein. (B) NIH 3T3 cells were infected at an MOI of 20 PFU/cell, and at 6 h PI, proteins were metabolically labeled for 30 min with [³⁵S]methionine and analyzed by SDS-PAGE as described herein.

FIGS. 19A-19B: V peptide in both CHIKV and SFV nsP2 plays critical roles in proteins' function in RPB1 degradation. (A) The schematic presentation of VEEV replicons expressing different forms of SINV, CHIKV and SFV nsP2. (B) NIH 3T3 cells were infected with indicated packaged replicons at an MOI of 20 inf.u/cell. At 8 h PI, cells were harvested and cell lysates were analyzed by Western blot using RPB1-, GFP- and alpha-tubulin-specific Abs. Membranes were scanned on Odyssey imager (LI-COR) and processed using the manufacturer's software. The experiment was repeated twice with reproducible results.

FIGS. 20A-20B: CHIKV 181/25 derivatives with mutated VLoop are viable. (A) The alignment of CHIKV nsP2 fragment containing indicated mutations (SEQ ID NOS:25-28). Dashes indicate identical aa. The numbers correspond to aa in nsP2 of parental CHIKV 181/25. (B) RNA infectivity and viral titers at 24 h post electroporation. BHK-21 cells were electroporated with 3 mg of in vitro-synthesized RNAs of parental CHIKV 181/25 and the designed mutants. Electroporated cells were used for the infectious center assay and for generating viral stocks. Viral titers were assessed by plaque assay on BHK-21 cells. Transfection experiments were reproducibly repeated three times and the presented values are ranges from three individual experiments.

FIG. 21: CHIKV nsP2 mutants efficiently replicate in rodent and human cells. 5×10⁵ BHK-21, Vero, NIH 3T3, HEK 293, MRC-5 and BJ-5ta cells in 6-well Costar plates were infected with CHIKV 181/25 or designed mutants at an MOI of 0.01 PFU/cell. At the indicated time points, media were replaced, and viral titers were determined by plaque assay on BHK-21 cells. The experiments were reproducibly repeated 3 times, and the results of one of the representative experiments are presented.

FIGS. 22A-22B. The designed CHIKV nsP2 mutants demonstrate efficient synthesis of virus-specific RNAs and viral structural proteins. (A) 5×10⁵ cells in the 6-well Costar plate were infected with parental CHIKV 181/25 and designed mutants at an MOI of 20 PFU/cell. At 7 h PI, they were washed with PBS, and proteins were metabolically labeled for 30 min with [³⁵S]methionine. Equal amounts of lysates were analyzed by gel electrophoresis in 10% NuPAGE gels (Invitrogen), followed by autoradiography. (B) 5×10⁵ cells in the 6-well Costar plate were infected with the indicated viruses at an MOI of 20 PFU/cell. Virus-specific RNAs were metabolically labeled between 4 and 8 h PI in complete media, supplemented with [³H]uridine (20 mCi/ml) and Actinomycin D (1 mg/ml), then RNAs were isolated and analyzed by agarose gel electrophoresis.

FIGS. 23A-23C. CHIKV nsP2 mutants, but not the parental CHIKV 181/25, are potent inducers of IFN-b in both murine and human cell lines. NIH 3T3 (A), MRC-5 (B), and HFF-1 (C) cells were infected at an MOI of 20 PFU/cell with nsP2 mutants and parental CHIKV 181/25. At 18 h PI, the supernatants were harvested to determine viral titers and the levels of IFN-β. n.d. indicates that the concentration of IFN-β was below the limit of detection. Similar experiments were made multiple times with reproducible results. The results of one of the representative experiments are presented.

FIG. 24. CHIKV nsP2 mutants do not form plaques on the cells competent in type I IFN induction and signaling. CHIKV nsP2 mutants and parental CHIKV 181/25 were used in plaque assay performed on the indicated cell lines. This figure displays wells with monolayers of different cells, which were infected with the same dilutions of the indicated viruses. All of the plates were stained with crystal violet after 3 days of incubation at 37° C.

FIG. 25. Infections of murine NIH 3T3 and human MRC-5 fibroblasts with CHIKV nsP2 mutants result in ISG activation. NIH 3T3 and MRC-5 cells were infected with the indicated viruses at an MOI of 20 PFU/cell, and, at 16 h PI, the induction of indicated ISGs and IFN-b was evaluated by RT-qPCR. The fold increase in the ISGs transcript level in virus-infected cells relative to the mock-infected cells were calculated using ΔΔCT method. The experiment was reproducibly repeated three times with similar results, and the data from one representative experiment are shown. Average values from triplicate samples are presented, but the error bars are too small to be visible at this scale.

FIGS. 26A-26B. The designed CHIKV 181/25-based nsP2 mutants remain immunogenic and protect mice against following challenge with wCHIKV. Two-to-three-week old C57BL/6 mice (n=6/group) were infected in the left foot pad with 5×10³ PFU of CHIKV 181/25 and its indicated nsP2 mutants. (A) The levels of neutralizing Abs were assessed at day 21 PI. (B) Mice were challenged on day 25 PI with 10⁵ PFU of wCHIKV using the same route of infection. Blood samples were collected on days 1, 2, and 3, and levels of viremia were assessed by plaque assay on BHK-21 cells. Each data point represents a value from an individual mouse. The dashed line indicates the limit of detection (LOD) in plaque assay.

FIGS. 27A-27B. Mutations in nsP2 of wCHIKV strongly affect the development of viremia in mice. (A) Two-to-three-week old C57BL/6 mice (n=6/group) were infected in the left foot pad with 5×10³ PFU of wCHIKV nsP2 mutants and parental wCHIKV. Blood samples were taken an days 1, 2 and 3 PI, and the levels of viremia were assessed by plaque assay on BHK-21 cells. The dashed line indicates the limit of detection (LOD). (B) Weight change post infection with indicated viruses.

FIGS. 28A-28B. Additional mutations in the macro domain of nsP3 in CHIKV/NGK/GFP reduce viral cytopathogenicity in human and Vero cells. (A) The schematic presentation of the genomes of recombinant viruses. (B) 5×10⁵ cells of the indicated cell lines in 6-well Costar plates were infected with the mutants at an MOI of 20 PFU/cell. Media were replaced at the indicated time points, and cells were split upon reaching confluency. Viral titers were determined by plaque assay on BHK-21 cells. The dashed line indicates the limit of detection (LOD).

FIGS. 29A-29B. Mutations in the macro domain of nsP3 in wCHIKV/NGK variant have no negative effect on viral replication in mice. (A) The schematic presentation of the recombinant genomes. (B) Two-to-three-week old C57BL/6 mice (n=6/group) were infected in the left foot pad with 5×10³ PFU of the indicated mutants and parental wCHIKV. Blood samples were collected on days 1, 2, and 3 PI to analyze the levels of viremia. The dashed line indicates the limit of detection (LOD). (C) Titers of neutralizing antibodies were evaluated on day 21 PI.

### DETAILED DESCRIPTION OF THE INVENTION

The present invention will now be described more fully hereinafter with reference to the accompanying drawings and specification, in which preferred embodiments of the invention are shown. This invention may, however, be embodied in different forms and should not be construed as limited to the embodiments set forth herein.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

As used herein, “a,” “an” or “the” can mean one or more than one. For example, “a” cell can mean a single cell or a multiplicity of cells.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of dose (e.g., an amount of a non-viral vector) and the like, is meant to encompass variations of ±20%, ±10%, ±5%, ±1%, ±0.5%, or even ±0.1% of the specified amount.

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461, 463 (CCPA 1976) (emphasis in the original); see also MPEP § 2111.03. Thus, the term “consisting essentially of” when used in a claim of this invention is not intended to be interpreted to be equivalent to “comprising.”

Alphaviruses are a group of human and animal pathogens, which are widely distributed all over the world. They replicate in the cytoplasm of the infected cells, and this replication does not depend on nuclei. Alphavirus genomes are represented by single-stranded RNA molecule of positive polarity of almost 11.5 kb. This RNA mimics the structure of cellular messenger RNAs, in that it contains a cap structure at the 5′ terminus, and a poly(A) sequence at the 3′ terminus. Upon delivery into the cells, the viral genome is translated into the polyprotein precursor of the nonstructural proteins. The latter polyprotein is sequentially self-processed by the encoded protease, and these nonstructural proteins nsP1, nsP2, nsP3 and nsP4 form the replicative enzyme complex, which amplifies the viral genome and synthesizes additional subgenomic RNA that is a template for synthesis of viral structural proteins. These structural proteins (capsid, E2 and E1) ultimately form viral particles.

Besides being involved in RNA replication, the Old World (OW) alphavirus nsP2 protein is transported to the nucleus, where it induces rapid degradation of the catalytic subunit of DNA dependent RNA polymerase II, RPB1. This leads to global transcriptional shutoff, inhibition of antiviral response and cell death within 24 h post infection.

The present invention is based on the identification of a peptide in the nsP2 protein of the OW alphaviruses that determines the ability of the nsP2 protein i) to induce degradation of the catalytic subunit of the cellular DNA dependent RNA polymerase II, ii) to inhibit cellular transcription, and iii) to cause cytopathic effect. Specific mutations in this peptide result in virus attenuation and make it unable to spread among the cells having no defects in type I IFN response and signaling. These mutations also strongly affect the ability of alphavirus-based expression systems to induce cell death and cytopathic effect. The method for selecting attenuating mutations is fully developed, as described herein, and can be applied to other pathogenic OW alphaviruses for designing highly attenuated viral mutants and noncytopathic replicons.

In this invention, we have identified a short aa sequence, a variable (V) peptide, in the OW alphavirus nsP2 that plays a critical role in the ability of this protein to inhibit cellular transcription. The important role of this peptide in inhibition of cellular transcription was first suggested in experiments with Sindbis virus. Single point mutations resulting in substitution of the proline residue by other amino acids did not change high replication rates of Sindbis virus, but made it a strong type I IFN inducer. Thus, they affected the ability of the virus to inhibit cellular transcription.

Thus, the present invention is based on the unexpected discovery of a variable (V) region in an Old World alphavirus nonstructural protein 2 (nsP2) that can be mutated (e.g., by substitution), resulting in an alphavirus nsP2 protein that is disrupted in the ability to induce RPB1 degradation and inhibition of cellular transcription in an infected cell. These mutations in the nsP2 alphavirus protein allows for production of an attenuated alphavirus particle that can be used as a vaccine. These attenuated particles are very strong type I interferon inducers, and there is no effect on the replication rate of these particles in infected cells. This allows for large-scale production without biocontainment conditions.

Thus, in one embodiment, the present invention provides an alphavirus nsP2 protein comprising one or more amino acid substitutions that disrupts the ability of nsP2 to induce RPB1 degradation and inhibition of cellular transcription, comprising at least a substitution at: a) amino acid 674 in chikungunya virus (CHIKV); b) amino acid 675 in CHIKV; c) amino acid 676 in CHIKV; and/or d) amino acid 677 in CHIKV, or at the corresponding amino acid positions in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses.

In some embodiments, the alphavirus nsP2 protein is from CHIKV and amino acid residues A674, T675 and L677 are substituted with an amino acid other than wild type. Nonlimiting examples of specific substitutions at 674ATL676 include ERR, FFR, RSR, NGK, DID, RLH, MLR, VRR, SGV, RLE, RVP, KLN, QMS, HIK, FIH, LFD, EMS, IKW or YMS.

In some embodiments, the alphavirus nsP2 protein is from SINV and amino acid residues P683 and/or Q684 are substituted with an amino acid other than wild type. Nonlimiting examples of specific substitutions at P683 and/or Q684 include P683Q, P683E, P683N, P683S and/or Q684P.

In some embodiments, the alphavirus nsP2 protein is SFV and amino acid residues A674, D675, A676 and/or G677 are substituted with an amino acid other than wild type. Nonlimiting examples of specific substitutions at 674ADA676 include NGK or RLE.

In addition, the present invention provides a method of attenuating an alphavirus, comprising substituting one or more than one amino acid residue in the variable (V) region of the nonstructural protein 2 (nsP2) of the alphavirus, wherein the one or more amino acid residues that are substituted are amino acids 674, 675, 677 and/or 678 of CHIKV or the corresponding amino acid residues in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses.

The present invention also provides an attenuated alphavirus particle comprising a nucleic acid molecule encoding the alphavirus nsP2 protein of this invention, which can be attenuated alphavirus particle produced by the above method. The attenuated alphavirus particle of this invention can be present in a pharmaceutical composition, which can be an immunogenic composition, comprising a pharmaceutically acceptable carrier. The attenuated alphavirus particle of this invention can be present in a vaccine formulation, comprising a vaccine diluent, (e.g., a vaccine diluent as would be known in the art).

The present invention also provides a recombinant replicon nucleic acid, comprising: a) the nucleotide sequence of a 5′ terminus of alphavirus genome that is required for genome translation and replication; b) a nucleotide sequence encoding alphavirus nonstructural proteins nsP1, nsP3, nsP4 and nsP2, wherein said nsP2 comprises one or more amino acid substitutions, comprising at least substitutions at: a) amino acid 674 in chikungunya virus (CHIKV); b) amino acid 675 in CHIKV; c) amino acid 676 in CHIKV; and/or d) amino acid 677 in CHIKV, or at the corresponding amino acid positions in Sindbis virus (SINV), Aura virus (AURV), Mayaro virus (MAYV), Ross River virus (RRV), Semliki Forest virus (SFV), Getah virus (GETV), or O' Nyong Nyong virus (ONNV); c) at least one alphavirus subgenomic promoter; d) at least one heterologous nucleic acid molecule; and e) a nucleotide sequence encoding a 3′ terminus of alphavirus genome that functions in regulation of viral genome replication.

In some embodiments, the recombinant replicon nucleic acid of this invention can be DNA and in some embodiments the recombinant replicon nucleic acid of this invention can be RNA. Also provided herein is a complementary DNA (cDNA) molecule encoding the recombinant replicon nucleic acid in RNA form. Further provided herein is an alphavirus vector comprising a 5′ promoter operably linked to the cDNA of this invention. A cell comprising said vector is further provided in this invention.

In some embodiments of a nucleic acid construct of this invention, a promoter for directing transcription of RNA from DNA, i.e., a DNA dependent RNA polymerase, can be employed. In the RNA replicon nucleic acid embodiments of this invention, the promoter is utilized to synthesize RNA in an in vitro transcription reaction, and specific promoters suitable for this use include, but are not limited to, the SP6, T7, and T3 RNA polymerase promoters. In the DNA replicon nucleic acid embodiments, the promoter functions within a cell to direct transcription of RNA. Potential promoters for in vivo transcription of the construct include, but are not limited to, eukaryotic promoters such as RNA polymerase II promoters, RNA polymerase I and RNA polymerase III promoters, and/or viral promoters such as MMTV and MoSV LTR, SV40 early region, RSV or CMV or β-actin promoter. Many other suitable mammalian and viral promoters for the present invention are available and are known in the art. Alternatively, DNA dependent RNA polymerase promoters from bacteria or bacteriophage, e.g., SP6, T7, and T3, can be employed for use in vivo, with the matching RNA polymerase being provided to the cell, either via a separate plasmid, RNA vector, or viral vector.

In a particular embodiment, the matching RNA polymerase can be stably transformed into a helper cell line under the control of an inducible or continuous promoter. Constructs that function within a cell can function as autonomous plasmids transfected into the cell and/or they can be stably transformed into the genome. In a stably transformed cell line, the promoter can be an inducible promoter, so that the cell will only produce the RNA polymerase encoded by the stably transformed construct when the cell is exposed to the appropriate stimulus (inducer). Helper constructs as described herein are introduced into the stably transformed cell concomitantly with, prior to, and/or after exposure to, the inducer, thereby effecting expression of the alphavirus structural proteins. Alternatively, constructs designed to function within a cell can be introduced into the cell via a viral vector, such as, e.g., adenovirus, poxvirus, adeno-associated virus, SV40, retrovirus, nodavirus, picornavirus, vesicular stomatitis virus, and baculoviruses with mammalian pol II promoters.

As used herein, an “alphavirus subgenomic promoter” or “26S promoter” is a promoter as originally defined in a wild type alphavirus genome that directs transcription of a subgenomic messenger RNA as part of the alphavirus replication process. Such a promoter can have a wild type sequence or a sequence that has been modified from wild type sequence but retains promoter activity.

In some embodiments of the recombinant replicon nucleic acid of this invention, the alphavirus subgenomic promoter or 26S promoter can be a minimal or modified alphavirus subgenomic promoter (e.g., a minimal or modified alphavirus subgenomic promoter as known in the art).

In some embodiments of the recombinant replicon nucleic acid of this invention, the at least one heterologous nucleotide sequence can be an antisense sequence or it can encode a protein (which in some embodiments can be an alphavirus structural protein), or it can encodes a ribozyme.

In some embodiments of the recombinant replicon nucleic acid of this invention the nucleotide sequence of (a), the nucleotide sequence of (b) and/or the nucleotide sequence of (e) can be derived from Sindbis virus (SINV), Aura virus (AURV), Mayaro virus (MAYV), Ross River virus (RRV), Semliki Forest virus (SFV), Getah virus (GETV), or O' Nyong Nyong virus (ONNV).

Nonlimiting examples of an alphavirus of this invention include Eastern equine encephalitis virus (EEEV), Venezuelan equine encephalitis virus (VEEV), Western equine encephalitis virus (WEEV), Sindbis virus, South African Arbovirus No. 86 (S.A.AR86), Chikungunya virus, O' Nyong Nyong virus, Ross River virus, Barmah Forest virus, Everglades, Mucambo, Pixuna, Semliki Forest virus, Middelburg, Getah, Bebaru, Mayaro, Una, Okelbo, Babanki, Fort Morgan, Ndumu, Girwood S.A. virus, Sagiyama virus, Aura virus, Whataroa virus, Kyzlagach virus, Highlands J virus, Buggy Creek virus, and any other virus classified by the International Committee on Taxonomy of Viruses (ICTV) as an alphavirus, as well as subgroups thereof as are known in the art. The complete genomic sequences, as well as the sequences of the various structural and non-structural proteins, are known in the art for numerous alphaviruses and include as nonlimiting examples: Sindbis virus genomic sequence (GenBank® Accession No. J02363, NCBI Accession No. NC_001547), S.A.AR86 genomic sequence (GenBank® Accession No. U38305), VEEV genomic sequence (GenBank® Accession No. L04653, NCBI Accession No. NC_001449), Girdwood S. A genomic sequence (GenBank® Accession No. U38304), Semliki Forest virus genomic sequence GenBank® Accession No. X04129, NCBI Accession No. NC_003215), and the TR339 genomic sequence (Klimstra et al. (1988) J. Virol. 72:7357; McKnight et al. (1996) J. Virol. 70:1981). These sequences and references are incorporated by reference herein.

In further embodiments, the present invention provides a vector comprising the recombinant replicon nucleic acid of this invention, as well as a cell comprising said vector.

The alphavirus replicons of this invention can be applied as immunogens and/or for more production of a protein of interest (POI). These replicons and constructs comprising them can be used for improvement of i) DNA vaccines, if delivered in DNA form, and ii) RNA vaccines, if delivered as in vitro-synthesized RNA. The replicons of this invention can also be packaged into viral particles and delivered into cells using a natural, virion-mediated route of infection. The replicons of this invention can be applied in a protein production system for the large-scale production of heterologous proteins in eukaryotic cells (e.g., mammalian or insect cells).

Furthermore, the present invention provides a cell, which can be a host cell, a helper cell, a packaging cell or a producer cell, comprising the recombinant replicon nucleic acid of this invention.

The terms “helper,” “helper RNA” and “helper construct” are used interchangeably and refer to a nucleic acid molecule (either RNA or DNA) that encodes one or more alphavirus structural proteins. In the present invention, the helper construct generally encodes an RNA-binding competent alphavirus capsid protein. The capsid protein can comprise the amino acid sequence of what is known in the art to be the “wild type” capsid protein of a given alphavirus. Exemplary wild type amino acid sequences of various alphaviruses of this invention are provided herein. The capsid protein encoded by a helper construct of this invention can also be an alphavirus capsid protein that has the function of binding and packaging alphavirus RNA and may have other modifications that distinguish its amino acid sequence from a wild type sequence, while retaining the RNA binding and packaging function. Optionally, the helper construct of this invention does not comprise a packaging signal. Optionally, the helper construct of this invention can comprise nucleotide sequence encoding all or a portion of one or more alphavirus nonstructural proteins or the helper construct of this invention does not comprise nucleotide sequence encoding all or a portion of one or more alphavirus nonstructural proteins. Further options for the helper construct of this invention can include a helper construct comprising nucleotide sequence encoding all or a portion of one or more alphavirus structural proteins (e.g., in addition to capsid) or the helper construct does not comprise nucleotide sequence encoding one or more alphavirus structural proteins (e.g., besides capsid).

A helper nucleic acid of this invention can comprise nucleic acid sequences encoding any one or more of the alphavirus structural proteins (C, E1, E2) in any order and/or in any combination. Thus, a helper cell can comprise as many helper nucleic acids as needed in order to provide all of the alphavirus structural proteins necessary to produce alphavirus particles. A helper cell can also comprise helper nucleic acid(s) stably integrated into the genome of a helper (e.g., packaging or producer) cell. In such helper cells, the alphavirus structural proteins can be produced under the control of a promoter that can be an inducible promoter.

In some embodiments of this invention, a series of helper nucleic acids (“helper constructs” or “helper molecules”), i.e., recombinant DNA or RNA molecules that express one or more alphavirus structural proteins, are provided. In some embodiments, the E1 and E2 glycoproteins are encoded by one helper construct, and the capsid protein is encoded by another separate helper construct. In another embodiment, the E1 glycoprotein, E2 glycoprotein, and capsid protein are each encoded by separate helper constructs. In other embodiments, the capsid protein and one of the glycoproteins are encoded by one helper construct, and the other glycoprotein is encoded by a separate second helper construct. In yet further embodiments, the capsid protein and glycoprotein E1 are encoded by one helper construct and the capsid protein and glycoprotein E2 are encoded by a separate helper construct. In certain embodiments, the helper constructs of this invention do not include an alphavirus packaging signal.

Alternatively, helper nucleic acids can be constructed as DNA molecules, which can be stably integrated into the genome of a helper cell or expressed from an episome (e.g., an EBV derived episome). The DNA molecule can also be transiently expressed in a cell. The DNA molecule can be any vector known in the art, including but not limited to, a non-integrating DNA vector, such as a plasmid, or a viral vector. The DNA molecule can encode one or all of the alphavirus structural proteins, in any combination, as described herein.

The helper constructs of this invention are introduced into “helper cells,” which are used to produce the alphavirus particles of this invention. As noted above, the nucleic acids encoding alphavirus structural proteins can be present in the helper cell transiently or by stable integration into the genome of the helper cell. The nucleic acid encoding the alphavirus structural proteins that are used to produce alphavirus particles of this invention can be under the control of constitutive and/or inducible promoters. In particular embodiments, the helper cells of the invention comprise nucleic acid sequences encoding the alphavirus structural proteins in a combination and/or amount sufficient to produce an alphavirus particle of this invention when a recombinant replicon nucleic acid is introduced into the cell under conditions whereby the alphavirus structural proteins are produced and the recombinant replicon nucleic acid is packaged into alphavirus particle of this invention.

The term “alphavirus structural protein/protein(s)” refers to one or a combination of the structural proteins encoded by alphaviruses. These are produced by the wild type virus as a polyprotein and are described generally in the literature as C-E3-E2-6k-E1. E3 and 6k serve as membrane translocation/transport signals for the two glycoproteins, E2 and E1. Thus, use of the term E1 herein can refer to E1, 6k-E1, or E3-E2-6k-E1, and use of the term E2 herein can refer to E2, E3-E2, E2-6k, PE2, p62 or E3-E2-6k.

The terms “helper,” “helper RNA,” “helper molecule,” “helper nucleic acid” and “helper construct” are used interchangeably and refer to a nucleic acid molecule (either RNA or DNA) that encodes one or more alphavirus structural proteins. In the present invention, the helper construct generally encodes an RNA-binding competent alphavirus capsid protein. The capsid protein can comprise the amino acid sequence of what is known in the art to be the “wild type” capsid protein of a given alphavirus. Exemplary wild type amino acid sequences of various alphaviruses of this invention are provided herein below. The capsid protein encoded by a helper construct of this invention can also be an alphavirus capsid protein that has the function of binding and packaging alphavirus RNA and may have other modifications that distinguish its amino acid sequence from a wild type sequence, while retaining the RNA binding and packaging function. Optionally, the helper construct of this invention does not comprise a packaging signal. Optionally, the helper construct of this invention can comprise nucleotide sequence encoding all or a portion of one or more alphavirus nonstructural proteins or the helper construct of this invention does not comprise nucleotide sequence encoding all or a portion of one or more alphavirus nonstructural proteins. Further options for the helper construct of this invention can include a helper construct comprising nucleotide sequence encoding all or a portion of one or more alphavirus structural proteins (e.g., in addition to capsid) or the helper construct does not comprise nucleotide sequence encoding one or more alphavirus structural proteins (e.g., besides capsid).

The terms “helper cell” and “packaging cell” and “producer cell” are used interchangeably herein and refer to a cell in which alphavirus particles are produced. In particular embodiments, the helper cell or packaging cell or producer cell of the present invention contains a stably integrated nucleotide sequence encoding an alphavirus RNA-binding competent capsid protein. The helper cell or packaging cell or producer can be any cell that is alphavirus-permissive, i.e., that can produce alphavirus particles upon introduction of an alphavirus genome or recombinant replicon nucleic acid. Alphavirus-permissive cells of this invention include, but are not limited to, Vero, baby hamster kidney (BHK), 293, 293T/17 (ATCC accession number CRL-11268), chicken embryo fibroblast (CEF), UMNSAH/DF-1 (ATCC accession number CRL-12203) and Chinese hamster ovary (CHO) cells.

An “isolated cell” as used herein is a cell or population of cells that have been removed from the environment in which the cell occurs naturally and/or altered or modified from the state in which the cell occurs in its natural environment. An isolated cell of this invention can be a cell, for example, in a cell culture. An isolated cell of this invention can also be a cell that can be in an animal and/or introduced into an animal and wherein the cell has been altered or modified, e.g., by the introduction into the cell of an alphavirus particle of this invention.

In all of the embodiments of this invention, it is contemplated that at least one of the alphavirus structural and/or non-structural proteins encoded by the recombinant replicon nucleic acid and/or helper molecules, and/or the nontranslated regions of the recombinant replicon and/or helper nucleic acid, can contain one or more attenuating mutations in any combination, as described herein and as are well known in the literature.

The noncytopathic replication of viruses and replicons with attenuating mutation(s) in nsP2 V peptide may require further attenuation in some cell lines. In some embodiments, this can be achieved by introduction of further mutation(s) in the alphavirus nonstructural proteins. For example, mutations in the nsP3 macrodomain (e.g., N24T and N24A/D32G), which would inhibit its mono-ADP-ribosylhydrolase activity, will further attenuate a replicon without reducing its replication rate. Alternatively, additional mutations in nsP1 or nsP2, which would reduce replication rate, will also attenuate the replicon.

In some embodiments, the cell can be a helper cell, a packaging cell or a producer cell that also comprises a recombinant DNA molecule for transiently expressing alphavirus structural proteins comprising a constitutive promoter for directing the transcription of RNA from a DNA sequence operably linked to a DNA sequence comprising a complete alphavirus structural polyprotein-coding sequence.

In some embodiments, the cell can be a helper cell, a packaging cell or a producer cell that also comprises a first helper RNA encoding at least one but not all alphavirus structural proteins and a second helper RNA and optionally a third helper RNA encoding any alphavirus structural proteins not encoded by the first helper RNA or second helper RNA.

Nonlimiting examples of the alphavirus structural proteins produced in a helper cell, packaging cell or producer cell of this invention include Venezuelan equine encephalitis virus (VEE), S.A.AR 86 virus, Semliki Forest virus (SFV), Ross River virus (RRV), Sindbis virus (SINV), Aura virus (AURV), Mayaro virus (MAYV), Getah virus (GETV), Chikungunya virus (CHIKV) or O' Nyong Nyong virus (ONNV) structural proteins.

Further provided in this invention is an infectious alphavirus particle comprising the recombinant replicon nucleic acid of this invention as well as an infectious alphavirus particle produced by the above method.

The present invention further provides a method of making infectious, defective alphavirus particles, comprising: a) introducing into a cell (e.g., a helper cell, a packaging cell or a producer cell) the following: (i) a recombinant replicon nucleic acid of this invention, and (ii) one or more helper nucleic acids encoding alphavirus structural proteins, wherein the one or more helper nucleic acids produce all of the alphavirus structural proteins, and b) producing said alphavirus particles in the cell. In some embodiments, the recombinant replicon nucleic acid can comprise at least one heterologous nucleic acid encoding an alphavirus structural protein. In some embodiments the replicon nucleic acid contains a packaging signal. The methods of making alphavirus particles of this invention can further comprise the step of collecting said alphavirus particles from the cell.

Additionally, the present invention provides a composition comprising a population of infectious alphavirus replicon particles of this invention, in a pharmaceutically acceptable carrier.

Also provided herein is a composition comprising a population of attenuated alphavirus particles of this invention, wherein said particle comprises a nucleotide sequence encoding the alphavirus nsP2 protein of this invention.

In further embodiments, the present invention provides a composition comprising the mutated alphavirus nsP2 protein of this invention, the attenuated alphavirus particle of this invention, the recombinant replicon nucleic acid of this invention, the vector of this invention, the helper cell, packaging cell and/or producer cell of this invention, and/or the infectious alphavirus particle of this invention, in a pharmaceutically acceptable carrier.

Thus, the present invention provides a composition (e.g., a pharmaceutical composition) comprising a replicon nucleic acid, a nucleic acid vector, a virus particle and/or a population of alphavirus particles of this invention in a pharmaceutically acceptable carrier.

By “pharmaceutically acceptable” is meant a material that is not biologically or otherwise undesirable, i.e., the material may be administered to a subject along with the selected particles, and/or populations thereof, without causing substantial deleterious biological effects or interacting in a deleterious manner with any of the other components of the composition in which it is contained. The pharmaceutically acceptable carrier is suitable for administration or delivery to humans and other subjects of this invention. The carrier would naturally be selected to minimize any degradation of the active ingredient and to minimize any adverse side effects in the subject, as would be well known to one of skill in the art (see, e.g., Remington's Pharmaceutical Science; latest edition). Pharmaceutical formulations, such as vaccines or other immunogenic compositions of the present invention can comprise an immunogenic amount of the alphavirus particles of this invention, in combination with a pharmaceutically acceptable carrier. Exemplary pharmaceutically acceptable carriers include, but are not limited to, sterile pyrogen-free water and sterile pyrogen-free physiological saline solution.

The present invention also provides a method of delivering a nucleic acid to a cell, comprising introducing into the cell the recombinant replicon nucleic acid of this invention, the vector of this invention and/or the infectious alphavirus particle of this invention. In some embodiments, the cell can be in a subject of this invention.

In the methods of this invention, the subject can be any animal that is susceptible to infection by an alphavirus and in particular embodiments, the subject can be a human. Thus, a “subject” of this invention includes, but is not limited to, warm-blooded animals, e.g., humans, non-human primates, horses, cows, cats, dogs, pigs, rats, and mice. Administration of the various compositions of this invention (e.g., nucleic acids, particles, populations, pharmaceutical compositions) can be accomplished by any of several different routes. In specific embodiments, the compositions can be administered intramuscularly, subcutaneously, intraperitoneally, intradermally, intranasally, intracranially, sublingually, intravaginally, intrarectally, orally, or topically. The compositions herein may be administered via a skin scarification method, or transdermally via a patch or liquid. The compositions can be delivered subdermally in the form of a biodegradable material that releases the compositions over a period of time.

Additionally provided herein is a method of delivering a therapeutic heterologous protein and/or functional RNA to a subject, comprising administering to the subject the recombinant replicon nucleic acid of this invention, the vector of this invention and/or the infectious alphavirus particle of this invention, wherein the replicon nucleic acid encodes a therapeutic heterologous protein and/or functional RNA, thereby delivering a therapeutic heterologous protein and/or functional RNA to the subject.

The present invention further provides a method of producing a protein of interest (POI) (e.g., a heterologous protein) in a cell, comprising introducing into the cell the recombinant replicon nucleic acid of this invention, the vector of this invention and/or the infectious alphavirus particle of this invention, wherein the recombinant replicon nucleic acid comprises a nucleotide sequence encoding the protein of interest, under conditions whereby the recombinant replicon nucleic acid is expressed and the protein of interest is produced. In some embodiments, this method further comprises the step of harvesting the protein from a cell culture. In some embodiments, the cell can be in a subject of this invention.

In some embodiments, the term “heterologous” as used herein can include a nucleotide sequence that is not naturally occurring in the nucleic acid construct and/or delivery vector (e.g., alphavirus delivery vector) in which it is contained and can also include a nucleotide sequence that is placed into a non-naturally occurring environment and/or non-naturally occurring position relative to other nucleotide sequences (e.g., by association with a promoter or coding sequence with which it is not naturally associated).

In some embodiments, a nucleotide sequence of this invention can encode a protein, peptide and/or RNA of this invention that is heterologous (i.e., not naturally occurring, not present in a naturally occurring state and/or modified and/or duplicated (e.g., in a cell that also produces its own endogenous version of the protein, peptide and/or RNA)) to the cell into which it is introduced. The nucleotide sequence can also be heterologous to the vector (e.g., an alphavirus vector) into which it is placed.

Alternatively, the protein, peptide or RNA (e.g., a heterologous protein, peptide or functional RNA of interest) encoded by the heterologous nucleotide sequence of interest can comprise, consist essentially of, or consist of a nucleotide sequence that may otherwise be endogenous to the cell (i.e., one that occurs naturally in the cell) but is introduced into and/or is present in the cell as an isolated heterologous nucleic acid.

Furthermore, the present invention provides a method of inducing and/or enhancing an immune response in a subject, comprising administering to the subject an effective amount of the attenuated alphavirus particle of this invention, the recombinant replicon nucleic acid of this invention, the vector of this invention, the cell of this invention, and/or the infectious alphavirus particle of this invention, thereby inducing and/or enhancing an immune response in the subject as compared with a control subject. In some embodiments, a control subject can be a subject to whom the attenuated alphavirus particle of this invention, the recombinant replicon nucleic acid of this invention, the vector of this invention, the cell of this invention, and/or the infectious alphavirus particle of this invention has not been administered.

A method is also provided herein of treating and/or preventing an alphavirus infection and/or treating the effects of an alphavirus infection in a subject, comprising administering to the subject an immunogenic amount of the attenuated alphavirus particle of this invention, the recombinant replicon nucleic acid of this invention, the vector of this invention, the cell of this invention, and/or the infectious alphavirus particle of this invention, thereby treating and/or preventing an alphavirus infection in the subject and/or treating the effects of an alphavirus infection in the subject.

“Treat” or “treating” or “treatment” refers to any type of action that imparts a modulating effect, which, for example, can be a beneficial effect, to a subject afflicted with a disorder, disease or illness, including improvement in the condition of the subject (e.g., in one or more symptoms), delay or reduction in the progression of the condition, delay of the onset of the disorder, disease or illness, and/or change in any of the clinical parameters of a disorder, disease or illness, etc., as would be well known in the art.

The terms “preventing” or “prevent” as used herein refers to the prophylactic administration of the alphavirus PIV particles of this invention to a subject to protect the subject from becoming infected by the alphavirus and/or to reduce the severity of an alphavirus infection in a subject who becomes infected. Such as subject can be a healthy subject for whom prevention of infection by an alphavirus is desirable. The subject can also be at increased risk of becoming infected by an alphavirus and therefore desires and/or is in need of the methods of preventing alphavirus infection provided herein.

An “immunogenic amount” is an amount of the alphavirus particle in the populations of this invention that is sufficient to elicit, induce and/or enhance an immune response in a subject to which the population of particles is administered or delivered. An amount of from about 10⁴ to about 10⁹, especially 10⁶ to 10⁸, infectious units, or “IU,” as determined by assays well known in the art, per dose is considered suitable, depending upon the age and species of the subject being treated. Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time.

As used herein, “effective amount” refers to an amount of a population or composition or formulation of this invention that is sufficient to produce a desired effect, which can be a therapeutic effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation. (See, for example, Remington, The Science And Practice of Pharmacy (20th ed. 2000)).

Alternatively, pharmaceutical formulations of the present invention may be suitable for administration to the mucous membranes of a subject (e.g., via intranasal administration, buccal administration and/or inhalation). The formulations may be conveniently prepared in unit dosage form and may be prepared by any of the methods well known in the art.

Immunogenic compositions comprising a population of the particles of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable.

The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars.

In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

Furthermore, any of the compositions of this invention can comprise a pharmaceutically acceptable carrier and a suitable adjuvant. As used herein, “suitable adjuvant” describes an adjuvant capable of being combined with the peptide or polypeptide of this invention to further enhance an immune response without deleterious effect on the subject or the cell of the subject. A suitable adjuvant can be, but is not limited to, MONTANIDE ISA51 (Seppic, Inc., Fairfield, N.J.), SYNTEX adjuvant formulation 1 (SAF-1), composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Other suitable adjuvants are well known in the art and include QS-21, Freund's adjuvant (complete and incomplete), aluminum salts (alum), aluminum phosphate, aluminum hydroxide, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-iso glutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Adjuvants can be combined, either with the compositions of this invention or with other vaccine compositions that can be used in combination with the compositions of this invention. Examples of adjuvants can also include, but are not limited to, oil-in-water emulsion formulations, immunostimulating agents, such as bacterial cell wall components or synthetic molecules, or oligonucleotides (e.g. CpGs) and nucleic acid polymers (both double stranded and single stranded RNA and DNA), which can incorporate alternative backbone moieties, e.g., polyvinyl polymers.

The compositions of the present invention can also include other medicinal agents, pharmaceutical agents, carriers, diluents, immunostimulatory cytokines, etc. Actual methods of preparing such dosage forms are known, or will be apparent, to those skilled in this art. Preferred dosages for alphavirus replicon particles, as contemplated by this invention, can range from 10³ to 10¹⁰ particles per dose. For humans, 10⁶, 10⁷ or 10⁸ particles are preferred doses. A dosage regimen can be one or more doses hourly, daily, weekly, monthly, yearly, etc. as deemed necessary to achieve the desired prophylactic and/or therapeutic effect to be achieved by administration of a composition of this invention to a subject. The efficacy of a particular dosage can be determined according to methods well known in the art.

Additional examples of adjuvants can include, but are not limited to, immunostimulating agents, such as bacterial cell wall components or synthetic molecules, or oligonucleotides (e.g., CpGs) and nucleic acid polymers (both double stranded and single stranded RNA and DNA), which can incorporate alternative backbone moieties, e.g., polyvinyl polymers.

The effectiveness of an adjuvant may be determined by measuring the amount of antibodies or cytotoxic T-cells directed against the immunogenic product of the alphavirus PIV particles resulting from administration of the particle-containing composition in a vaccine formulation that also comprises an adjuvant or combination of adjuvants. Such additional formulations and modes of administration as are known in the art may also be used.

Adjuvants can be combined, either with the compositions of this invention or with other vaccine formulations that can be used in combination with the compositions of this invention.

The compositions of the present invention can also include other medicinal agents, pharmaceutical agents, carriers, and diluents.

The compositions of this invention can be optimized and combined with other vaccination regimens to provide the broadest (i.e., covering all aspects of the immune response, including those features described hereinabove) cellular and humoral responses possible. In certain embodiments, this can include the use of heterologous prime-boost strategies, in which the compositions of this invention are used in combination with a composition comprising one or more of the following: immunogens derived from a pathogen or tumor, recombinant immunogens, naked nucleic acids, nucleic acids formulated with lipid-containing moieties, non-alphavirus vectors (including but not limited to pox vectors, adenoviral vectors, adeno-associated viral vectors, herpes virus vectors, vesicular stomatitis virus vectors, paramyxoviral vectors, parvovirus vectors, papovavirus vectors, retroviral vectors, lentivirus vectors), and other alphavirus vectors.

The immunogenic (or otherwise biologically active) alphavirus particle-containing populations and compositions of this invention are administered in a manner compatible with the dosage formulation, and in such amount as will be prophylactically and/or therapeutically effective. The quantity to be administered, which can generally be in the range of about 10⁴ to about 10¹⁰ infectious units in a dose (e.g., about 10⁴, about 10⁵, about 10⁶, about 10⁷, about 10⁸, about 10⁹, or about 10¹⁰), depends on the subject to be treated, the route by which the particles are administered or delivered, the immunogenicity of the expression product, the types of effector immune responses desired, and the degree of protection desired. In some embodiments, doses of about 10⁶, about 10⁷, and about 10⁸ infectious units may be particularly effective in human subjects. Effective amounts of the active ingredient required to be administered or delivered may depend on the judgment of the physician, veterinarian or other health practitioner and may be specific for a given subject, but such a determination is within the skill of such a practitioner.

The compositions and formulations of this invention may be given in a single dose or multiple dose schedule. A multiple dose schedule is one in which a primary course of administration may include 1 to 10 or more separate doses, followed by other doses administered at subsequent time intervals as required to maintain and or reinforce the desired effect (e.g., an immune response), e.g., weekly or at 1 to 4 months for a second dose, and if needed, a subsequent dose(s) after several months (e.g., 4 or 6 months)/years.

Efficacy of the treatment methods of this invention can be determined according to well-known protocols for determining the outcome of a treatment of a disease or infection of this invention. Determinants of efficacy of treatment, include, but are not limited to, overall survival, disease-free survival, improvement in symptoms, time to progression and/or quality of life, etc., as are well known in the art.

Also, the composition of this invention may be used to infect or be transfected into dendritic cells, which are isolated or grown from a subject's cells, according to methods well known in the art, or onto bulk peripheral blood mononuclear cells (PBMC) or various cell subfractions thereof from a subject.

If ex vivo methods are employed, cells or tissues can be removed and maintained outside the body according to standard protocols well known in the art while the compositions of this invention are introduced into the cells or tissues.

As used herein, “eliciting an immune response” and “immunizing a subject” includes the development, in a subject, of a humoral and/or a cellular immune response to a protein and/or polypeptide of this invention (e.g., an immunogen, an antigen, an immunogenic peptide, and/or one or more epitopes). A “humoral” immune response, as this term is well known in the art, refers to an immune response comprising antibodies, while a “cellular” immune response, as this term is well known in the art, refers to an immune response comprising T-lymphocytes and other white blood cells, especially the immunogen-specific response by HLA-restricted cytolytic T-cells, i.e., “CTLs.” A cellular immune response occurs when the processed immunogens, i.e., peptide fragments, are displayed in conjunction with the major histocompatibility complex (MHC) HLA proteins, which are of two general types, class I and class II. Class I HLA-restricted CTLs generally bind 9-mer peptides and present those peptides on the cell surface. These peptide fragments in the context of the HLA Class I molecule are recognized by specific T-Cell Receptor (TCR) proteins on T-lymphocytes, resulting in the activation of the T-cell. The activation can result in a number of functional outcomes including, but not limited to expansion of the specific T-cell subset resulting in destruction of the cell bearing the HLA-peptide complex directly through cytotoxic or apoptotic events or the activation of non-destructive mechanisms, e.g., the production of interferon/cytokines. Presentation of immunogens via Class I MHC proteins typically stimulates a CD8+ CTL response.

Another aspect of the cellular immune response involves the HLA Class II-restricted T-cell responses, involving the activation of helper T-cells, which stimulate and focus the activity of nonspecific effector cells against cells displaying the peptide fragments in association with the MHC molecules on their surface. At least two types of helper cells are recognized: T-helper 1 cells (Th1), which secrete the cytokines interleukin 2 (IL-2) and interferon-gamma and T-helper 2 cells (Th2), which secrete the cytokines interleukin 4 (IL-4), interleukin 5 (IL-5), interleukin 6 (IL-6) and interleukin 10 (IL-10). Presentation of immunogens via Class II MHC proteins typically elicits a CD4+ CTL response as well as stimulation of B lymphocytes, which leads to an antibody response.

An “immunogenic polypeptide,” “immunogenic peptide,” or “immunogen” as used herein includes any peptide, protein or polypeptide that elicits an immune response in a subject and in certain embodiments, the immunogenic polypeptide is suitable for providing some degree of protection to a subject against a disease. These terms can be used interchangeably with the term “antigen.”

In certain embodiments, the immunogen of this invention can comprise, consist essentially of, or consist of one or more “epitopes.” An “epitope” is a set of amino acid residues that is involved in recognition by a particular immunoglobulin. In the context of T cells, an epitope is defined as the set of amino acid residues necessary for recognition by T cell receptor proteins and/or MHC receptors. In an immune system setting, in vivo or in vitro, an epitope refers to the collective features of a molecule, such as primary, secondary and/or tertiary peptide structure, and/or charge, that together form a site recognized by an immunoglobulin, T cell receptor and/or HLA molecule. In the case of a B-cell (antibody) epitope, it is typically a minimum of 3-4 amino acids, preferably at least 5, ranging up to approximately 50 amino acids. Preferably, the humoral response-inducing epitopes are between 5 and 30 amino acids, usually between 12 and 25 amino acids, and most commonly between 15 and 20 amino acids. In the case of a T-cell epitope, an epitope includes at least about 7-9 amino acids, and for a helper T-cell epitope, at least about 12-20 amino acids. Typically, such a T-cell epitope will include between about 7 and 15 amino acids, e.g., 7, 8, 9, 10, 11, 12, 13, 14 or 15 amino acids.

The present invention can be employed to express a nucleic acid encoding an immunogenic polypeptide in a subject (e.g., for vaccination) or for immunotherapy (e.g., to treat a subject with cancer or tumors). Thus, in the case of vaccines, the present invention thereby provides methods of eliciting or inducing or enhancing an immune response in a subject, comprising administering to the subject an immunogenic amount of a nucleic acid, particle, population and/or composition of this invention.

It is also contemplated that the nucleic acids, particles, populations and pharmaceutical compositions of this invention can be employed in methods of delivering a nucleic acid of interest (NOI) to a cell, which can be a cell in a subject. Thus, the present invention provides a method of delivering a heterologous nucleic acid to a cell comprising introducing into a cell an effective amount of a nucleic acid, particle, population and/or composition of this invention. Also provided is a method of delivering a heterologous nucleic acid to a cell in a subject, comprising delivering to the subject an effective amount of a nucleic acid, particle, population and/or composition of this invention. Such methods can be employed to impart a therapeutic effect on a cell and/or a subject of this invention, according to well-known protocols for gene therapy.

In some embodiments, the heterologous nucleic acid of this invention can encode a protein or peptide and in some embodiments the heterologous nucleic acid of this invention can encode a functional RNA, as is well known in the art.

The heterologous nucleic acid of this invention can encode a protein or peptide, which can be, but is not limited to, an antigen, an immunogen or immunogenic polypeptide or peptide, a fusion protein, a fusion peptide, a cancer antigen, etc. Examples of proteins and/or peptides encoded by the heterologous nucleic acid of this invention include, but are not limited to, immunogenic polypeptides and peptides suitable for protecting a subject against a disease, including but not limited to microbial, bacterial, protozoal, parasitic, and viral diseases.

In some embodiments, for example, the protein or peptide encoded by the heterologous nucleic acid can be an orthomyxovirus immunogen (e.g., an influenza virus protein or peptide such as the influenza virus hemagglutinin (HA) surface protein or the influenza virus nucleoprotein, or an equine influenza virus protein or peptide), or a parainfluenza virus immunogen, or a metapneumovirus immunogen, or a respiratory syncytial virus immunogen, or a rhinovirus immunogen, a lentivirus immunogen (e.g., an equine infectious anemia virus protein or peptide, a Simian Immunodeficiency Virus (SIV) protein or peptide, or a Human Immunodeficiency Virus (HIV) protein or peptide, such as the HIV or SIV envelope GP160 protein, the HIV or SIV matrix/capsid proteins, and the HIV or SIV gag, pol and env gene products). The protein or peptide can also be an arenavirus immunogen (e.g., Lassa fever virus protein or peptide, such as the Lassa fever virus nucleocapsid protein and the Lassa fever envelope glycoprotein), a picornavirus immunogen (e.g., a Foot and Mouth Disease virus protein or peptide), a poxvirus immunogen (e.g., a vaccinia protein or peptide, such as the vaccinia L1 or L8 protein), an orbivirus immunogen (e.g., an African horse sickness virus protein or peptide), a flavivirus immunogen (e.g., a yellow fever virus protein or peptide, a West Nile virus protein or peptide, or a Japanese encephalitis virus protein or peptide), a filovirus immunogen (e.g., an Ebola virus protein or peptide, or a Marburg virus protein or peptide, such as NP and GP proteins), a bunyavirus immunogen (e.g., RVFV, CCHF, and SFS proteins or peptides), or a coronavirus immunogen (e.g., an infectious human coronavirus protein or peptide, such as the human coronavirus envelope glycoprotein, or a porcine transmissible gastroenteritis virus protein or peptide, or an avian infectious bronchitis virus protein or peptide). The protein or polypeptide encoded by the heterologous nucleic acid of this invention can further be a polio antigen, herpes antigen (e.g., CMV, EBV, HSV antigens) mumps antigen, measles antigen, rubella antigen, varicella antigen, botulinum toxin, diphtheria toxin or other diphtheria antigen, pertussis antigen, hepatitis (e.g., Hepatitis A, Hepatitis B, Hepatitis C, Hepatitis D, or Hepatitis E) antigen, or any other vaccine antigen known in the art.

The compositions of this invention can be used prophylactically to prevent disease or therapeutically to treat disease. Diseases that can be treated include infectious disease caused by viruses, bacteria, fungi or parasites, and cancer. Chronic diseases involving the expression of aberrant or abnormal proteins or the over-expression of normal proteins, can also be treated, e.g., Alzheimer's disease, multiple sclerosis, stroke, etc.

The replicons, particles and/or compositions of this invention can be optimized and combined with other vaccination regimens to provide the broadest (i.e., all aspects of the immune response, including those features described herein) cellular and humoral responses possible. In certain embodiments, this can include the use of heterologous prime-boost strategies, in which the compositions of this invention are used in combination with a composition comprising one or more of the following: immunogens derived from a pathogen or tumor, recombinant immunogens, naked nucleic acids, nucleic acids formulated with lipid-containing moieties, non-alphavirus vectors (including but not limited to pox vectors, adenoviral vectors, herpes vectors, vesicular stomatitis virus vectors, paramyxoviral vectors, parvovirus vectors, papovavirus vectors, retroviral vectors), and other alphavirus vectors. The viral vectors can be virus-like particles or nucleic acids. The alphavirus vectors can be replicon-containing particles, DNA-based replicon-containing vectors (sometimes referred to as an “ELVIS” system, see, for example, U.S. Pat. No. 5,814,482) or naked RNA vectors.

The compositions of the present invention can also be employed to produce an immune response against chronic or latent infectious agents, which typically persist because they fail to elicit a strong immune response in the subject. Illustrative latent or chronic infectious agents include, but are not limited to, hepatitis B, hepatitis C, Epstein-Barr Virus, herpes viruses, human immunodeficiency virus, and human papilloma viruses. Alphavirus vectors encoding peptides and/or proteins from these infectious agents can be administered to a cell or a subject according to the methods described herein.

Alternatively, the immunogenic protein or peptide can be any tumor or cancer cell antigen. Preferably, the tumor or cancer antigen is expressed on the surface of the cancer cell. Exemplary cancer antigens for specific breast cancers are the HER2 and BRCA1 antigens. Other illustrative cancer and tumor cell antigens are described in S. A. Rosenberg, (1999) Immunity 10:281) and include, but are not limited to, MART-1/MelanA, gp100, tyrosinase, TRP-1, TRP-2, MAGE-1, MAGE-3, GAGE-1/2, BAGE, RAGE, NY-ESO-1, CDK-4, β-catenin, MUM-1, Caspase-8, KIAA0205, HPVE&, SART-1, PRAME, p15 and p53 antigens, Wilms' tumor antigen, tyrosinase, carcinoembryonic antigen (CEA), prostate specific antigen (PSA), prostate-specific membrane antigen (PSMA), prostate stem cell antigen (PSCA), human aspartyl (asparaginyl) β-hydroxylase (HAAH), and EphA2 (an epithelial cell tyrosine kinase, see International Patent Publication No. WO 01/12172).

The immunogenic polypeptide or peptide of this invention can also be a “universal” or “artificial” cancer or tumor cell antigen as described in international patent publication WO 99/51263, which is incorporated herein by reference in its entirety for the teachings of such antigens.

Further provided herein is a method of screening a test agent and/or compound for anti-alphavirus activity, comprising: a) generating a cell line in which the recombinant replicon nucleic acid of this invention, encoding a marker protein such as green fluorescent protein (GFP) or luciferase, is persistently replicated; b) introducing into cells of this cell line a test agent and/or compound; and c) observing the effect of the presence of the test agent and/or compound on expression of the marker protein in the cell to evaluate the effect of the test agent and/or compound on the ability of the recombinant replicon nucleic acid to replicate, thereby identifying a test agent or compound that inhibits (e.g., as evidenced by decreased marker signal) or enhances (e.g., as evidenced by increased marker signal) recombinant replicon nucleic acid replication.

The present subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### EXAMPLES

The following EXAMPLES provide illustrative embodiments. Certain aspects of the following EXAMPLES are disclosed in terms of techniques and procedures found or contemplated by the present inventors to work well in the practice of the embodiments. In light of the present disclosure and the general level of skill in the art, those of skill will appreciate that the following EXAMPLES are intended to be exemplary only and that numerous changes, modifications, and alterations can be employed without departing from the scope of the presently claimed subject matter.

### Example 1: Inhibition of Cellular Transcription and Translation are Redundant Determinants of Cell Death During Sindbis Virus Infection

Alphaviruses are a group of small enveloped viruses with an RNA genome of positive polarity. In nature, they are transmitted by mosquito vectors between vertebrate hosts. In mosquitoes, they cause persistent, life-long infection that does not have detectable negative effect on insect biology. In vertebrates, alphaviruses cause diseases of different severity, characterized by rapid development of high titer viremia that is required for infecting new mosquitoes during the blood meal. Alphavirus replication in vitro mirrors the infection in vivo. These viruses develop persistent replication in cultured mosquito cells and a highly cytopathic infection in cells of vertebrate origin Within 3-4 hours post infection, the latter cells already release infectious virus particles, which perform the next round of infection. This rapid development of spreading infection is mediated by multiple mechanisms. First, the alphavirus replication machinery is highly efficient and within 4-6 h post infection (PI) the numbers of virus-specific RNAs can approach 10⁵ molecules per cell, subsequently, within the next few hours each infected cell releases 10³-10⁴ virions. Second, to promote the spread of infection, alphaviruses downregulate certain cell signaling pathways and primarily the release of type I interferon (IFN) that can activate an antiviral state in yet uninfected cells and thus prevent dissemination of infection.

Based on their geographical circulation areas, alphaviruses are divided into two groups: the Old World (OW) and the New World (NW) alphaviruses. The NW alphaviruses include Venezuelan, eastern and western equine encephalitis viruses (VEEV, EEEV and WEEV, respectively). They cause sporadic outbreaks in South, Central and North Americas and develop meningoencephalitis with high mortality rates in humans. The OW alphaviruses are more broadly distributed, but usually cause a self-limited febrile illness. However, chikungunya (CHIKV), O' Nyong Nyong (ONNV) and Ross River (RRV) viruses are capable of producing excruciating joint pain and severe, persistent polyarthritis. In recent years, CHIKV has significantly broadened its circulation area, causing an increase in the numbers of human infections in both hemispheres and also in the US. The Old World alphaviruses such as Sindbis (SINV), Semliki Forest (SFV) and chikungunya viruses exhibit a number of common characteristics. Therefore, for decades SINV and SFV served as good models for studying alphavirus-host interactions and molecular mechanism of virus replication.

The alphavirus genomic RNA (G RNA) is approximately 11.5 kb in length. G RNA mimics the structure of cellular mRNAs, in that it contains both a 5′ methylguanylate cap (cap0) and a 3′ poly(A) tail. The 5′ two-thirds of the genome is translated into 4 nonstructural proteins (nsPs) that comprise the viral components of the replication complex (vRC). The latter complex mediates replication of G RNA and transcription of the subgenomic RNA (SG RNA). The SG RNA is translated into the viral structural proteins. The structural protein-encoding genes can be deleted or replaced by heterologous genes, and upon delivery into the cells, such modified genomes (replicons) are capable of replication and expression of the heterologous proteins. Therefore, replicons are widely used in research for expression of heterologous genes and as a vaccine platform. They also represent an important tool for dissecting different aspects of virus-host interactions in the absence of high level expression of viral structural proteins.

SINV, CHIKV and SFV replicons, which lack viral structural genes, remain highly cytopathic. However, several mutations identified in the nsP2-coding sequence were capable of making the OW alphavirus replicons and some of the corresponding viruses very inefficient inducers of cytopathic effect (CPE). Mutation of P726 in the SINV nsP2 protein not only strongly reduces cytopathogenicity of the virus and corresponding replicon, but also inhibits virus replication. We have previously shown that OW alphavirus nsP2 proteins are responsible for inhibition of host transcription. After migration into the nucleus, wt nsP2 induces rapid and complete degradation of the catalytic subunit of cellular RNA polymerase II, RPB1. The transcription inhibition induces cell death, prevents type I IFN release, despite efficient detection of the OW alphavirus replication by cellular pattern recognition receptors RIG-I and MDA5. Within 2-4 h post infection, cells also become unable to activate interferon stimulated genes (ISGs) and respond to IFN-β treatment. The P726 mutation in SINV nsP2 completely abrogated the ability of nsP2 to induce RPB1 degradation. Mutation of a corresponding proline in SFV also strongly reduced virus cytopathogenicity. However, a similar mutation in CHIKV appears to be strain specific and additional mutations that lead to strong reduction in virus replication are needed to make CHIK replicon noncytopathic. Unfortunately, the effect of this mutation in SFV and CHIKV on RPB1 degradation has not been evaluated.

An important characteristic of previously selected noncytopathic SINV replicons was their extremely low level of replication. Thus, the mutated SINV nsP2 not only lost its nuclear functions, but also became an inefficient RC component. Noncytopathic SFV and CHIKV replicons also demonstrated a dramatic decrease in RNA replication, suggesting this effect as a common mechanism of attenuation. Thus, a lower RNA replication level was likely the second important contributor to the development of a less cytopathic phenotype.

Thus, in prior studies, we dissected the critical role of the OW alphavirus nsP2 transcription inhibition in CPE development, but other mechanisms contributing to the development of this phenomenon likely remained obscure because of the nonspecific effect of nsP2 mutations on replicon and virus replication.

In this study, we further dissected the mechanisms involved in CPE development during SINV replication in vertebrate cells. The newly developed SINV mutants remained capable of efficient replication, but demonstrated a variety of new characteristics in virus-cell interactions. Our new data demonstrate that defined mutations in a small surface-exposed loop of the protease domain of SINV nsP2 have a deleterious effect on its ability to induce RPB1 degradation and to inhibit host transcription. But these nsP2-specific mutations did not make SINV noncytopathic and allowed us to further dissect another component of SINV-specific CPE development. The SINV nsP3-specific macrodomain was found to be involved in regulation of translation in SINV-infected cells. The identified adaptive mutations in this domain did not affect the rates of SINV RNA and virus replication and were not sufficient to prevent virus induced CPE. However, combining of defined nsP2- and nsP3-specific mutations in the SINV genome abolished the ability of the virus to induce CPE and inhibit cell signaling.

Selection of SINV nP2 mutants incapable of inducing cytopathic effect. Previously, the main approach for selection of noncytopathic alphavirus replicons was based on using the dominant selectable markers, such as genes of puromycin acetyl transferase (Pac) or aminoglycoside 3′-phosphotransferase, APT 3′ II (Neo), the products of which make cells resistant to puromycin and G418, respectively. These genes were usually cloned into alphavirus genomes to replace those encoding structural proteins that are generally dispensable for G RNA replication. Upon delivery into the cells, these modified alphavirus G RNAs start replication and expression of the cloned heterologous genes. After following application of the drugs, some of the cells die because they do not contain a replicon and remain sensitive to selection (Pur^{S} or Neo^{S}), but most of them die because of the cytopathic nature of the OW alphavirus replicons despite being resistant to the drugs (Pur^{R} or Neo^{R}). However, a very few cells survive the selection and develop Pur^{R} or Neo^{R} foci. The corresponding foci-specific replicons contain adaptive mutations, which make them noncytopathic and still capable of expressing the selectable marker genes. Identification of these adaptive mutations provides critical information about the mechanism underlying development of virus-specific CPE.

This approach has been successfully applied for SINV-based replicons, but only mutation at P726 of nsP2 has been unambiguously shown to be responsible for the noncytopathic phenotype. Mutations at this position abrogated the nsP2-mediated degradation of RPB1, the catalytic subunit of cellular DNA-dependent RNA polymerase II. However, the same mutations of P726 in the context of SINV or its replicons also had a very strong negative effect on RNA replication rates. Attempts to select noncytopathic SFV- or CHIKV-specific replicons only yielded replicons with severely compromised replication rates, and none of the identified mutations have been evaluated for effect on RPB1 degradation. This negative effect on RNA replication rates strongly complicated the studies aiming to reveal a mechanism of CPE development and prevented dissection of its other components besides inhibition of transcription by the nuclear fraction of nsP2.

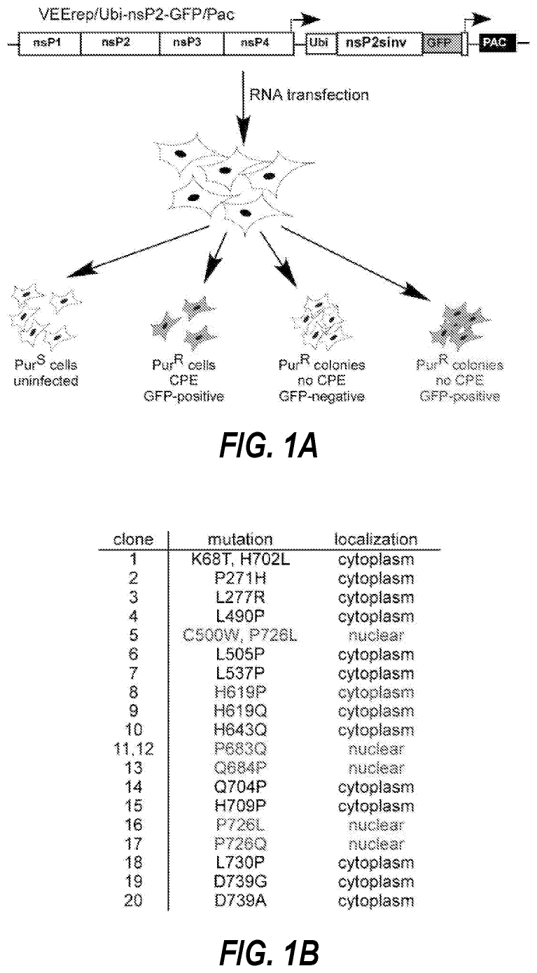

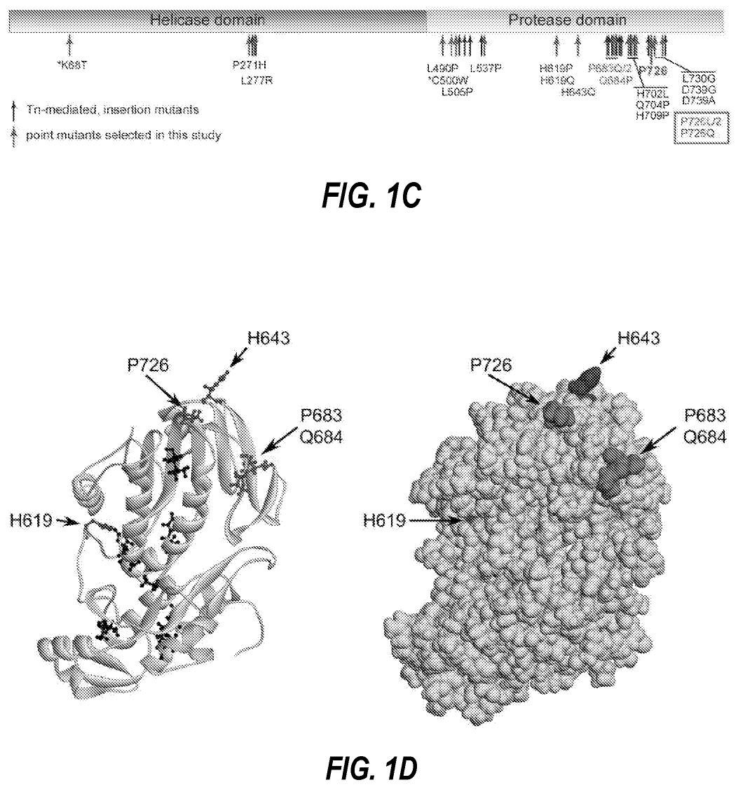

In this study, we applied a new experimental system that was aimed at selection of spontaneously developing SINV nsP2 point mutants that were no longer capable of RPB1 degradation, but could efficiently function as RC components in RNA replication. For selection of such mutants, we used a VEEV replicon encoding SINV nsP2-green fluorescent protein (GFP) fusion under control of the first subgenomic promoter and Pac gene under control of another one (FIG. 1A). VEEV replicons are not cytopathic per se, because their nsPs, including nsP2, have no nuclear functions. However, we have previously shown that expression of SINV, CHIKV and SFV naP2 with a natural first amino acid, achieved by using a Ubi-nsP2 fusion cassette, efficiently induced RPB1 degradation and rapid cell death. Fusion of the carboxy terminus of nsP2 with GFP does not affect nuclear inhibitory functions of the latter protein, but allows for monitoring of its intracellular distribution. BHK-21 cells were electroporated with the in vitro synthesized replicon RNA, and at 24 h post electroporation, puromycin selection was applied. Within a few days, we detected formation of ˜100 foci of Pur^{R} cells. A large fraction of them did not demonstrate GFP expression, suggesting alterations in the open-reading frame. Twenty one cell clones with detectable GFP expression, which were generated in two independent electroporations, were selected for further analysis. In six of them, nsP2-GFP accumulated in the nuclei with some fraction present in the cytoplasm. All others exhibited predominantly cytoplasmic distribution of nsP2-GFP. SINV nsP2-coding regions of all of the selected, noncytopathic replicons were sequenced and identified mutations are presented in FIG. 1B and FIG. 1C. Distribution of the mutations was compared to that found in the previous experiments, which were based on transposon (Tn)-based mutagenesis that randomly introduces 5 aa-long sequences into SINV nsP2 (FIG. 1C). Despite providing very important information at the time of that study, the effects of the insertions were difficult to interpret. Most of them affected both nsP2 nuclear function and virus growth, and the attempts to select SINV variants with wt replication rates, but having no nuclear functions, were unsuccessful.

In this study, three out of six replicons expressing primarily a nuclear form of nsP2-GFP had different mutations of the same P726 in SINV nsP2, additionally supporting a critical role of this amino acid in nsP2 nuclear function presented in prior studies. Since we have previously shown that any mutation of P726 strongly affects SINV replication rates, these new mutations were excluded from further experiments. The other three replicons acquired point mutations at different sites (P683Q and Q684P). Importantly, these two mutation sites overlapped with the positions of the peptide insertions identified in our random insertion mutagenesis screen. Both mutated amino acids were located on the surface of nsP2 protease domain and were in close proximity to previously investigated P726 (FIG. 1D).

The majority of other mutations in SINV nsP2 demonstrating cytoplasmic distribution were found in its C-terminal protease domain. None of them affected potential nuclear localization signals, despite making nsP2-GFP incapable of translocation to the nucleus. According to a SINV protease domain 3D model, which was based on the published crystal structure, only three mutated amino acids (H619P, H619Q and H643Q) were predicted to be solvent exposed on the surface of nsP2 (FIG. 1D). All other mutated amino acids were buried in the hydrophobic cores and thus, likely impaired the overall conformation of the protease domain. Based on our previous experience, these mutations are likely to have deleterious effects on nsP2 function in RNA replication, and their effects were not investigated further.

Three other identified mutations (K68T, P271H and L277R) were in the helicase domain of SINV nsP2. They also caused SINV nsP2-GFP to be exclusively cytoplasmic. Interestingly, the P271H and L277R mutations overlapped with several mutations identified previously by random insertion mutagenesis (FIG. 1C). These mutations were expected to affect nsP2 helicase activity that is essential not only for nsP2-mediated RPB1 degradation, but also for RNA replication. Thus, they likely would also lead to deleterious effects on SINV replication and were excluded from further study.

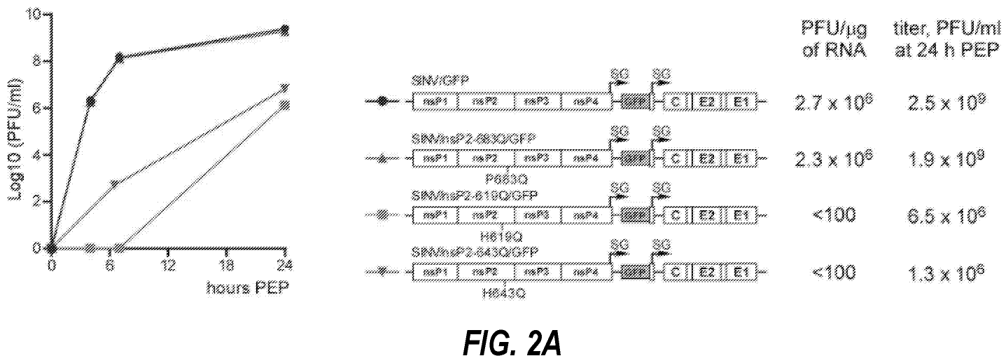

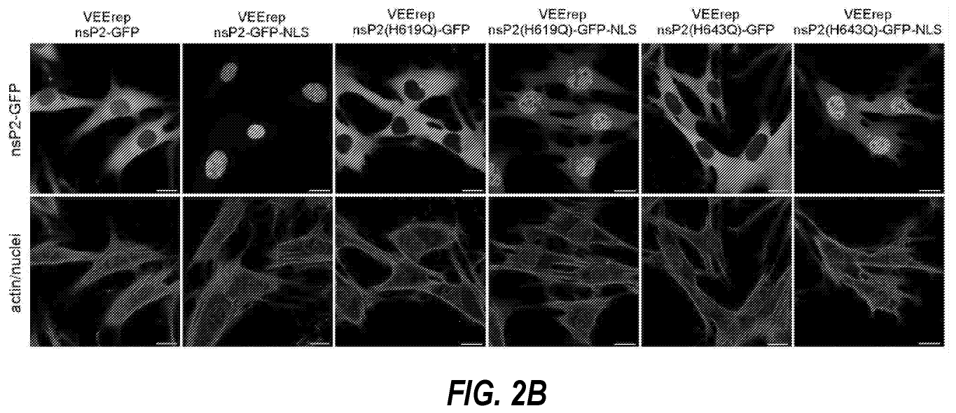

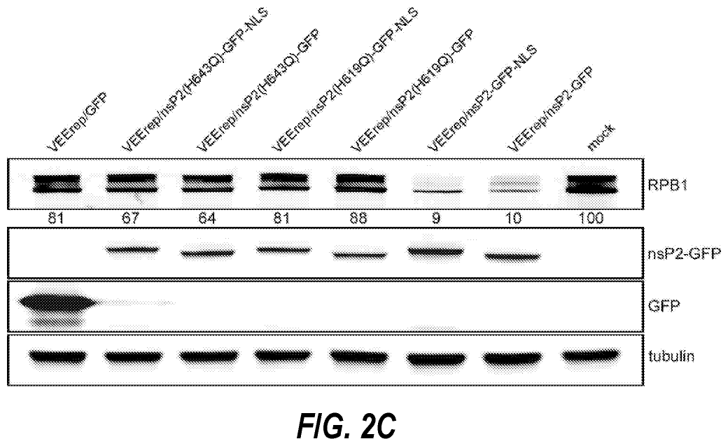

Characterization of viruses with selected mutations. In this study, we focused on three selected mutations, namely H619Q, H643Q and P683Q that were located in the protease domain and were predicted to be at least partially solvent exposed (FIG. 1D). The identified mutations were introduced into the genome of wt SINV/GFP, and the corresponding variants, SINV/nsP2-619Q/GFP, SINV/nsP2-643Q/GFP and SINV/nsP2-683Q/GFP, and control wt SINV/GFP were rescued by electroporation of the in vitro synthesize RNA into BHK-21 cells. Media were collected at different times post-electroporation to evaluate infectious titers. SINV/nsP2-683Q/GFP demonstrated replication rates that were indistinguishable from those of wt SINV/GFP (FIG. 2A). In the infectious center assay (ICA), the in vitro-synthesized RNAs of SINV/GFP and SINV/nsP2-683Q/GFP exhibited the same infectivity, suggesting that the latter designed mutant did not require additional mutations for its viability. Two other mutants, SINV/nsP2-619Q/GFP and SINV/nsP2-643Q/GFP, were not viable. Very few GFP-positive cells, which ultimately developed plaques, were detected in the ICA, and sequencing of viral nsP2 genes from the randomly selected plaques identified them as true revertants.

The H619Q and H643Q mutations were additionally characterized in terms of their effect on SINV nsP2's ability to cause RPB1 degradation. Based on the original screen, SINV nsP2-GFP containing either of these mutations and expressed by VEEV replicons was distributed mostly in the cytoplasm (FIG. 1B). Therefore, to additionally understand effects of the mutations on nuclear functions of SINV nsP2, the mutated nsP2-GFP cassettes expressed by VEEV replicons were designed to either contain or have no additional nuclear localization signal (NLS) at the C-terminus of GFP. The final replicon constructs were packaged into VEEV structural proteins and then used to infect naive cells. Addition of NLS caused an accumulation of almost all of the nsP2-GFP in nuclei (FIG. 2B). However, in contrast to wt nsP2-GFP or its fusion with NLS, none of the mutated nsP2 fusions caused RPB1 degradation (FIG. 2C). Thus, H619Q and H643Q mutations abrogated SINV nsP2 nuclear functions even if it was efficiently transported into the nuclei.

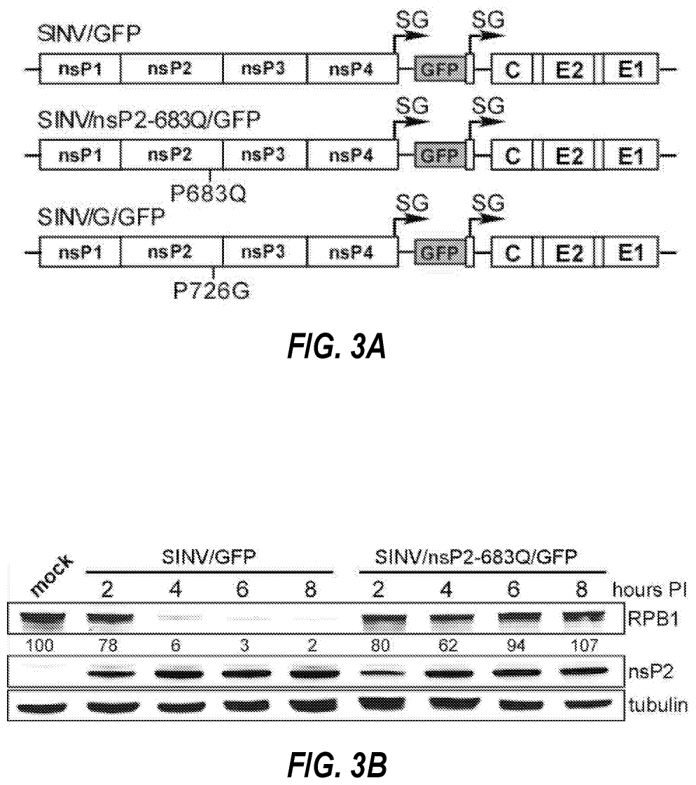

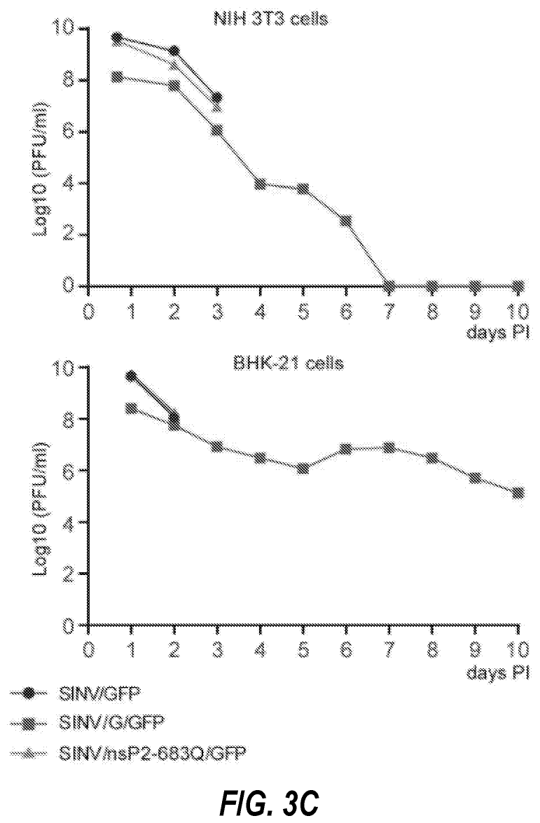

Mutations of P683 prevent nsP2-mediated RPB1 degradation, but not CPE development. The described above P683Q mutation made SINV nsP2-GFP expressed from VEEV replicon, noncytopathic and did not demonstrate a detectable negative effect on replication of SINV at least in BHK-21 cells. This was the first indication that the latter mutation could affect the transcription inhibition component of SINV-specific CPE, while having no effect on other virus-specific mechanisms of CPE induction. To experimentally confirm this hypothesis, we compared the rates of RPB1 degradation in cells infected with wt SINV/GFP and SINV/nsP2-683Q/GFP. In correlation with the previously published data, infection of BHK-21 cells with SINV encoding wt nsP2 induced rapid degradation of RPB1, and by 4 h PI only 6% of RPB1 remained (FIG. 3B). In contrast, infection with SINV/nsP2-683Q/GFP did not induce degradation of RPB1, despite both wt and mutant viruses producing essentially similar levels of nsP2 at any time PI.

The previously developed and widely used SINV mutant, SINV/G/GFP, containing the P726G mutation in nsP2, demonstrated reduced cytopathogenicity that correlated not only with a loss of nsP2 nuclear function, but also with lower rates of RNA and virus replication. Thus, to characterize the effect of P683 Q mutation on SINV biology, we infected BHK-21 and NIH 3T3 cells with wt SINV/GFP, SINV/G/GFP and SINV/nsP2-683Q/GFP and compared their clearance and ability to establish persistent infection in these cell types (FIG. 3C). As expected, SINV/GFP rapidly developed complete CPE in both cell types. SINV/G/GFP established persistent infection in BHK-21 cells, which are deficient in development of type I IFN response, and NIH 3T3 cells cleared virus replication within 7 days due to an autocrine effect of the induced type I IFN. In contrast to SINV/G/GFP, infection with SINV/nsP2-683Q/GFP was highly cytopathic despite the ability of the mutant to induce very high levels of type I IFN (see the following sections). As in the case of SINV/GFP that encoded wt nsP2, essentially no cell remained viable after 48 h PI.

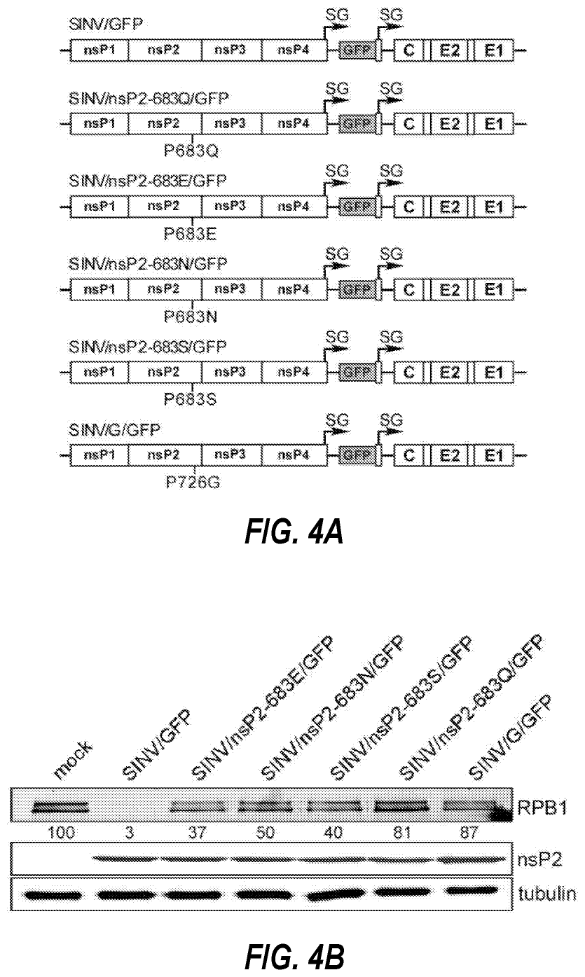

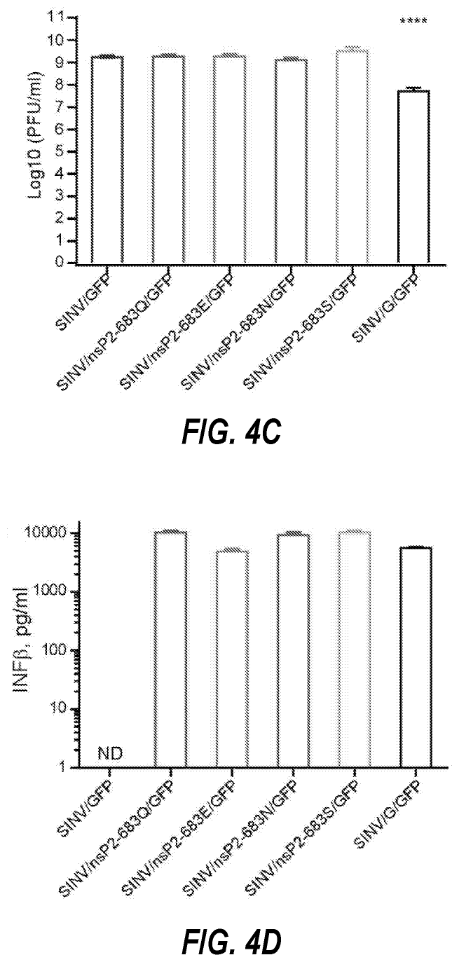

One explanation of the high cytopathogenicity of SINV/nsP2-683Q/GFP could be in its efficient reversion to the wt phenotype, because only a single nucleotide was substituted in the nsP2-coding sequence to generate the mutation. Alternatively, as in the case of P726 mutation the effect the P683 mutation could potentially depend on the substituting amino acid. Thus, next we tested the effects of P683 replacement in SINV nsP2 by different amino acids on virus nuclear functions, replication rates of the variants and efficiency of type I IFN induction. To reduce a possibility of reversion to the wt phenotype, P683E, P683S and P683N mutations were chosen because they required more than one nucleotides to be replaced (FIG. 4A). All of the designed variants were viable. They replicated as efficiently as wt SINV/GFP to more than 50-fold higher titers than the SINV/G/GFP mutant (FIG. 4C). However, similar to SINV/G/GFP, none of the mutant viruses were capable of inducing RPB1 degradation and were all strong inducers of IFN-β in NIH 3T3 cells (FIG. 4B and FIG. 4D). Nevertheless, they remained highly cytopathic.

Thus, the newly designed mutations of P683 of SINV nsP2 abolished nsP2-mediated degradation of RPB1, but had no noticeable effect on virus replication and its ability to cause CPE. These data supported the hypothesis that the mechanism of CPE development by SINV and likely other OW alphaviruses is determined not only by nuclear function of nsP2 in induction of transcriptional shutoff. Other components of virus-host interaction, besides the RPB1 degradation, are capable of CPE induction even in the absence of transcription inhibition.

Mutations in nsP3 reduce cytopathogenicity of SINV/GFP with mutated nsP2. In the above-described experiments with SINV nsP2, we identified amino acid substitutions of P683 that had a deleterious effect on nsP2 nuclear function, but preserved the highly cytopathic phenotype of the virus and its efficient replication. Thus, in the first part of this study, we succeeded in inactivating one of the mechanisms underlying the development of SINV-specific CPE without affecting others. If the hypothesis about redundant involvement of more than one mechanism in CPE development is correct, then selection of noncytopathic SINV-based replicon RNAs could be more efficient using P683 mutants. Since the replicons containing the indicated mutation did not require inactivation of the nsP2 transcription inhibitory function to become noncytopathic, they could more efficiently acquire the mutations that inactivate other nsP-specific mechanisms in CPE development. Identification of such mutations could potentially uncover new aspects of SINV-cell interactions, which are exploited by virus infection in CPE development.

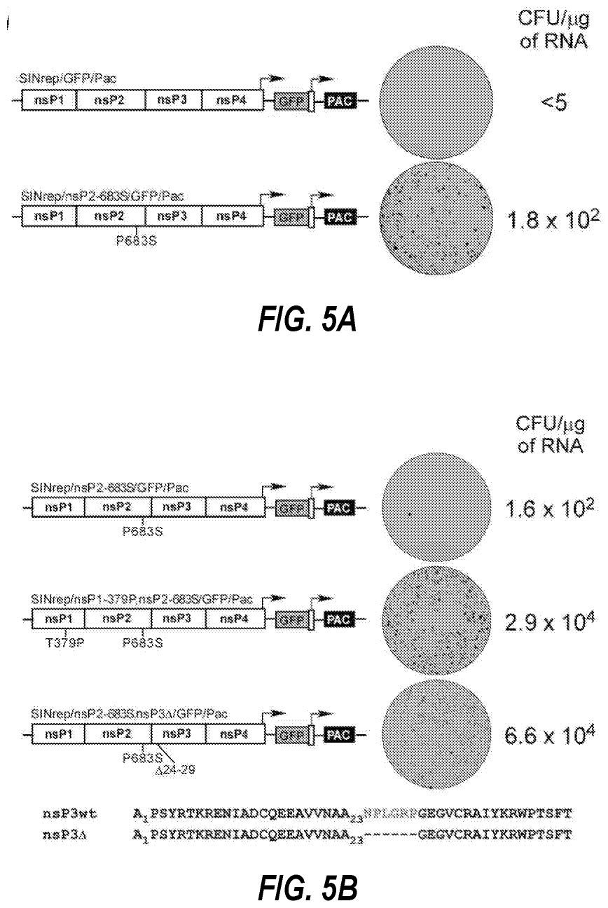

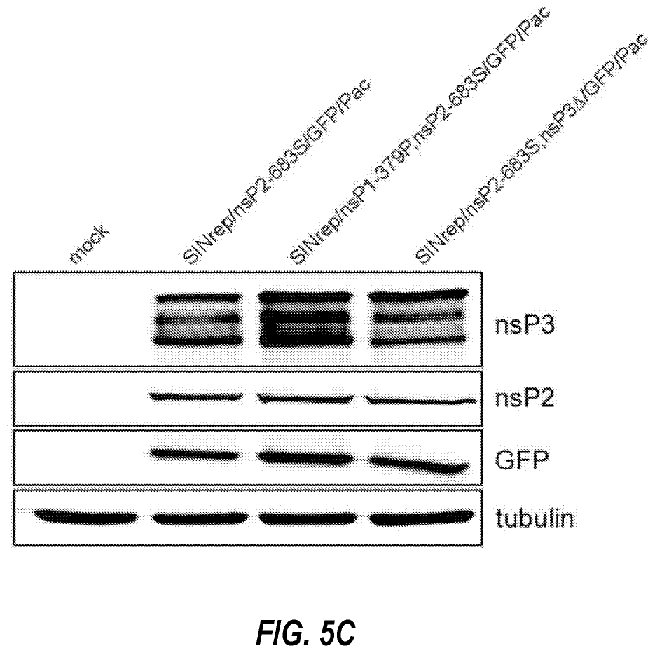

For these new selection steps, we designed SINV replicon, SINrep/nsP2-683S/GFP/Pac. It contained the P683S mutation in nsP2 and encoded GFP and Pac under control of separate subgenomic promoters (FIG. 5A). BHK-21 cells were electroporated with the in vitro synthesized RNAs of this and wt replicons and then subjected to puromycin selection. Wt SINrep/GFP/Pac produced less than 5 colonies of Pur^{R} cells per μg of electroporated RNA. The mutant replicon produced colonies at two orders of magnitude higher efficiency (FIG. 5A). This suggested that a wider range of spontaneous mutations could lead to the development of a noncytopathic phenotype, when the nuclear functions of nsP2 had already been inactivated. We randomly selected two colonies of Pur^{R} cells demonstrating high levels of GFP expression and sequenced the nonstructural genes of the corresponding persisting replicons. In addition to the pre-existing P683S mutation in nsP2, one of them contained another mutation in nsP1, T379P, and in the second replicon, 6 aa in the N-terminus of nsP3 were deleted (A24-29). To confirm the negative effects of these additional changes on cytopathogenicity of replicons, the mutations were introduced into the original SINrep/nsP2-683 S/GFP/Pac construct, and the in vitro-synthesized RNAs were electroporated into BHK-21 cells (FIG. 5B). The new constructs produced more than 10⁴ colonies per μg of transfected RNA. This efficiency of colony formation was similar to that of the previously described SINrep/Pac replicon with nsP2 containing P726L amino acid substitution that had deleterious effects on both nuclear and RC-specific functions of the latter protein. Western blot analysis confirmed that the parental and new replicons expressed similar levels of nsP2 and nsP3 (FIG. 5C), and no abnormalities in polyprotein processing were detected.

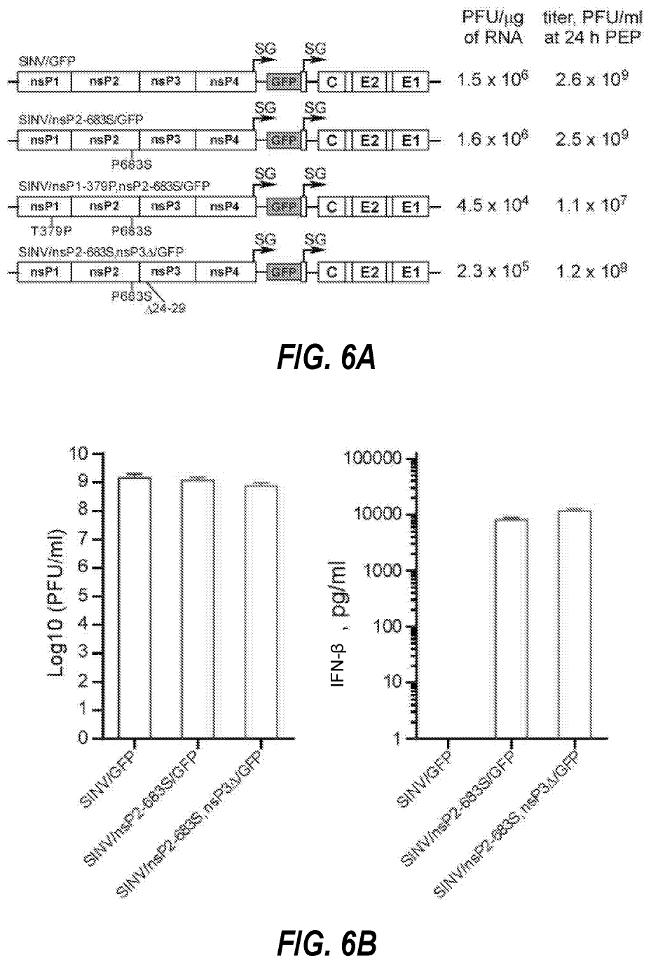

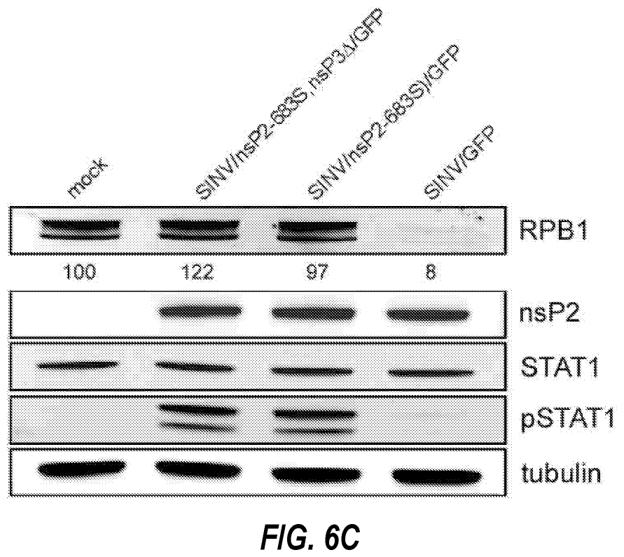

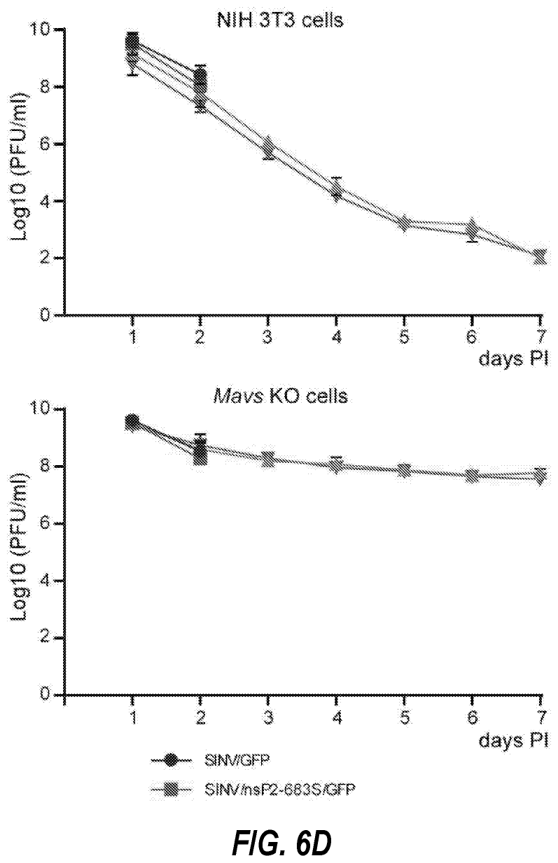

Next, we introduced the identified nsP1- and nsP3-specific mutations into cDNA encoding the infectious viral genome, namely SINV/nsP2-683S/GFP. For yet unclear reason, the nsP1-specific mutation had a strong negative effect on the infectivity of the in vitro-synthesized RNA and the rates of infectious virus release (FIG. 6A). The infectivity of SINV/nsP2-683S,nsP3Δ/GFP RNA was also noticeably lower (about 6 times) compared to SINV/GFP, but the detected decrease was not as strong as when additional adaptive mutations are required for viability. Infectious titers of the harvested stocks of SINV/nsP2-683S,nsP3Δ/GFP were essentially the same as those of SINV/nsP2-683S/GFP and SINV/GFP.

The following experiments in NIH 3T3 cells demonstrated that the double nsP2+nsP3 mutant was capable of efficient replication in this cell line and its titers were essentially the same at both early (8 h) and late (18 h) times PI (FIG. 6B and data not shown) as those of the parental wt SINV/GFP and single SINV/nsP2-683S/GFP mutant. The double mutant efficiently produced nsP2 and did not induce degradation of RPB1 (FIG. 6C). Accordingly, infection with SINV/nsP2-683S/nsP3Δ/GFP stimulated INF-13 release and phosphorylation of STAT1 (FIG. 6B and FIG. 6C). Most importantly, the double mutant was dramatically less cytopathic than SINV/GFP and SINV/nsP2-683S/GFP variants (FIG. 6D). NIH 3T3 cells that have no defects in type I IFN production and signaling, were able to stop replication of the double mutant and clear the infection. However, it was able to persistently replicate in Mavs KO NIH 3T3 cells, which were no longer able to induce IFN-β release in response to virus replication. Thus, the short N-terminal deletion in nsP3 affected another mechanism(s) of SINV-specific CPE development without affecting virus replication rates.

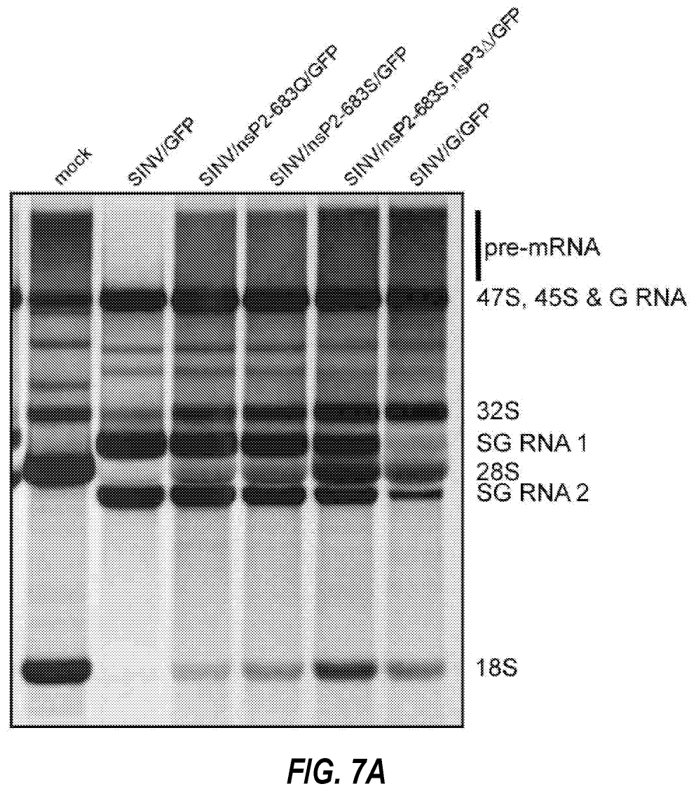

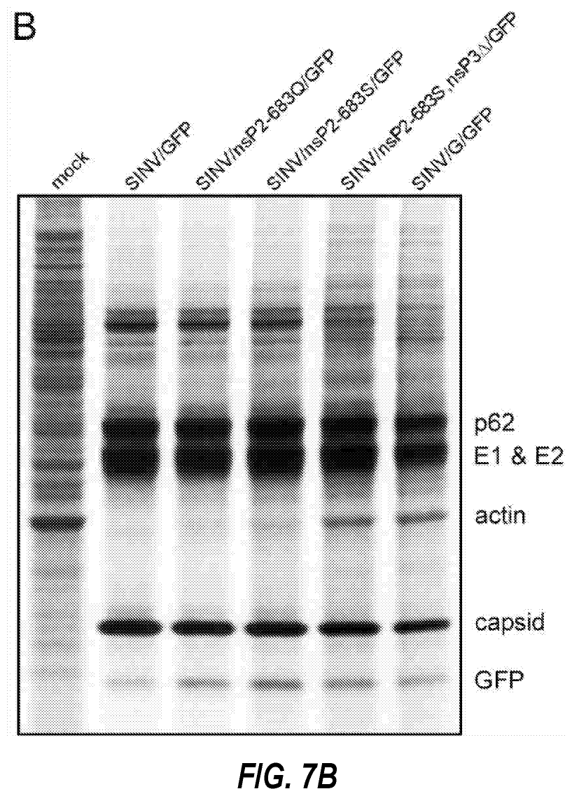

Mutations in SINV nsP2 and nsP3 affect the development of transcriptional and translational shutoffs, respectively. In prior studies, we demonstrated that SINV infection in vertebrate cells rapidly inhibits cellular transcription and translation through independent mechanisms. The transcriptional shutoff is caused by RPB1 degradation, and the translational shutoff is mediated by both PKR-dependent and poorly characterized PKR-independent mechanisms. To evaluate the effects of the newly developed mutants on these critical, virus-specific modifications of the intracellular environment, we performed metabolic pulse labeling of the synthesized RNAs and proteins in virus-infected cells. As previously reported, wt SINV/GFP induced rapid shutoff of cellular transcription and translation (FIG. 7). Within a few hours post infection, cells began to synthesize only viral RNAs and viral structural proteins. The previously developed SINV/G/GFP mutant produced lower levels of SG RNA.

The synthesis of its genomic RNA was likely also inefficient as was previously shown, but in the absence of ActD in the labeling media of this experiment, this effect was not observable, because the radioactively labeled G RNA and abundant 45S and 47S pre-rRNAs co-migrate as a single band on the agarose gels. SINV/G/GFP-infected cells continued to produce a large amount of pre-mRNA and ribosomal RNA (FIG. 7A). As expected, single nsP2 mutants with mutation at P683 and the double nsP2+nsP3 mutant in particular, were also inefficient in transcription inhibition despite high levels of virus-specific RNA synthesis. In contrast, only double mutant SINV/nsP2-683S,nsP3Δ/GFP was less efficient in its inhibition of cellular translation. The presence of a [³⁵S]-labeled actin band was readily detectable on the gel (FIG. 7B).

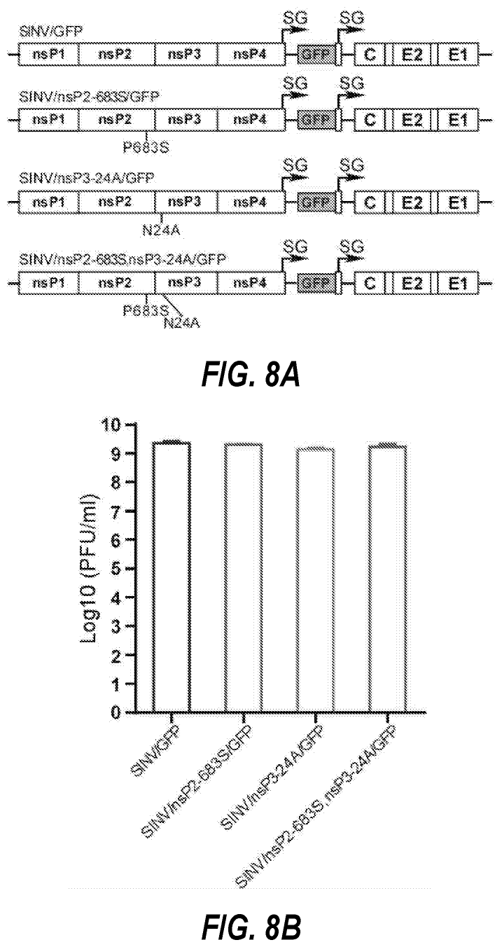

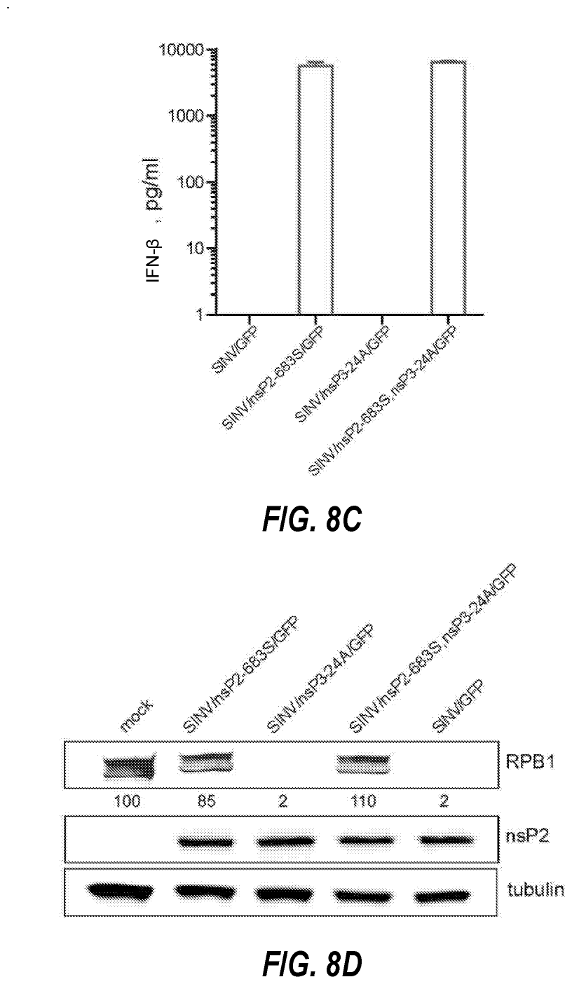

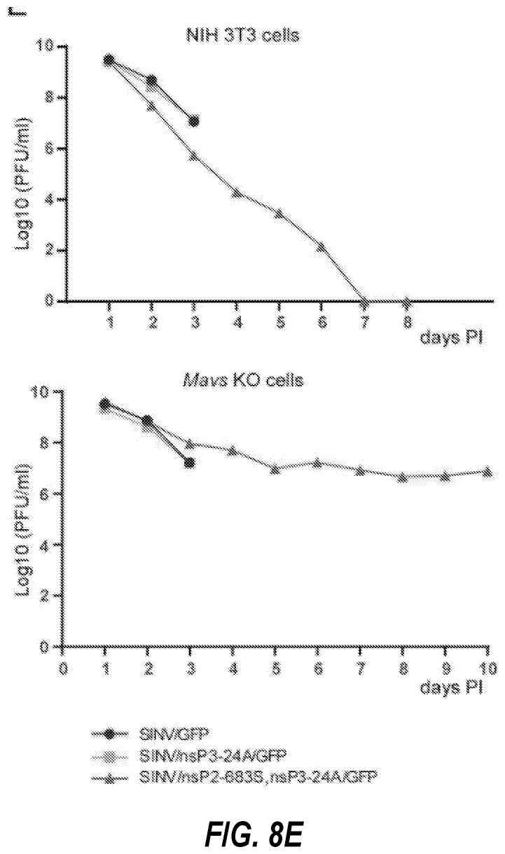

Recently, the N-terminal macrodomain of alphavirus nsP3 was shown to function as mono-ADP-ribosylhydrolase, and a N24A mutation in the CHIKV nsP3 macrodomain abolished this hydrolase activity. N24 was among the amino acids deleted in the selected nsP2-683S,nsP3Δ mutant SINV replicon. This suggested that inhibition of nsP3 mono-ADP-ribosylhydrolase activity may be responsible for further attenuation of SINV variants encoding already mutated nsP2. To experimentally evaluate this possibility, we introduced the N24A mutation into nsP3-coding sequences of SINV/GFP and SINV/nsP2-683S/GFP viral genomes. Both new mutants were viable and replicated to the same titers as their counterparts with wt nsP3 (FIG. 8B). The N24A mutation alone did not make SINV/nsP3-24Δ/GFP variant with the wt nsP2 noncytopathic and a type I IFN inducer, and the latter virus still efficiently induced RPB1 degradation (FIG. 8C and FIG. 8D). However, the double mutant SINV/nsP2-683S,nsP3-24Δ/GFP was an efficient type I IFN inducer, similar to its parental SINV/nsP2(P683S)/GFP. It was also incapable of RPB1 degradation, but lost the highly cytopathic phenotype. SINV/nsP2-683S,nsP3-24Δ/GFP was efficiently cleared from NIH 3T3 cells and readily established persistent infection in Mavs KO cells (FIG. 8E), as we detected with the prototype double nsP2+nsP3 mutant having a deletion of 6 aa in the N-terminus of nsP3 (FIG. 6D). Thus, the effect of N24A point mutation reproduced that of the experimentally selected nsP3-specific deletion and additionally pointed to the possible role of SINV nsP3-specific mono-ADP-ribosylhydrolase activity in the development of translational shutoff and CPE in SINV-infected cells. The detailed characterization of the mechanism of this function is now under investigation.

One of the fundamental characteristics of SINV replication in vertebrate cells is rapid development of CPE. Infected cells usually begin to exhibit morphological changes within 6-10 h PI and lose their integrity and die by 24 h post SINV infection. During this time, the major changes in cell biology may cause the formation of autophagosomes, development of apoptosis, endoplasmic reticulum stress, etc. CPE is determined by a combination of virus-induced changes in cell biology, and the involvement of multiple mechanisms strongly complicates dissection of individual components. In this study, we intended to further understand the molecular basis of the processes that underline development of CPE in SINV-infected cells. Replication process of this virus demonstrates a number of commonalities with those of other OW alphaviruses, and thus SINV represents a good model for studying interactions of other OW alphaviruses with host cells. The most important common characteristics of CPE development by SINV and other OW alphaviruses that we considered in our experiments were as follows.
i) All of the studied OW alphaviruses and their replicons rapidly induce CPE in vertebrate cells.
ii) Replication of all of the OW alphaviruses globally and rapidly inhibits cellular transcription. The transcriptional shutoff is determined by the nuclear fraction of their nsP2 proteins, which induce degradation of the catalytic subunit of cellular DNA-dependent RNA polymerase II, RPB1, and thus, abrogate transcription of cellular mRNAs.
iii) Expression of the wt OW alphavirus nsP2 alone in vertebrate cells also induces inhibition of cellular transcription that is sufficient for inducing cell death and CPE development. However, SINV and SFV nsP2/3 cleavage mutants produce only the unprocessed P23 that remains exclusively in the cytoplasm. These viruses do not induce transcriptional shutoff, but remain highly cytopathic. Their ability to induce CPE suggested the existence of additional virus-induced mechanism(s) of CPE induction.
iv) Importantly, selection of the noncytopathic OW alphavirus replicons was always highly inefficient. Despite relatively low fidelity of both SP6 RNA polymerase, which is used for the in vitro synthesis of replicon genomes, and alphavirus RNA-dependent RNA polymerase, very few colonies of cells containing noncytopathic replicons have been selected. We estimated that ˜1 out of 10⁶ cells that received the in vitro synthesized SINV or SFV replicon were capable of developing a single colony of replicon-containing, drug-resistant cells. Thus, the efficiency of acquiring the noncytopathic phenotype by SINV replicons was 4 orders of magnitude lower than that normally detected during selection of single point mutations, which promote virus replication. This was another indication that more than one virus-specific mechanism is involved in CPE induction, and that very few single point mutations can inactivate more than one process in virus-host interactions and lead to development of a noncytopathic phenotype.
v) To date, all selected noncytopathic SFV, SINV and CHIKV replicons demonstrated highly inefficient RNA replication, suggesting that most likely, besides inactivating nuclear functions of nsP2, the acquired mutations also reduced RNA replication rates and thus, nonspecifically affected the efficiency of CPE induction.

Considering the above-described data, the rationale of this study was to further dissect the fundamental changes in cell biology that ultimately result in CPE development, and to define the roles of SINV nsPs in these processes. SINV nsP2 mutants that are incapable of inducing only the transcriptional shutoff could be a good starting point for identification of other components of CPE. However, to date, only the effects of P726 substitutions in nsP2 have been characterized. They abolished the ability of the latter protein to induce RPB1 degradation and made the virus incapable of CPE induction. However, they also had strong, nonspecific negative effects on RNA and virus replication. Therefore, the P726 mutants could not be used for identification of components of CPE development beyond dissecting the functions of nsP2 in transcription inhibition.

Thus, we first identified another set of attenuating mutations in the nsP2-coding sequence, which made this protein very inefficient in transcription inhibition. The mutated aa P683 and Q684 were located on the surface of the nsP2 molecule close to the previously described P726G substitution (FIG. 1D). However, in contrast to the latter mutation, they had no effect on nsP2 function as a viral replication complex (vRC) component in RNA and virus replication. Similar to wt virus infection, the mutated nsP2 was transported into the nucleus, but did not cause degradation of RPB1. Consequently, the designed viruses became very efficient type I IFN inducers. However, most importantly, they remained cytopathic in all of the tested cell lines of vertebrate origin. Interestingly, the codons of P683 and Q684 were previously identified as the sites of short in-frame sequence insertions into SINV nsP2 by random insertion mutagenesis. Such insertions made nsP2, which was expressed alone, also incapable of CPE induction. The entire set of the insertion sites that affected nsP2 nuclear functions was represented by nsP2 codons 676, 678, 682, 683, 684 and 687. At that time, the effects of the peptide insertions into nsP2 were not further investigated, except to demonstrate that the mutated proteins accumulated in the nuclei. However, the new data suggest a possibility that substitutions of aa 676, 678, 682, 687 and probably others, which are in close proximity to P683 on the protein surface, could also affect interaction of nsP2 with nuclear factors and the ability of this protein to induce CPE.

The selection of nsP2 mutants that no longer exhibited nuclear functions, but did not affect virus replication allowed us to dissect another process involved in CPE development, which was not directly connected to transcription inhibition. At the second step of selection, SINV replicons containing P683S mutation in nsP2 were two orders of magnitude more efficient in formation of Pur^{R} colonies than their wt counterpart. This was an indication that further development of the noncytopathic phenotype could be achieved by numerous additional point mutations in SINV nsP genes. The following experiments were focused on analyzing one of the identified mutations, which led to deletion of six aa in the N-terminus of SINV nsP3. That deletion had no effect on SINV replication or synthesis of virus-specific RNA, but strongly affected development of translational shutoff, which is characteristic of SINV replication in vertebrate cells. The presence of both P683S and A24-26 mutations in nsP2 and nsP3 of SINV replicons caused an additional 100-fold increase in efficiency of Pur^{R} colony formation, indicating that the A24-26 mutation affected a critical mechanism of CPE development in cells containing self-replicating SINV-specific RNAs.

Recently, the N-terminal sequence in the nsP3 macro domain has been suggested to be a part of the nsP3-associated mono-ADP-ribosylhydrolase catalytic site. Thus, we designed an additional viral mutant by substituting a single amino acid, N24A, in the encoded nsP3. Based on the published data, this mutation was expected to inhibit mono-ADP-ribosylhydrolase activity of the macrodomain. The designed double nsP2+nsP3 mutants of SINV that either had the identified deletion of aa 24-29 or the single amino acid N24A substitution, replicated as efficiently as wt virus, but were dramatically less cytopathic. They were either cleared by NIH 3T3 cells, or could persistently replicate in their Mavs KO derivatives. Notably, in the absence of P683S substitution in nsP2, N24A alone had no noticeable effect on either SINV replication rates or the efficiency of transcription inhibition and cytopathogenicity of the virus. This was an additional demonstration that the nsP2-mediated transcriptional shutoff is a critical mechanism of CPE development. The lack of effect of nsP3-specific mutation in the context of wt virus also correlated with the results of our prior study, in which we selected SINV with the insertion of the entire GFP into codon 28 of nsP3. Replication competency of that SINV variant suggested that even strong modifications of this fragment are not lethal for virus replication in vitro. However, data from this study demonstrate that this nsP3 fragment has an important function in SINV-host cell interaction and in the development of translational shutoff in particular. Its role becomes clearly detectable in the absence of another redundant determinant of CPE, namely nsP2-induced transcription inhibition.

Interestingly, instead of having an antiviral effect, inhibition of translation in SINV- and SFV-infected cells is highly beneficial for virus replication. SINV-specific translational shutoff is determined by two mechanisms, one of which is PKR independent, and the second efficiently mediates translational shutoff even in PKR^{-/-} cells. Thus far, the mechanism of PKR independent inhibition of translation has remained unknown, but our new data suggest that nsP3 associated mono-ADP-ribosylhydrolase activity may be a key player in this process. Identification of cellular targets of this nsP3-associated enzymatic activity will be necessary for further understanding of this protein's function.

The additional second site mutation that has been found to strongly reduce cytopathogenicity of the SINV nsP2 mutant has been identified in nsP1 protein. Unlike, the mutation in nsP3 protein, this mutation strongly reduced virus replication. Together with data on previously published attenuated SINV, CHIKV and SFV replicons, which all demonstrated reduced replication rate, this suggest that the nsP3 function in inhibition of translation can be attenuated by reduction of its concentration in infected cells. It further suggests that inhibition of cellular transcription by the nsP3 macrodomain associated mono-ADP-ribosylhydrolase activity is not very efficient, require accumulation of high concentration of nsP3 in cytoplasm and, thus, is detected late in infection.

In summary, the results of this study demonstrate that development of CPE during replication of SINV and probably other OW alphaviruses is determined by multiple mechanisms. One of them is inhibition of transcription, which is mediated by nuclear function(s) of nsP2. The defined mutations in the peptide located on the surface of nsP2 between aa 674-688 can be dispensable for virus replication. However, they prevent virus-induced RPB1 degradation, transcriptional shutoff and make SINV a strong type I IFN inducer. Nevertheless, these mutations are not sufficient for preventing CPE. Further downregulation of SINV cytopathogenicity results from additional mutations in the nsP-coding sequence. The identified mutations in the nsP3 macrodomain, which potentially inhibit its mono-ADP-ribosylhydrolase, made SINV dramatically less cytopathic, but also had no effect on its replication rates. The requirements for acquisition of two independent mutations affecting different aspects of SINV-host interactions provides a plausible explanation for the difficulty of selecting the less cytopathic variants with high levels of RNA and virus replication. These data also open new possibilities for attenuation of the OW alphaviruses and development of efficient and less cytopathic alphavirus expression systems.

Cell cultures. NIH 3T3 cells were obtained from the American Type Culture Collection (Manassas, Va.). BHK-21 cells were kindly provided by Paul Olivo (Washington University, St. Louis, Mo.). These cell lines were maintained at 37° C. in alpha minimum essential medium (□MEM) supplemented with 10% fetal bovine serum (FBS) and vitamins. The Mavs KO cell line has been generated from NIH 3T3 cells by introducing mutation in the second exon of Mays gene using CRISPR technology as we previously described. The sequence for guide nucleic acids were following: GGGAACCGGGACACACTCTG (SEQ ID NO:1) and CAGAGTGTGTCCCGGTTCCC (SEQ ID NO:2). The presence of the modification was confirmed by Sanger sequencing of PCR fragments of targeted region. The absence of MAVS was additionally confirmed by Western blot with anti-MAVS antibodies (sc-365334, Santa Cruz Biotechnology).

Plasmid constructs. Plasmids encoding SINV Toto1101 genomes pSINV/GFP and mutant pSINV/G/GFP and VEEV replicons encoding SINV nsP2 gene, pVEEVrepL/nsP2-GFP/Pac and pVEEVrepL/nsP2-GFP-NLS/Pac were described elsewhere. All plasmids containing cDNAs of mutant replicons and viruses were constructed using standard PCR-based techniques. All mutations were confirmed by Sanger sequencing. The schematic representations of all of the modified genomes are shown in the corresponding figures. Sequences of the plasmids and details of the cloning procedures can be provided upon request.

In vitro RNA transcription and transfection. Plasmids were purified by ultracentrifugation in CsCl gradients. Then they were linearized using unique restriction sites located downstream of the poly(A) sequence. RNAs were synthesized by SP6 RNA polymerase in the presence of a cap analog (New England Biolabs) according to the manufacturer's recommendations (Invitrogen). Aliquots of transcription reactions were used for electroporation without additional purification. Electroporation of BHK-21 cells by in vitro-synthesized viral genomes was performed under previously described conditions. Viruses were harvested at 20-24 h post electroporation. Virus titers were determined by a plaque assay on BHK-21 cells.

SINV nsP2 noncytotoxic mutant selection. BHK-21 cells were electroporated with 5 μg of the in vitro-synthesized VEEV or SINV replicon RNAs pVEEVrepL/nsP2-GFP/Pac or SINVrep/nsP2-683S/GFP/Pac respectively and plated to 100-mm tissue culture dishes in different dilutions. 6 hours post electroporation media was replaced by puromycin containing media in concentration 5 μg/ml. Electroporated cells grew under puromycin selection for 12 days. Developed cell clones were collected and used for TRIzol-based RNA extraction followed by cDNA synthesis using Super Script III reverse transcriptase from Invitrogen according manufactures recommendations. Set of the overlapping primers corresponding to the SINV nsP1, nsP2 or nsP3 sequence was used. PCR products were sequenced by Sanger sequencing.

Analysis of the cytotoxicity of SINV replicons. 5 μg of the in vitro synthesized replicon RNAs SINVrep/nsP2-683S/GFP/Pac, SINVrep/nsP1-379,nsP2-683 S/GFP/Pac and SINVrep/nsP2-683S,nsP3Δ/GFP/Pac, and SINVrep/GFP/Pac were electroporated into BHK-21 cells. 6 hours post electroporation media was replaced by fresh media supplemented with puromycin (10 μg/ml) for the first 5 days and then changed to 5 μg/ml. Survived colonies of the puromycin-resistant cells were fixed with paraformaldehyde 3 to 9 days post electroporation depending on cells grow rate. Colonies were stained with crystal violet and manually counted.

Infectious center assay. To compare infectivities of the viral RNAs, BHK-21 cells were electroporated with 1 μg of the in vitro-synthesized genomic RNAs. Ten-fold dilutions of electroporated cells were seeded in 6-well Costar plates containing subconfluent monolayers of naïve BHK-21 cells. After 2 h of incubation at 37° C., cells were overlaid with agarose supplemented with MEM and 3% FBS. Plaques were stained after 2 days of incubation at 37° C., and RNA infectivity was determined as PFU/μg of transfected RNA.

Analysis of virus replication. Cells were seeded into 35-mm dishes and infected at MOIs indicated in the figure legends. At the indicated times, media were harvested, and virus titers in the samples were determined by plaque assay on BHK-21 cells.

Analysis of the viral persistence. Indicated cell lines were infected at MOI 20, or otherwise stated in the figures, washed with PBS and overlaid with competent medium. Cell media was collected, cells were washed with PBS and media was replaced by fresh every 24 hours for 10 days. Virus titers were estimated by plaque assay on BHK-21 cells as described previously.

IFN-β measurements. Media collected at the indicated time points and pH was stabilized by HEPES. Concentration of the IFN-β in the media was estimated by VeriKine Mouse interferon Beta ELISA kit according to the manufacturer's recommendations (PBL Assay Science).

Western Blotting. 4-12% NuPAGE gels (Invitrogen) were used for separation of the equal amounts of the proteins. Samples were transferred to 0.42 μm nitrocellulose membrane form Amersham and blocked in 5% Blotting grade (BioRad). Overnight incubation with primary antibodies was followed by incubation with infrared dye-labeled secondary antibodies. Membranes were scanned on the Odyssey imager (LI-COR Biosciences). Quantitative analysis of the bands was performed by the imager software. The band intensities were normalized to intensity of tubulin band. The following primary antibodies were used for Western blotting: tubulin (rat mAb, UAB core facility), rabbit polyclonal antibodies against SINV nsP3 (custom), mouse monoclonal antibodies against alphavirus nsP2 (custom), STAT1 (rabbit mAb, Epitomics), pSTAT1 (mouse mAb, pY701, BD Transduction Laboratories), RPB1 (8wG16, Covance; 4H8, Active Motif or F12, Santa Cruz Biotechnology).

Analysis of protein synthesis. NIH3T3 cells were seeded into p60 plates 1×10⁶ cells/plate and infected by SINV/GFP, SINV/G/GFP, SINV/nsP2-683Q/GFP, SINV/nsP2-683S/GFP or SIN/nsP-6832S, nsP3Δ/GFP at a MOI:20. 6 hours post infection media was replaced by Dulbeco modified Eagle medium (DMEM) lacking methionine, supplemented with 0.1% FBS and 20 μCi of [³⁵S]methionine/ml. After 30 minutes of incubation cells were washed, scraped, resuspended in standard cell lysis buffer and equal amounts of the protein were loaded on 10% SDS-PAGE. Gel was vacuum dried and autoradiographed.

Confocal microscopy. Cells were seeded in 8-well Ibidi chambers (5×10³/well) and incubated overnight at 37° C. They were then infected with the packaged replicons indicated in the figures. At the times post infection indicated in the figure legends, cells were fixed with 4% paraformaldehyde (PFA) for 15 minutes, permeabilized and stained with Alexa Fluor 555 phalloidin and Hoechst dye. The image stacks of 6 optical sections were acquired on a Zeiss LSM700 confocal microscope with a 63× 1.4NA PlanApochromat oil objective. The image images were assembled using Imaris software (Bitplane AG).

### Example 2: Mutations in nsP2-Specific Peptide Make Chikungunya Virus Noncytopathic without Affecting Viral Replication Rates

The Alphavirus genus in the Togaviridae family contains a variety of human and animal pathogens, which are widely distributed on all continents. Based on the geographical circulation, they can be divided into the New World (NW) and the Old World (OW) alphaviruses. However, the recent spread of the OW chikungunya virus (CHIKV) to Central and South Americas and Caribbean islands suggested that such division does not any longer reflect current viral distribution and renders some flexibility. In natural conditions, alphaviruses are transmitted by mosquito vectors between amplifying vertebrate hosts. In vertebrates, they induce diseases of different clinical symptoms. The NW alphaviruses induce highly debilitating disease that results in meningoencephalitis with a frequent lethal outcome or neurological sequelae. The OW representatives, exemplified by Sindbis virus (SINV), Semliki Forest virus (SFV) and CHIKV, are generally less pathogenic than those prevalent in the New World, and their human-associated diseases are usually limited to rash, fever, and arthritis. However, within recent years, CHIKV became a viral pathogen of particular concern because of its spread to the new areas and the severity of symptoms induced in infected humans. In many cases, CHIKV-specific polyarthritis is characterized by excruciating joint pain that can continue for years.

As for other alphaviruses, CHIKV genome (G RNA) is represented by a single-stranded RNA of positive polarity of ˜11.5 kb in length. It mimics the structure of cellular messenger RNAs in that it contains Cap at the 5′ terminus and a poly(A) tail at the 3′ terminus. G RNA encodes only a handful of proteins. The nonstructural viral proteins (nsPs) are translated directly from G RNAs as polyprotein precursors P123 or P1234. Together with a CHIKV-specific set of host factors, they form replication complexes (RCs) that initially contains P123+nsP4. At later times post-infection (PI), after complete polyprotein processing by nsP2-associated protease activity, the mature RCs include individual nsP1, nsP2, nsP3, and nsP4. These RCs are efficient in the synthesis of viral G RNA and subgenomic (SG) RNA, which serves as a template for translation of viral structural proteins. After a few steps of processing, the latter proteins form infectious G RNA-containing viral particles.

Despite recent progress in understanding functions of nsPs in viral replication and other aspects of virus interactions with host cells, many processes mediated by viral nonstructural proteins remain to be characterized. Alphavirus nsP2 has numerous known enzymatic activities, which include its function as a helicase and during viral RNA synthesis, protease function in ns polyprotein processing and RNA 5′triphosphatase activity during capping of viral G and SG RNAs. nsP2 can also acquire mutations that compensate negative effects of the modifications introduced into the promoter elements of viral genomes, into nsP3 or in capsid protein. Another critically important function of nsP2, which is specific only for the OW alphaviruses, including CHIKV, is its ability to accumulate in the nuclei of vertebrate cells, where nsP2 induces polyubiquitination of the catalytic subunit of cellular DNA-dependent RNA polymerase II, RPB1. This ultimately leads to proteasomal degradation of RPB1 and abrogates messenger and ribosomal RNA synthesis. The resulting global transcriptional shutoff makes cell incapable of activating transcription-dependent antiviral response and cell signaling, and ultimately induces cell death. Expression of nsP2 alone without viral replication is also highly cytotoxic for vertebrate cells, suggesting its critical function in viral pathogenesis on molecular and cellular levels.

The previous studies showed that the carboxy terminal S-adenosyl-L-methionine (SAM)-dependent RNA methyltransferase-like (SAM MTase-like) domain of SINV and SFV nsP2 is not directly involved in protease and helicase functions of the protein. However, defined point mutations had a deleterious effect on the ability of nsP2 to induce transcriptional shutoff. They made viral mutants and/or mutated alphavirus replicons that expressed no structural proteins, dramatically less cytopathic than their wild-type (wt) counterparts and capable of inducing the antiviral response in the infected cells. These results strongly suggested that the SAM MTase-like domain of the OW alphavirus nsP2 plays a critical role in the nuclear function of the protein. However, the above-mentioned SINV and SFV nsP2 mutations also affected RNA and viral replication rates, therefore their effects on virus cytopathogenicity were difficult to unambiguously interpret.

Development of attenuated constructs encoding CHIKV replication complex (RC) with mutated nsP2 was found to be more challenging. Previously, the protocol that was based on the selection of noncytopathic replicons has been successfully applied to other OW alphaviruses, such as SINV and SFV. These defective, self-replicating viral genomes (replicons) had all of the structural genes in the SG RNA replaced by a selectable marker. Despite lacking the structural genes, SINV- and SFV-based replicons remained cytopathic. However, rare spontaneous mutations in the nsP2-coding sequence could produce a noncytopathic phenotype and make them capable of persistent replication in some vertebrate cells lines that had defects in type I IFN response. In contrast, a similar selection approach was less successful when applied to CHIKV replicons, and multiple mutations were required for making it less cytopathic. These mutations also had a deleterious effect on RNA replication rates, and thus were not applicable for development of stable, replication-competent viruses.

Alphaviruses with altered nuclear functions represent important systems for further understanding the molecular mechanism of their pathogenesis and virus-host interactions. Thus, if remain capable of efficient replication, such CHIKV mutants could open an opportunity for generating new vaccine candidates. The results of our previous and present study strongly suggest that a short highly variable peptide (V peptide) in CHIKV SAM MTase-like domain, which is located on the surface of nsP2 between aa 673 and 678, may play a critical role(s) in the protein's nuclear functions. After applying a number of approaches, we generated a variety of CHIKV variants and CHIKV replicons with mutated V peptide, which sustained high levels of replication, but no longer exhibited transcription inhibitory functions. The designed noncytopathic CHIKV replicons can be used for screening of antiviral drugs, and viral mutants can be further developed as potential vaccine candidates against CHIKV infection.

Cell cultures. NIH 3T3 cells were obtained from the American Type Culture Collection (Manassas, Va.). BHK-21 cells were kindly provided by Paul Olivo (Washington University, St. Louis, Mo.). These cell lines were maintained at 37° C. in alpha minimum essential medium (αMEM) supplemented with 10% fetal bovine serum (FBS) and vitamins. The MAVS KO NIH 3T3 cell line was generated using CRISPR nuclease vector plasmid according to the manufacturer's instructions (Invitrogen) as described elsewhere. Cell clones were initially analyzed in terms of target protein expression, and then KO of both alleles was confirmed by sequencing the targeted fragment in the cell genome.

Plasmid constructs. The original plasmids containing the infectious cDNAs of the attenuated strain CHIKV 181/25 were from the University of Texas Medical Branch, Galveston, Tex.). The derivative of this infectious cDNA clone that encodes GFP under control of subgenomic promoter, CHIKV/GFP, was described elsewhere. pCHIKrep/Pac and pCHIKrep/GFP/Pac were designed using standard PCR-based techniques. Mutations into V peptide-coding sequence of replicons were introduced by standard PCR. Library of replicons with randomized V peptide was made using gene block with randomized corresponding nucleotide sequence. All of the introduced modifications were confirmed by sequencing. Sequences of the plasmids and details of the cloning procedures can be provided upon request.

In vitro RNA transcription and transfection. Plasmids were purified by ultracentrifugation in CsCl gradients. Then they were linearized using Not I restriction sites located downstream of the poly(A) of viral and replicon genomes. RNAs were synthesized by SP6 RNA polymerase in the presence of a Cap analog (New England Biolabs). Quality and concentrations of the synthesized RNAs were tested by agarose gel electrophoresis, and aliquots of the transcription reactions were used for electroporation without additional RNA purification. Electroporations of BHK-21 cells by in vitro-synthesized virus-specific RNAs were performed under previously described conditions. Viruses were harvested at 20-24 h post electroporation, and titers were determined by plaque assay on BHK-21.

Infectious center assay (ICA). To compare infectivities of the in vitro-synthesized RNA, their equal amounts were electroporated into BHK-21 cells. Ten-fold dilutions of electroporated cells were seeded in 6-well Costar plates containing subconfluent monolayers of naïve BHK-21 cells. After 2 h of incubation at 37° C., cells were overlaid with agarose supplemented with MEM and 3% FBS. Plaques were stained with crystal violet after 3 days of incubation at 37° C., and RNA infectivity was determined as PFU/μg of electroporated RNA.

Analysis of virus replication. In standard experiments, 5×10⁵ cells in 6-well Costar plates were infected with recombinant CHIKV at an MOIs indicated in the figure legends in 200 μl of phosphate-buffered saline (PBS) supplemented with 1% FBS. After 1-h-long virus adsorption, the inoculum was replaced by complete media, and cells were incubated at 37° C. At the time points indicated in figures, media were replaced, and viral titers in the harvested samples were determined by plaque assay on BHK-21 cells. In the analyses of viral clearance or persistent replication, cells were spitted upon reaching confluency.

IFN-β measurement. NIH 3T3 cells were infected with recombinant viruses as described in the figure legends. In the harvested samples, and the pH in the media was stabilized by adding HEPES buffer pH 7.5 to 0.01 M. Concentrations of IFN-β were measured with the VeriKine Mouse Interferon Beta ELISA Kit (PBL InterferonSource) according to the manufacturer's recommendations.

Western blotting. Equal amounts of protein lysates were separated on a 4-12% gradient NuPAGE gel (Invitrogen). After protein transfer, the membranes were incubated with primary antibodies, followed by incubation with infrared dye-labeled secondary antibodies. For imaging and quantitative analysis, membranes were scanned on the Odyssey imager (LI-COR).

Analysis of RNA analysis. NIH 3T3 cells were infected with recombinant viruses at an MOI of 20 PFU/cell. Viral and cellular RNAs were metabolically labeled between 4 and 8 h PI in 0.8 ml of complete media supplemented with [³H]uridine (20 μCi/ml). RNAs were isolated from the cells by TRizol reagent as recommended by the manufacturer (Invitrogen), and then denatured with glyoxal in dimethyl sulfoxide as described elsewhere. RNAs were analyzed by agarose gel electrophoresis in 0.01 M Na-phosphate buffer pH 7.0. Gels were impregnated with 2,5-diphenyloxazol (PPO) and used for autofluorography.

Analysis of protein synthesis. 5×10⁵ NIH 3T3 cells were six-well Costar plates were infected with recombinant CHIKV variants at an MOI of 20 PFU/cell for 1 h. After 6 h of incubation at 37° C., cells were washed with PBS and then incubated for 30 min at 37° C. in Dulbecco's modified Eagle's medium lacking methionine, supplemented with 0.1% FBS and 20 mCi of [³⁵S]methionine/ml. Cells were dissolved in standard loading buffer for protein electrophoresis, and equal amounts of lysates were analyzed by electrophoresis in 10% NuPAGE gels followed by autoradiography.

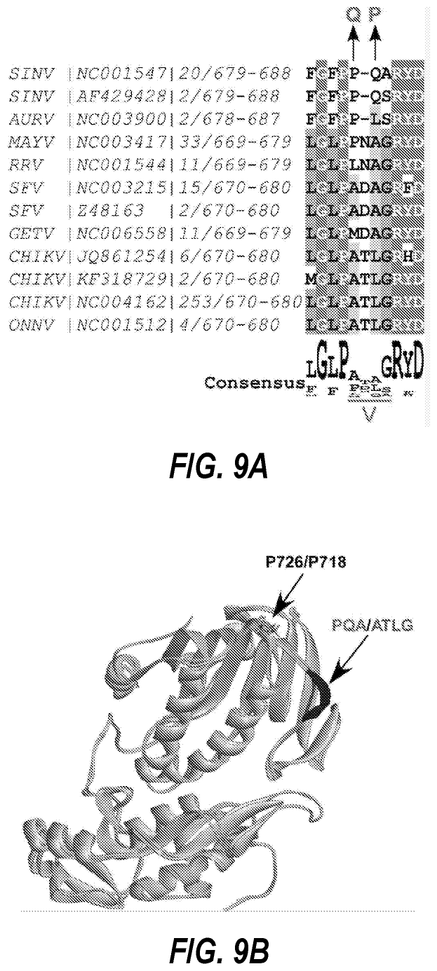

Introduction of nsP2 mutations identified in SINV to CHIKV nsP2 makes virus less cytopathic. In this study, we initially made an attempt to apply our recent SINV-based data about the molecular mechanism of inhibition of cellular transcription for the development of attenuated mutants of CHIKV. We have selected a variety of SINV variants that contained mutations at P683 or Q684 in the carboxy terminal SOM domain of nsP2. These mutations strongly affected the ability of SINV nsP2 to induce RPB1 degradation and, consequently, made virus incapable of efficient inhibition of antiviral response. Importantly, these mutations did not affect SINV replication rates in the tested cell lines of vertebrate origin. The alignment of the available aa sequences of the OW alphavirus nsP2 proteins demonstrated that the SINV-specific P683 and Q684 mutations were in the small peptide (V peptide) located between two conserved aa sequences. V peptide itself exhibited a very low level of identity between the members of SINV and SFV serocomplexes (FIG. 9A) and showed variability in the overall length between 3 and 4 aa. Structural alignment of the V peptide-containing carboxy-terminal domains of SINV and CHIKV nsP2 proteins demonstrated that for both viruses, it is located at the protein surface in close proximity to P726 (SINV), which was also previously shown to be critical for protein function in the degradation of RPB1 subunit of RNA polymerase II.

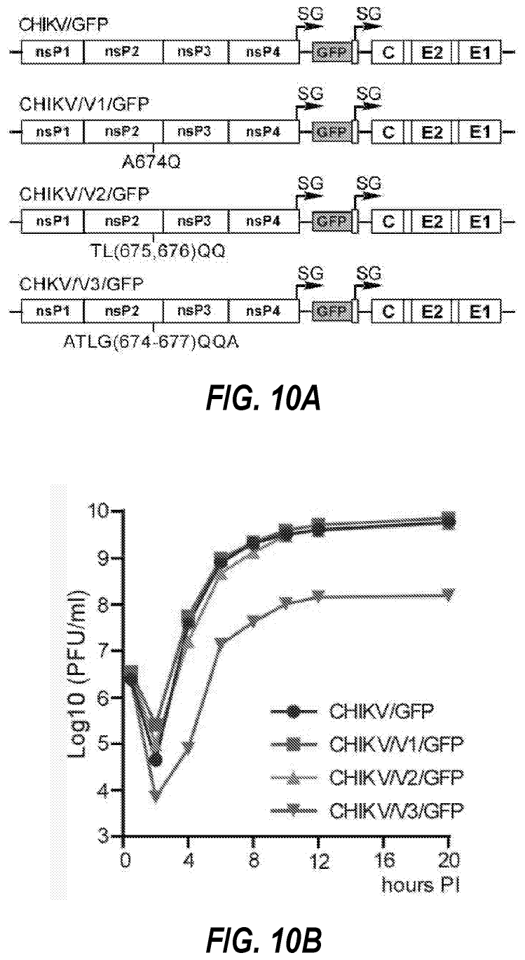

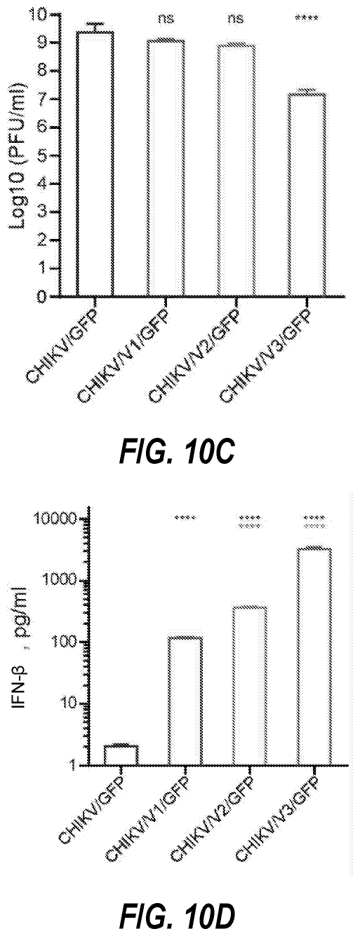

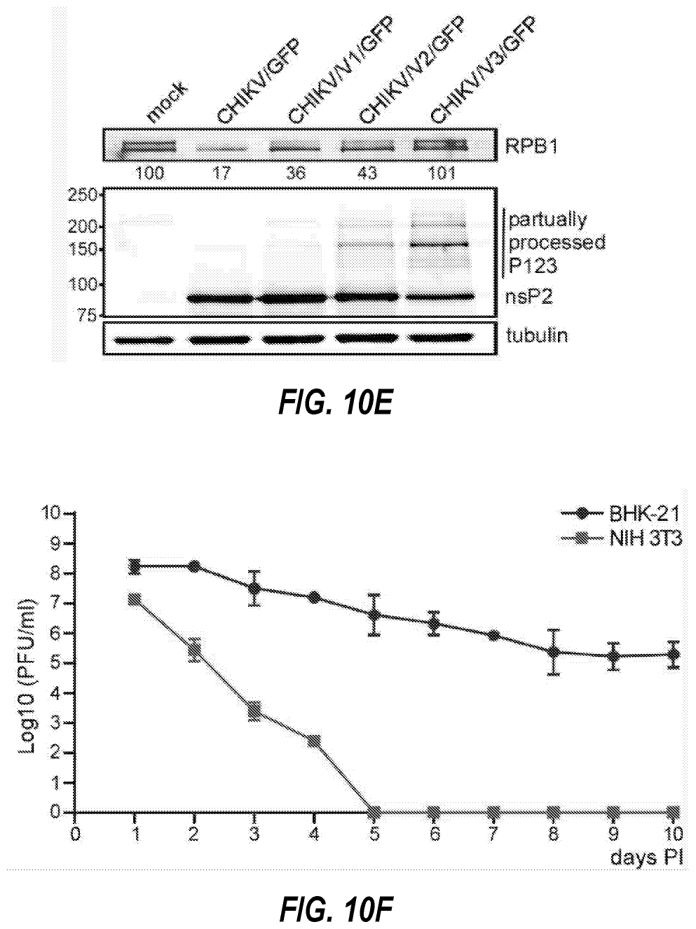

Based on the above data, it was reasonable to expect that similar to SINV, some mutations in the V peptide of CHIKV nsP2 may abolish protein's nuclear function(s). Therefore, we focused our efforts on introducing mutations into this short sequence in CHIKV genome. The first approach was based on replacement of one or more aa in the nsP2-specific V peptide of CHIKV 181/25 strain by the combinations of aa found in the corresponding peptides of attenuated SINV nsP2 mutants. Accordingly, CHIKV/V1/GFP contained QTLG instead of ATLG, CHIKV/V2/GFP encoded AQQG, and CHIKV/V3/GFP had the entire ATLG replaced by QQA (FIG. 10A). Since at least some of the new mutants were expected to be less cytopathic, all of them and control CHIKV/GFP were designed to encode GFP gene under control of the subgenomic promoter. GFP expression was used to monitor the levels of viral replication and infection spread in cultured cells.

The in vitro-synthesized RNAs were equally infectious, and within 8 h post electroporation, for all of the samples, equal numbers of cells were GFP-positive (data not shown). Titers of CHIKV/V1/GFP and CHIKV/V2/GFP in the stocks harvested at 24 h post electroporation, were the same as those of control CHIKV/GFP, but CHIKV/V3/GFP replication resulted in almost 100-fold lower infectious titers below 10⁸ PFU/ml. CHIKV/V3/GFP mutant also did not develop cytopathic effect (CPE) in BHK-21 cells at any time post electroporation. However, it was still capable of producing small plaques in these cells under agarose cover in the presence of low levels of FBS. NIH 3T3 cells, which in contrast to BHK-21 are fully competent in type I interferon (IFN) production and signaling, produced CHIKV/V1/GFP and CHIKV/V2/GFP as efficiently as control CHIKV/GFP (FIG. 10B). In contrast, at any times PI, titers of the mutant with the replaced V peptide, CHIKVN3/GFP, were 50-100-fold lower. In NIH 3T3 cells, CHIKV/GFP, which encoded wt nsP2, induced IFN-β at the limit of detection, while replication of all three nsP2 mutants led to IFN-β accumulation at readily detectable levels (FIG. 10C). CHIKV/V3/GFP was the most efficient in IFN-β induction, and this result correlated with its inability to induce RPB1 degradation (FIG. 10D). At 8 h PI of NIH 3T3 cells, CHIKV/V1/GFP and CHIKVN2/GFP caused partial degradation of RPB1, but not as efficiently as parental CHIKV/GFP (FIG. 10D), while in CHIKV/V3/GFP-infected cells, RPB1 remained intact.

To compare the ability of the designed mutants to induce CPE, NIH 3T3 and BHK-21 cells were infected with all of the generated viruses at the MOI of 10 PFU/cell, and virus replication was analyzed for 10 days. CHIKV/GFP, CHIKV/V1/GFP, and CHIKV/V2/GFP caused CPE in both cell lines within 48 h PI. In contrast, CHIKVN3/GFP infection was noncytopathic. The latter mutant was cleared from NIH 3T3 cells within 5 days PI by the autocrine effect of the released type I IFN (FIG. 10E). In BHK-21 cells, the latter mutant established persistent infection.

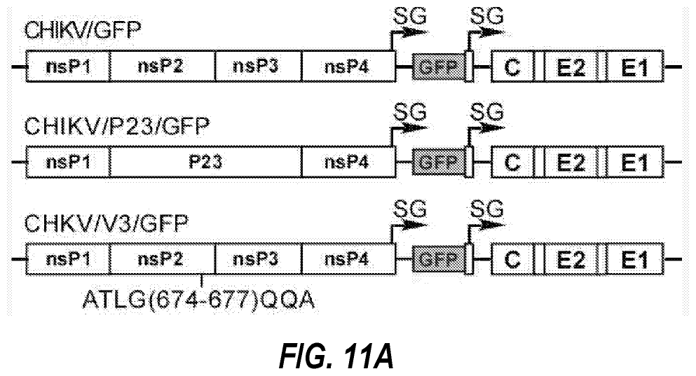

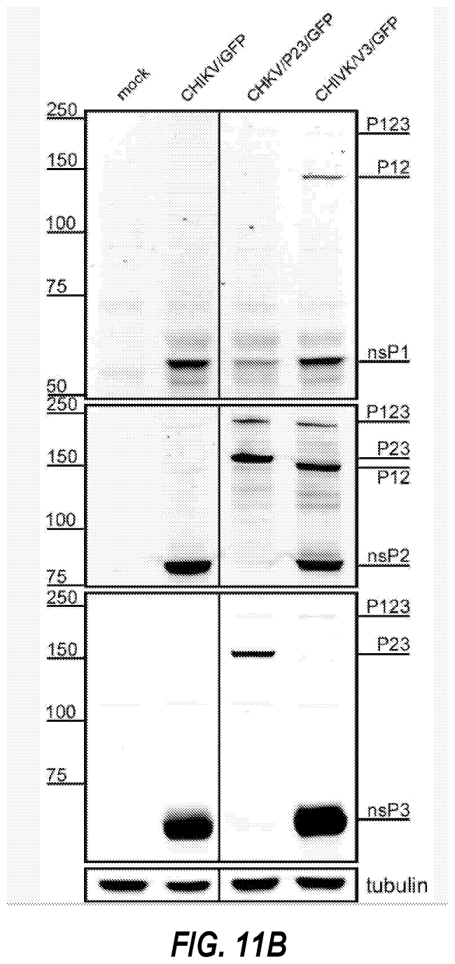

Mutations in V peptide of CHIKV nsP2 affect P12 processing. Western blot analysis of nsP2 accumulation in infected cells revealed that the introduced nsP2-specific mutations in CHIKV/V2/GFP and CHIKV/V3/GFP variants altered processing of ns P123/P1234 polyproteins (FIG. 10E). In addition to nsP2, the high molecular weight proteins were readily detectable by nsP2-specific Abs. However, since the products of partial processing, P12 and P23 have similar sizes, it remained unclear, which particular step of cleavage was affected. To distinguish between the possibilities, NIH 3T3 cells were infected with CHIKV/GFP, CHIKV/V3/GFP and an additional control virus CHIKV/P23/GFP at MOI 20, and at 8 h PI, the nonstructural proteins were analyzed by Western blot using nsP1-, nsP2- and nsP3-specific Abs (FIG. 11). CHIKV/P23/GFP contained a mutation in nsP2\hat{ }3 cleavage site and was applied as a P23-producing virus. At this time PI, the CHIKV/GFP-infected cells contained only individual nsPs. In CHIKV/P23/GFP-infected cells, we detected nsP1, P23, and P123 but not nsP2. Cells infected with CHIKV/V3/GFP, in contrast, exhibited the presence of a readily detectable fraction of P123 and P12, but not P23. Thus, processing of the entire ns polyprotein of CHIKV/V3/GFP mutant and 1\hat{ }2 cleavage site, in particular, were strongly affected. This alteration of nsP processing was likely at least partially responsible for lower viral replication rates.

Taken together, the data indicated that mutations in the V peptide (₆₇₄ATLG₆₇₇) of CHIKV nsP2 had negative effects on the ability of the virus to interfere with activation of cellular antiviral response. For all three newly designed variants, the mutations affected the ability of CHIKV nsP2 to induce RPB1 degradation and ultimately, the efficiency of transcriptional shutoff and INF-β release. However, CHIKVN2/GFP and particularly the most attenuated CHIKV/V3/GFP mutant also exhibited alterations in P12 processing, and in addition, CHIKV/V3/GFP replicated less efficiently than wt virus.

Selection of efficiently replicating CHIKV nsP2 mutants that lack transcription inhibitory functions. Taken together, the results of the above experiments demonstrated that V peptide plays a critical role in the nuclear inhibitory functions on CHIKV nsP2 and viral cytopathogenicity. However, the introduced mutations did not generate CHIKV variants to combining wt levels of replication and high levels of type I IFN induction indicating the loss of nuclear function of nsP2. They also demonstrated alterations in ns polyprotein processing. Nevertheless, lack of defects in the P123 processing in the previously designed noncytopathic SINV, which contained P683Q mutation in V peptide, suggested that development of CHIKV mutants with altered nuclear, but not other nsP2 functions may be feasible. However, it could require designing and testing of a wide variety of variants with mutated V peptide. Therefore, in order to generate and test a wide collection of mutants, we applied an alternative approach.

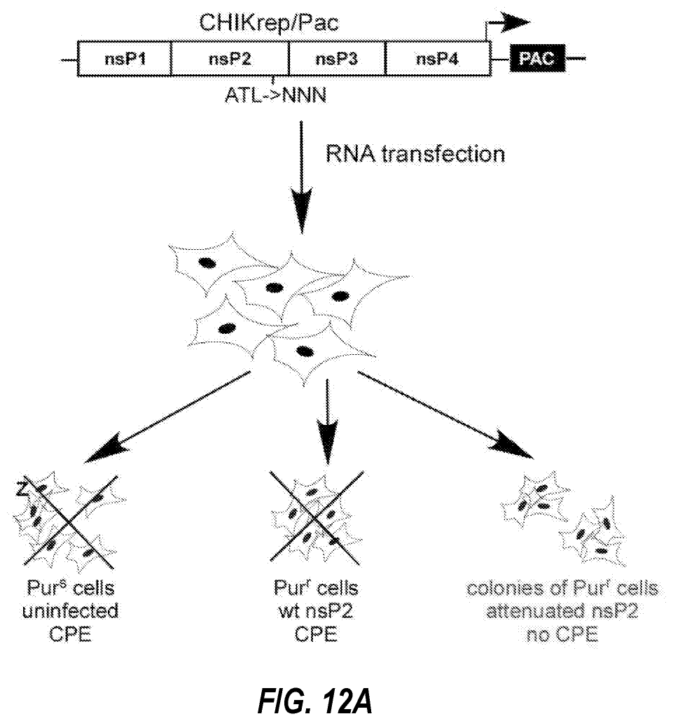

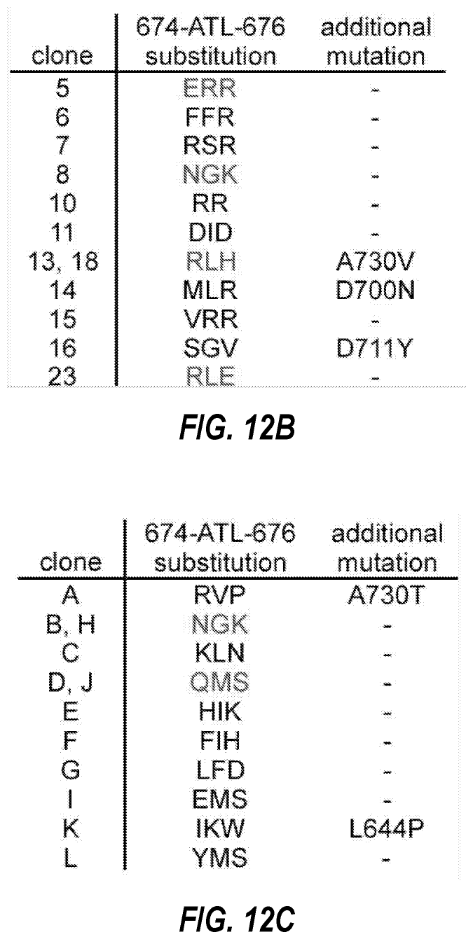

CHIKV replicon (CHIKrep/Pac), which encoded puromycin acetyltransferase (Pac) gene under control of the subgenomic promoter (FIG. 12A), was used as a starting construct. Its replication in BHK-21 cells was highly cytopathic. In repeated experiments, after transfections of the in vitro-synthesized CHIKrep/Pac RNA into BHK-21 cells followed by puromycin selection, no colonies of Pur^{R} cells were observed. The sequential introduction of point mutations into V peptide, followed by analysis of their effect on cytopathogenicity of the replicon or virus could be endless. Therefore, we replaced codons encoding V peptide (₆₇₄ATL₆₇₆) (FIG. 9A) in CHIKrep/Pac nsP2 with the randomized nucleotide sequence (FIG. 12A). Plasmids from the library of ˜10⁴ clones of E. coli were used for the synthesis of the replicon genomes and the entire RNA pool was next electroporated into BHK-21 cells. Following selection with puromycin resulted in ˜400 colonies of Pur^{R} cells. The untransfected cells and those containing nonviable replicons died due to translational arrest caused by puromycin. Most of the cells have likely received the cytopathic variants of the replicon and died because of the nsP2-induced, CPE-causing transcriptional shutoff. The residual survived cells contained no longer cytopathic CHIKrep/Pac mutants, which were probably unable to induce RPB1 degradation. We randomly selected 24 colonies and expanded them in the presence of a higher concentration of puromycin. After this additional step, 12 colonies were also randomly selected for sequencing of the mutated nsP2 gene. The identified sequences of the V peptide and additional spontaneous mutations, found in some variants are presented in FIG. 12B.

In additional selection protocol, the total population of selected Pur^{R} clones was further passaged as a pool in the presence of puromycin for three weeks. This procedure was aimed at the selection of the most efficiently growing cells, which likely contained more attenuated replicons. Then total RNA was isolated from the cell pool, the nsP2 sequence was amplified by PCR and cloned into the plasmids. Insertions from the plasmids of 12 randomly selected clones were sequenced and the identified variants of the V peptide and additional mutations are presented in FIG. 12C.

Analysis of the identified mutant V peptides (FIG. 12B and FIG. 12C) revealed that i) many of them contained positively charged amino acids, arginine or lysine, in the first or third position of the peptide; ii) several noncytopathic replicons contained additional mutations in the nsP2 gene, and they were also located in the carboxy-terminal SOM domain, which had been previously implicated in playing critical role(s) in SINV and SFV RNA replication; iii) three aa sequences (RLH, NGK, and QMS) were repeatedly detected in the selected mutants. iv) The NGK sequence was detected using both selection protocols.

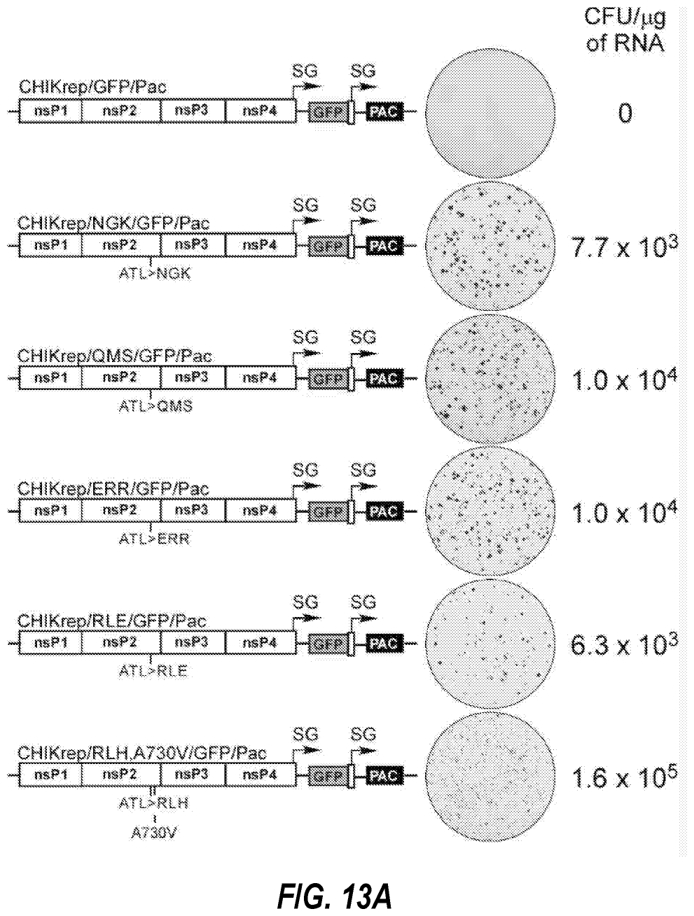

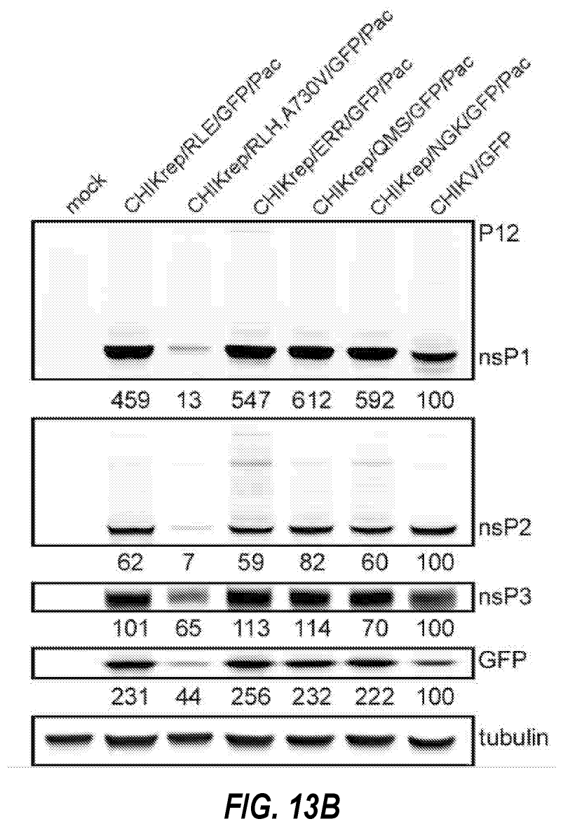

Analysis of the effects of identified mutations on cytopathogenicity of CHIKV replicon. Sequences encoding some of the peptides (indicated in red in FIG. 12B and FIG. 12C) were cloned to replace the original ₆₇₄ATL₆₇₆-encoding fragment into another replicon, CHIKrep/GFP/Pac, that encoded both GFP and Pac genes under control of subgenomic promoters (FIG. 13A). In these experiments, GFP expression was used as a means of indirect evaluation of G RNA replication and synthesis of SG RNA levels. Equal amounts of the in vitro-synthesized RNAs of the designed replicons and CHIKrep/GFP/Pac, encoding wt nsP2, were electroporated into BHK-21 cells, and colonies of Pur^{R} cells were selected. As expected, no colonies were formed upon transfection of CHIKrep/GFP/Pac, despite within 24-48 h post transfection, essentially all of the cells were GFP-positive indicating RNA replication. In contrast, cells transfected with mutated replicons produced large numbers of colonies of Pur^{R}, GFP-positive cells. The replicon with the additional mutation in nsP2, CHIKrep/RLH,A730V/GFP/Pac, was the most efficient in colony formation, indicating the least cytopathic phenotype. However, based on GFP fluorescence (data not shown) and Western blot analysis of GFP, nsP1, nsP2 and nsP3 expression (FIG. 13B) in the replicon-transfected BHK-21 cells, the latter mutant replicated less inefficiently. Importantly, all mutant replicons demonstrated either very small or no defect in P123 processing (FIG. 13B).

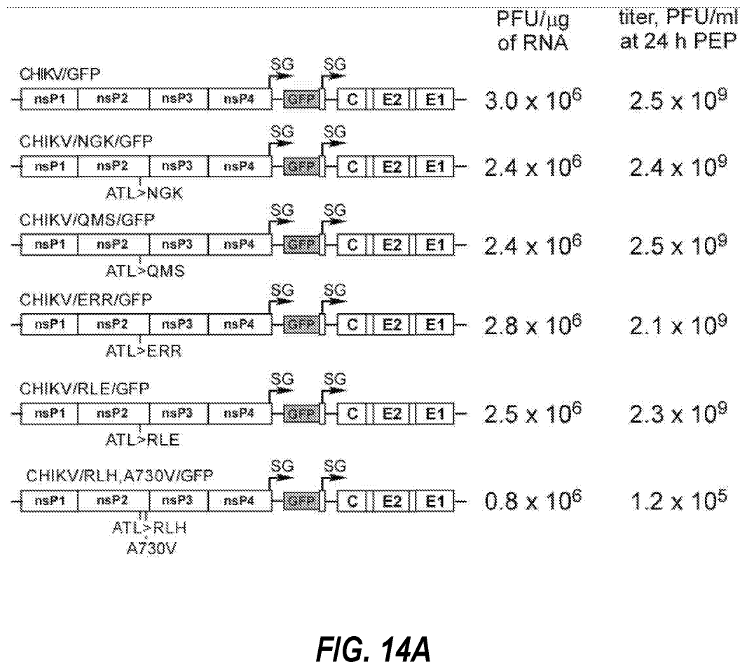

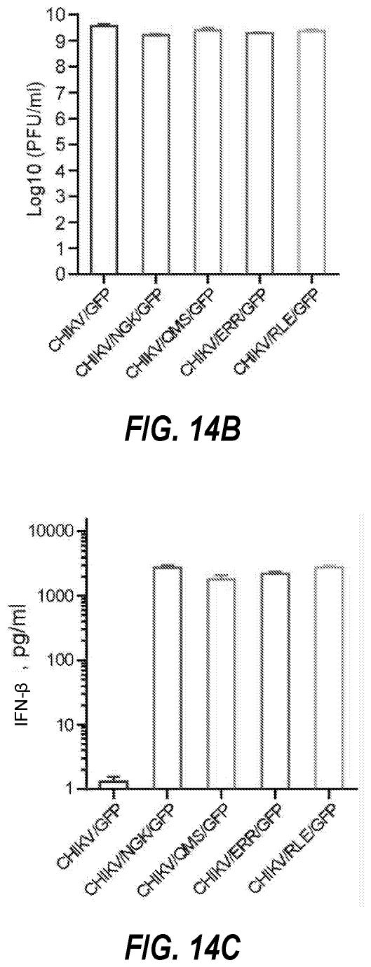

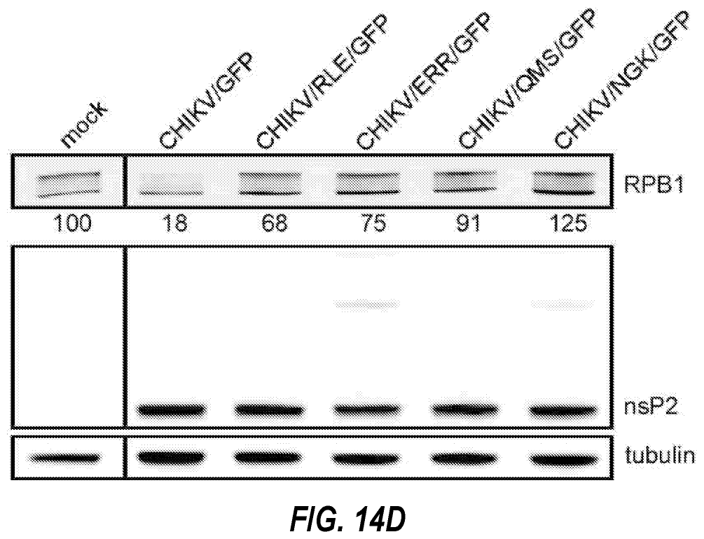

The selected mutations in the V peptide affected the ability of CHIKV to induce transcriptional shutoff. All of the designed replicons were attenuated in terms of their ability to induce CPE, but it remained unclear whether the same mutations had any effect on replication of the virus. To test this, we next cloned the above-presented, mutated V peptide sequences into CHIKV genome (FIG. 14A). In the infectious center assay, the in vitro-synthesized RNAs of the mutants and CHIKV/GFP, encoding wt V peptide, demonstrated similar infectivity (FIG. 14A). This was suggestive that the designed constructs did not require additional adaptive mutations for their viability. Four mutants were able to replicate in electroporated BHK-21 cells to the same titers as CHIKV/GFP. However, the final titers in the electroporation-derived stocks of the double mutant CHIKrep/RLH,A730V/GFP were reproducibly lower, and in standard plaque assay, its plaques were almost undetectable. Further analysis of the effect of the second site, nsP2-specific A730V mutation demonstrated that it had a strong negative effect on CHIKV RNA replication. Therefore, experiments with the CHIKV/RLH, A730V/GFP mutant were discontinued.

Next, NIH 3T3 cells were infected with the mutants and parental CHIKV/GFP at the same MOI, and both viral titers and IFN-β release were compared at 22 h PI. No significant differences in viral titers between wt CHIKV/GFP and the mutants were detected (FIG. 14B). However, in this murine cell line, in contrast to parental CHIKV/GFP and previously developed constructs with mutated V peptide (FIG. 10D), the designed mutants were very potent IFN-β inducers. As we expected, they were inefficient in degradation of RPB1, and this provided a plausible explanation for detected high levels of IFN-β induction (FIG. 14D).

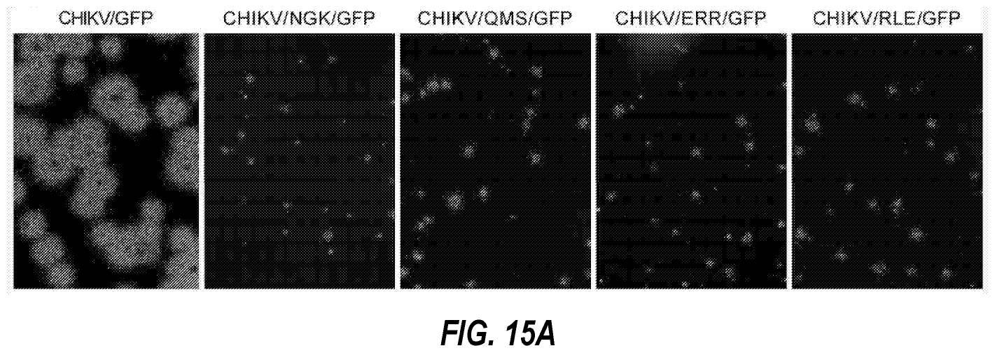

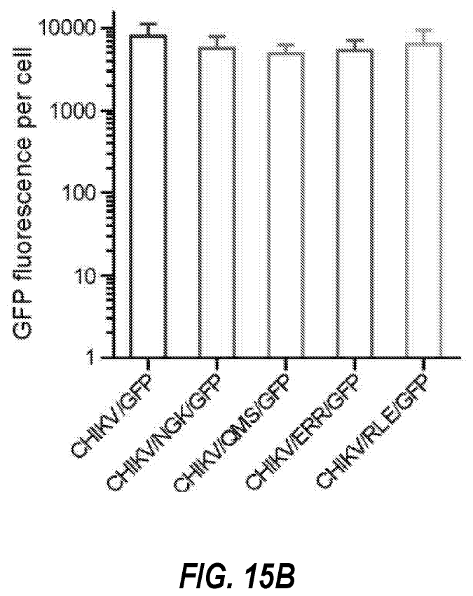

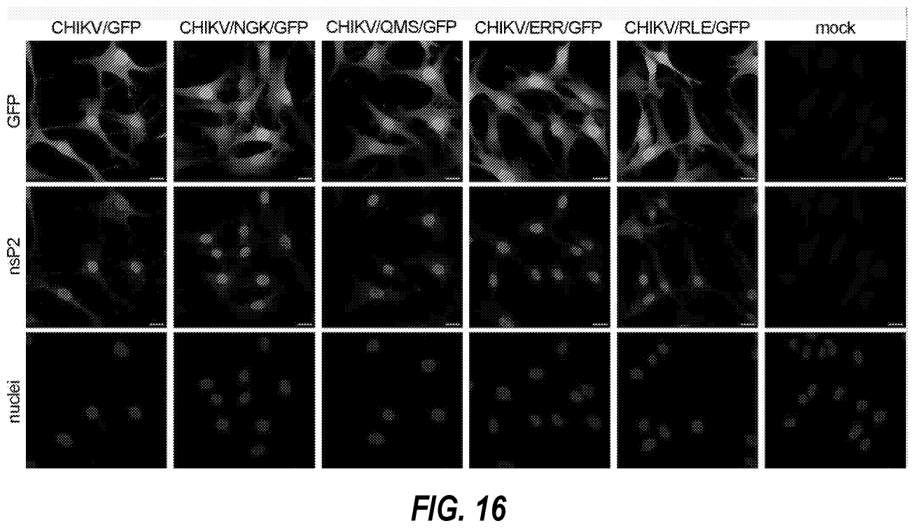

All four variants were able to rapidly spread in BHK-21 cells and formed plaques in this cell line under agarose cover supplemented with a low concentration of FBS (data not shown). However, in contrast to CHIKV/GFP, they could not spread and produce plaques in murine cells that were fully competent in type I IFN induction and signaling. FIG. 15A presents foci of GFP-positive cells formed under agarose by the indicated mutants and CHIKV/GFP in the NIH 3T3 cells at 24 h PI. Additional comparative analysis of these images did not reveal significant differences in the intensity of GFP fluorescence per cell (FIG. 15B), and thus, the inability to spread was caused by IFN-β release from the primarily infected cells. This was an additional indication of the critical role of CHIKV nsP2-specific nuclear inhibitory function in the development of spreading viral infection.

In additional experiments, we tested whether the introduced mutations changed intracellular distribution of CHIKV nsP2 compared to wt counterpart, or the mutations affected nsP2 nuclear function. Cells were infected with CHIKV/GFP and above-described mutants and at 6 h PI, stained with nsP2-specific Abs. The results presented in FIG. 16 demonstrate that none of the mutations had detectable effect on intracellular distribution of the protein. As in case of CHIKV/GFP infection, mutated nsP2 accumulated to high level in the nuclei, but lost an ability to induce degradation of RPB1 (FIG. 14D), to downregulate cellular transcription and ultimately, to inhibit activation of the antiviral response (FIG. 14C).

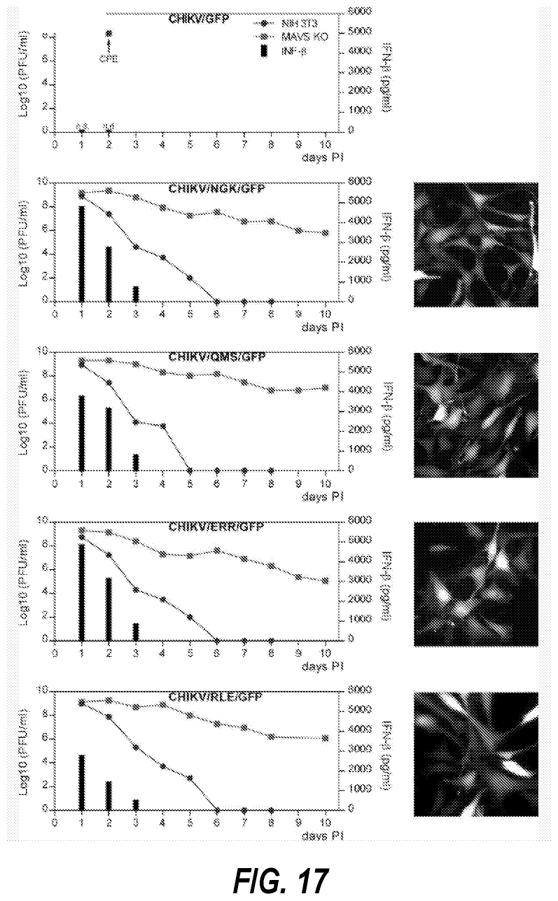

Cytopathogenicity of the designed mutants and their ability to establish persistent replication were further evaluated as follows. The NIH 3T3 and MAVS KO cell lines were infected by CHIKV/GFP and its nsP2 mutants at an MOI of 20 PFU/cell. Within 8 h PI, all of the cells became GFP-positive. Within the next 2 days, CHIKV/GFP caused complete CPE in both cell lines. However, replication of the designed nsP2 mutants was noncytopathic, albeit the CHIKrep/RLE/GFP mutant noticeably affected cell growth (data not shown). In response to mutants' replication, NIH 3T3 cells released high levels of IFN-β, cleared the infection (FIG. 17). Within 5-6 days PI, they became GFP-negative, and infectious virus was no longer present in the media. In contrast, MAVS KO NIH 3T3 cells continued to express GFP (FIG. 17) and released the infectious viruses for the entire 10-days-long duration of the experiment.

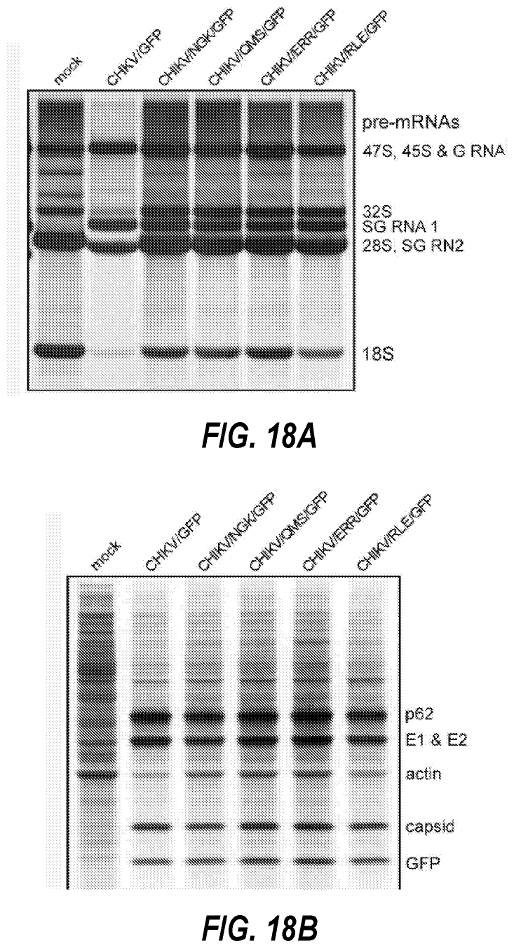

CHIKV nsP2 mutants do not interfere with transcription of cellular messenger and rRNAs. To further characterize the effects of CHIKV nsP2 mutants on cellular transcription and translation, NIH 3T3 cells were infected at the same MOI with the designed variants and parental CHIKV/GFP. Cellular and viral RNAs were metabolically labeled with [³H]uridine in the absence of Actinomycin D (ActD) between 4 and 8 h PI. RNAs were analyzed by agarose gel electrophoresis in denaturing conditions as described herein. CHIKV/GFP efficiently inhibited synthesis of both pre-mRNAs and rRNAs (FIG. 18A). In contrast, cells infected with the designed nsP2 mutants continued to efficiently produce pre-mRNAs and rRNAs.

Protein synthesis was analyzed at 6 h PI with the indicated viruses. In contrast to previously published data demonstrating the robust development of translational shutoff in SINV- and SFV-infected cells, CHIKV inhibited translation of cellular proteins less efficiently (FIG. 18B). The above-described experiments had demonstrated the noncytopathic phenotype of the nsP2 mutants in the NIH 3T3 cells. Therefore, the noticeably lower level of synthesis of cellular proteins was likely sufficient for cell survival during the first 2 days PI, when the most efficient virus replication takes place in both NIH 3T3 and their MAYS KO derivatives.

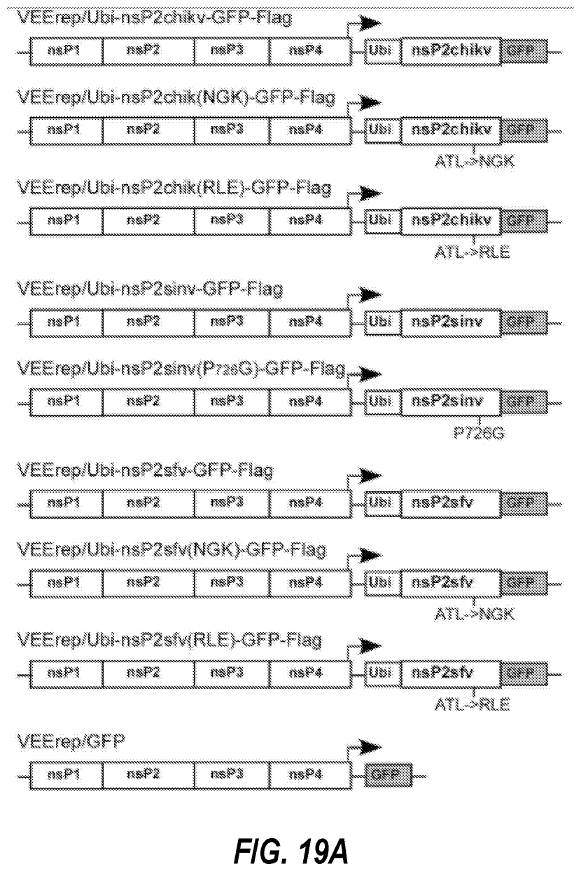

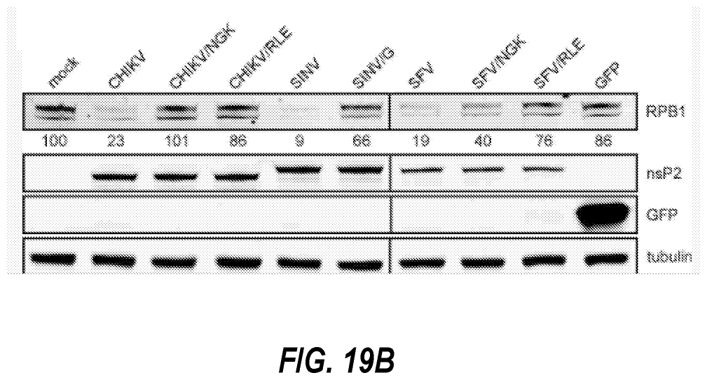

SFV nsP2 with mutations in V peptide does not induce RPB1 degradation. The results of this and previous studies demonstrated that V peptide plays a critical role in nuclear functions of SINV and CHIKV. They suggested that this highly variable peptide might similarly function in other OW alphaviruses during regulating cellular transcription by nsP2 protein. To test this possibility, we cloned wt SFV nsP2 and its variant with mutated V peptide into VEEV replicon as GFP fusion under control of the subgenomic promoter (FIG. 19A) and packaged both replicons into infectious virions. NIH 3T3 cells were infected at the same MOI with VEEV replicons expressing indicated fusions of wt or mutant SINV, CHIKV and SFV nsP2. At 8 h PI, cells expressing wt nsP2 proteins demonstrated essentially the same levels of RPB1 degradation. The mutated versions of nsP2 became less efficient in this degradation, and the effect depended on the V peptide sequence used (FIG. 19B) These results supported our hypothesis that V peptide represents an excellent target for modifications aimed at development attenuated OW alphavirus mutants that are less efficient in interfering with the antiviral response and thus, are stronger inducers of type I IFN than wt viruses.

The alphavirus genome encodes a small set of structural and nonstructural proteins that facilitate replication of viral genome and its packaging into released viral particles. The same proteins also modify the entire biology of the cell. The resulting changes promote more efficient viral replication while downregulating induction of cellular antiviral response that can activate cell signaling and interfere with the infection spread. Alphaviruses have developed distinct means of inhibiting the development of the antiviral state. The New World (NW) alphaviruses, such as Venezuelan (VEEV) and eastern (EEEV) equine encephalitis viruses, utilize their capsid protein to block the function of nuclear pores and nucleocytoplasmic traffic. This, in turn, leads to rapid transcriptional shutoff, causes CPE, and makes cells incapable of both inducing cytokine expression and activating interferon-stimulated genes (ISGs). Capsid proteins of the OW alphaviruses, such as CHIKV, SINV and SFV, do not exhibit this function, but instead, their nsP2 proteins mediate degradation of RPB1, the catalytic subunit of cellular DNA-dependent RNA polymerase II. Expression of the OW alphavirus nsP2 alone is highly cytotoxic and is sufficient for causing transcriptional shutoff Thus, transcription inhibitory function is a common characteristic of both OW and NW alphaviruses and is one of the critical contributors to their abilities to cause CPE in cultured cells. It is also an important determinant of alphavirus pathogenesis. Alterations of VEEV TC-83 capsid protein-specific nuclear functions without affecting viral replication rates, made such VEEV mutants i) dramatically less cytopathic and ii) capable of persistent replication in murine cells, which were deficient in type I IFN signaling. iii) In the cells competent in IFN signaling, these mutants were very efficient inducers of type I IFN, which rapidly activated ISGs in yet uninfected cells and eliminated replicating mutants from those already infected. iv) The designed VEEV mutants also became less pathogenic in vivo. In another line of research, chimeric VEE/CHIKV or EEE/CHIKV viruses encoding the combination of the OW alphavirus-specific capsid and the NW alphavirus-specific nsP2, both of which have no transcription inhibitory functions, were unable to cause CPE in vitro and any detectable disease in vivo. These experiments have demonstrated that the alterations of the viral nuclear-specific inhibitory functions can be used as an important means of alphavirus attenuation.

Development of the OW alphavirus mutants, and CHIKV in particular, that encode nsP2 deficient in induction of transcriptional shutoff, is more challenging than designing modified NW alphavirus with mutated capsid protein. The OW alphavirus nsP2 has a complicated, multidomain structure and numerous functions in replication of viral RNA. Therefore, even small modifications, such as point mutations, usually affect a variety of critical processes in viral replication and alter the viability of the mutants. Nevertheless, a few earlier studies have identified a small set of point mutations in SINV and SFV nsP2 that made viral replicons less cytopathic and capable of persistent replication in vertebrate cell lines defective in type I IFN production/signaling. However, these attenuating point mutations also strongly affected RNA replication rates. Their presence in the viral genome opened an opportunity for viral evolution and usually led to the rapid appearance of more efficiently replicating true and pseudorevertants. Another complication was that the mutations selected in the context of nsP2 of one virus species, such as SINV, had different or no effect on cytopathogenicity of other alphavirus representatives, such as CHIKV. In contrast to SINV and SFV replicons, which required single point mutations in their nsP2 for transformation to noncytopathic phenotype, CHIKV nsP2 had to be strongly mutated to produce similar phenotype. The latter extensive modifications had deleterious effects on replication of CHIKV-specific RNAs. Therefore, such nsP2 mutants could not be used for development of attenuated and at the same time, efficiently replicating viruses.

In the recent study, we have identified additional mutations in SINV nsP2, which had strong negative effects on viral transcription inhibitory functions and cytopathogenicity without affecting its replication rates. These mutations were located in a small highly variable peptide, V peptide, on the surface of the carboxy terminal SAM MTase-like domain of nsP2. Therefore, in the initial experiments, it was reasonable to introduce them into the V peptide of CHIKV and explore their effects on CHIKV's ability to cause CPE and inhibit cellular transcription. The results presented in FIG. 10 demonstrated that such modifications had detectable negative effects on CHIKV nsP2 nuclear functions. However, development of more attenuated and efficiently replicating viral mutants would require testing of thousands of variants having mutated V peptide.

The use of a library of CHIKV variants having randomized V peptide opened an opportunity to evaluate a wide range of the mutations in terms of their ability to produce the noncytopathic phenotype in self-replicating CHIKV RNAs (FIG. 12). Application of this library-based approach led to a selection of hundreds of colonies of Pur^{R} cells, in which replication of CHIKV-specific RNA and expression of nsPs had no deleterious effect on cell viability. Following sequencing of the persistently replicating CHIKV replicons identified a wide range of the V peptide sequences that made RNA replication no longer cytopathic for BHK-21 cells. After replacement of the wt V peptide-encoding sequence in CHIKV replicon (CHIKrep/GFP/Pac) by those identified, the mutated constructs became capable of developing persistent and noncytopathic replication. Their efficiency to form Pur^{R} colonies depended on the particular introduced sequence and, to some extent, on the level of viral RNA replication. The least efficiently replicating CHIKrep/RLH,A730V/GFP/Pac produced the highest numbers of colonies. However, other noncytopathic replicons demonstrated higher levels of replication that could be sufficient for supporting replication of corresponding infectious viruses. Indeed, CHIKV variants encoding V peptide of more efficiently replicating constructs were viable. They did not require additional adaptive mutations and replicated to the same titers as CHIKV/GFP expressing wt nsP2. Most importantly, despite retaining efficient replication, in contrast to parental CHIKV/GFP, they all became very potent IFN-β inducers. Accordingly, they could not develop a spreading infection in the cells competent in type I IFN production and signaling. All of these mutants were cleared from already infected murine cells within 5-6 days PI by autocrine IFN-β signaling that led to ISG activation. Because of being noncytopathic, they readily developed persistent infection in NIH 3T3 MAVS KO cells, which were unable to mount type I IFN response. The designed viruses did not cause degradation of RPB1 and inhibition of transcription of cellular messenger and ribosomal RNAs (FIG. 14 and FIG. 18). They also did not decrease cellular translation to the levels inconsistent with cell viability. The new viruses represent the first example of CHIKV mutants that demonstrate essentially wt levels of replication in cell culture and lack nuclear functions, despite in the infected cells, their mutated nsP2 proteins accumulated in the nuclei as efficiently as wt nsP2 (FIG. 16). These results were different from those previously published for V motif SINV mutant with mutations in the V motif (REF). The SINV mutant had remained highly cytopathic and required the additional mutations in the nsP3 ADP-ribose binding domain to prevent inhibition of cellular transcription.

Taken together, the data point to the role of the nsP2 SAM MTase-like domain in CHIKV interaction with host cells and in the function of viral replication complexes. The previous studies demonstrated that presence of the latter domain is a requirement for nsP2-specific helicase and protease activities, and mutations can make SINV, SFV, and CHIKV either very poorly replicating or nonviable. On the other hand, this domain plays also a critical role in the nuclear function of nsP2, albeit the mechanism remains insufficiently understood. In the case of NW alphaviruses, such as VEEV and EEEV, nsP2 has no nuclear functions. Since the NW alphavirus nsP2 is not transported to the nucleus, it remains unclear whether the nuclear function is completely lost or lack of it is a result of a change of nsP2 compartmentalization during viral replication. In any scenario, despite having a high level of identity with the OW alphavirus-specific counterparts, the NW alphavirus SAM MTase-like nsP2 domain became incapable of inhibiting the antiviral response but retained functions in the enzymatic activities of the protein. This appears to be a logical step in nsP2 evolution because expression of VEEV and EEEV capsid proteins in the chimeric SIN/VEEV or SIN/EEEV viruses completely blocks translocation of SINV nsP2 into the nuclei.

In summary, this study resulted in the identification of small peptide on the surface of the OW alphavirus CHIKV nsP2 that plays a critical role in determining the nuclear function of this nonstructural protein. The selected amino acid sequences that replaced the original wt V peptide had no negative effect on CHIKV replication in the tested cell types. However, they had a deleterious effect on the transcription inhibitory functions of CHIKV nsP2 and made the corresponding CHIKV variants dramatically less cytopathic Inhibition of the innate immune response is a fundamental characteristic of CHIKV replication and, as in case of many other viruses, is likely a major component of viral pathogenesis. The newly designed CHIKV mutants and their replicons open a wide range of possibilities for their application in different areas of research. First, they can be further developed as new vaccine candidates to additionally improve already attenuated strain CHIKV 181/25, which previously remained capable of inducing some adverse effects. The wt level of replication of these mutants suggests that during serial virus passage, it may be no selection pressure for their further evolution to more efficiently replicating phenotype. The replacement of three amino acids in nsP2 instead of making point mutations makes also an additional input into the stability of attenuated phenotype during virus production in the cells deficient in type I IFN induction and signaling. Second, similar mutants designed on the basis of wt CHIKV genome can be applied for studying critical aspects of CHIKV-host interactions and pathogenesis. Third, stable cell lines that contain noncytopathic CHIKV replicons can be used for screening of antiviral drugs without using replication-competent infectious CHIKV.

### Example 3: Lack of nsP2-Specific Nuclear Functions Attenuates Chikungunya Virus Replication Both In Vitro and In Vivo

Alphaviruses are a group of small, enveloped viruses, which are broadly distributed over all continents including Antarctic areas. Some of them, such as Eilat virus (EILV), replicate only in mosquitoes, but most of alphaviruses circulate between mosquito vectors and vertebrate hosts. Based on geographical distribution, alphaviruses are divided into the New World (NW) and the Old World (OW) alphaviruses. Many NW representatives, exemplified by Venezuelan (VEEV), eastern (EEEV) and western (WEEV) equine encephalitis viruses induce severe meningoencephalitis. Infections caused by natural isolates of VEEV, EEEV, or WEEV have been shown to result in high mortality rates in humans and neurological sequelae among survivors. The OW alphaviruses, such as Sindbis (SINV) and Semliki Forest (SFV) viruses, are generally less pathogenic, and in humans, the major symptoms of the induced diseases are fever, rash and arthritis. Within recent years, one of the representatives of the OW alphaviruses, chikungunya virus (CHIKV), has spread widely in both hemispheres. This spread has led to epidemics of polyarthritis characterized by severe joint pain that can continue for months. Moreover, it is also neuroinvasive for newborns and causes meningitis and cognitive disabilities. Despite the significant threat to public health, to date, CHIKV pathogenesis is insufficiently understood on molecular, cellular and systemic levels, and no licensed vaccines have been developed.

CHIKV genome (G RNA) is a ˜12 kb single stranded RNA of positive polarity. It mimics the structure of cellular mRNAs in that it is capped at the 5′ terminus and has a poly(A) tail at the 3′ terminus. This G RNA serves as a template for translation of a handful of viral nonstructural proteins, nsP1-to-4, which mediate RNA replication and synthesis of subgenomic RNA (SG RNA). The latter RNA functions as an mRNA for synthesis of viral structural proteins that ultimately form infectious virions.

Alphavirus nsP2 protein exhibits an exceptionally wide range of activities in viral replication: i) mediates processing of ns polyprotein precursors P123 and P1234, ii) functions as a helicase in viral RNA synthesis, iii) has NTPase activity and iv) serves as RNA phosphatase of viral G and SG RNAs in the cascade of capping reactions. Mutations in alphavirus nsP2-coding sequence may decrease and increase replication of viral RNAs. Additionally, while CHIKV nsP2 is an important structural and functional component of viral RCs, within infected vertebrate cells, a large fraction of this protein is distributed in the cytoplasm and nuclei suggesting additional functions in viral replication. As do nsP2 proteins of other OW alphaviruses, CHIKV nsP2 employs a mechanism similar to cellular transcription-coupled repair to rapidly degrade the main catalytic subunit (RPB1) of cellular DNA-dependent RNA polymerase II. During the OW alphavirus infections, degradation of RBP1 ultimately results in global inhibition of cellular transcription. It serves as a very efficient means of CHIKV interference with induction of cell signaling and activation of antiviral genes. Thus, CHIKV nsP2 is an important player in downregulation of the innate immune response, and this makes nsP2 an important target for modifications that may lead to the development of attenuated viral variants.

The accumulating data strongly suggest that the very carboxy-terminal S-adenosyl methionine-guanylyl transferase (SAM MTase)-like domain of nsP2 plays a critical role in the protein's transcription inhibition function(s). Point mutations in this domain can affect the ability of SINV- and SFV-specific G RNAs that lack structural genes (replicons) to induce transcriptional shutoff and cytopathic effect (CPE). Most of the identified mutations also strongly alter replication rates of viral genomes and transcription of SG RNAs. However, recently, we have identified a small, highly variable loop (VLoop) on the surface of SINV and CHIKV nsP2-specific SAM MTase-like domains that determine the nuclear functions of the protein. The designed CHIKV replicons and CHIK viruses encoding mutated VLoop lost the ability to downregulate cellular transcription in rodent cell lines and thus, became either less cytopathic or entirely noncytopathic. Most importantly, the introduced mutations did not have negative effects on viral replication rates in rodent cells that demonstrate intact I IFN induction and signaling. Thus, the introduced mutations affected the nuclear functions of CHIKV nsP2 without altering its activity in viral RCs.

In the present study, we continued to investigate the effects of the CHIKV nsP2 mutations on viral pathogenesis both in vitro and in vivo. Our data demonstrate that i) in contrast to parental CHIKV 181/25, the developed viral mutants are very potent type I IFN inducers in human cells, despite retaining efficient replication rates; ii) the mutations strongly attenuate a pathogenic variant of CHIKV in its ability to cause viremia in mice; however, iii) the designed mutants remain immunogenic. Thus, manipulations with CHIKV nsP2 SAM MTase-like domain may be used to improve safety of the previously designed attenuated CHIKV strain 181/25. The original strain has been shown to be highly immunogenic, but demonstrated some residual adverse effects in human trials, which need to be eliminated. Lastly, we show that additional mutations can be made within the macro domain of CHIKV nsP3, and they have a negative effect on viral cytopathogenicity in human cells. These mutations in nsP3 macro domain represent another means of CHIKV attenuation, and, if necessary, may be applied to improve safety of CHIKV vaccine candidates.

Cell cultures. The BHK-21 cells were kindly provided by Paul Olivo (Washington University, St. Louis, Mo.). The NIH 3T3, BJ-5ta, MRC-5, HFF-1, Vero clone 6 and HEK 293 cells were obtained from the American Tissue Culture Collection (Manassas, Va.). Huh7 cells were kindly provided by Charles Rice (Rockefeller University, New York, N.Y.). BHK-21, NIH 3T3, Vero and HEK 293 cells were maintained in alpha minimum essential medium supplemented with 10% fetal bovine serum (FBS) and vitamins. BJ-5ta, MRC-5, Huh7 and HFF-1 cells were maintained in Dulbecco's modified Eagle medium (DMEM) supplemented with 10% FBS.

Plasmid constructs. Plasmid encoding infectious cDNA of CHIKV 181/25 was provided by Dr. Scott Weaver (University of Texas Medical Branch at Galveston, Tex.). In the present study, this construct was used for making new modifications in nsP2- and nsP3-coding sequences. The infectious cDNA clone of more pathogenic CHIKV variant AF15561^{E2K200R:ΔUTR}, shown to be more pathogenic in mouse model of infection, was fully described elsewhere. The cDNA of this genome was reproduced on the basis of CHIKV 181/25 by PCR-based mutagenesis. The cDNAs of AF15561^{E2K200R:ΔUTR} and the designed mutants were cloned into a low-copy number plasmid under the control of CMV promoter. The poly(A) tail of the viral genome was also fused with the hepatitis delta ribozyme (RBZ) sequence. To simplify presentation, the AF15561^{E2K200R:ΔUTR} variant is referred in the text as wCHIKV. CHIKV/GFP that encodes GFP gene under control of subgenomic promoter has been described elsewhere. All the mutations introduced into nsP2 and nsP3 are indicated in the figures. They were designed using a PCR-based approach and other standard recombinant DNA techniques. RNA transcriptions, RNA infectivity assay, and rescue of recombinant viruses.

Plasmids were purified by ultracentrifugation in CsCl gradients. Viral G RNAs were synthesized in vitro by using SP6 RNA polymerase (Invitrogen) in the presence of cap analog (New England BioLabs) according to the recommendations of the manufacturers. The quality and yields of the transcripts were analyzed by agarose gel electrophoresis under nondenaturing conditions. Aliquots of the reaction mixtures were used without additional purification for electroporation of BHK-21 cells in the previously described conditions. Viruses were harvested at 24 h post electroporation, and titers were determined by plaque assay on BHK-21 cells.

RNA infectivities were analyzed in an infectious center assay (ICA). Briefly, the 10-fold dilutions of cells electroporated with the in vitro-synthesized RNAs were seeded in 6-well Costar plates with monolayers of naïve BHK-21 cells. After 2 h of incubation at 37° C., cells were covered by 0.5% agarose supplemented with DMEM and 3% FBS. Plaques were stained by crystal violet 3 days post electroporation. RNA infectivities were determined as PFU/μg of the in vitro-synthesized RNAs.

The parental wCHIKV (AF15561^{E2K200R:ΔUTR}) and its derivatives were rescued in BSL3 containment by transfecting plasmid DNA into BHK-21 cells using TransIT-X2 Transfection Reagent according to the manufacturer's recommendations (Mirus). Viruses were harvested at 48 h post transfection, and titers were determined by plaque assay on BHK-21 cells.

Analysis of viral RNA and protein synthesis. NIH 3T3 cells in 6-well Costar plates were infected with CHIKV 181/25 variants at a multiplicity of infection (MOI) 20 PFU/cell. Virus-specific RNAs were metabolically labeled between 4 and 8 h post infection (PI) in 0.8 ml of complete medium supplemented with [³H]uridine (20 mCi/ml) and Actinomycin D (1 mg/ml). RNAs were isolated from the cells by TRIzol reagent according to the manufacturer's recommendations (Invitrogen). RNAs were denatured by glyoxal and analyzed by agarose gel electrophoresis in sodium phosphate buffer. After impregnation with 2,5-diphenyoxazol (PPO), the gel was dried and used for autoradiography.

For protein labeling, cells in 6-well Costar plates were infected with CHIKV mutants at an MOI of 20 PFU/cell. At 7 h PI, they were washed with PBS, and proteins were metabolically labeled for 30 min at 37° C. in 0.8 ml of DMEM lacking methionine, and supplemented with [³⁵S]methionine (20 mCi/ml) and 0.1% FBS. Cells were harvested and lysed in the standard protein loading buffer for gel electrophoresis. Equal amounts of lysates were analyzed by gel electrophoresis in 10% NuPAGE gels (Invitrogen) followed by autoradiography.

RT-qPCR. Cells were infected with CHIKV variants indicated in the figure at an MOI of 20 PFU/cell. Cellular RNAs were isolated from 5×10⁵ cells using the Direct-zol RNA MiniPrep kit according to the manufacturer's instructions (Zymo Research). These RNA samples were used for cDNA synthesis by QuantiTect reverse transcription (RT) kit according to the manufacturer's instructions (Qiagen). The cDNAs were used for qPCR analysis with primers for the following mouse genes: IFN-β (NM_010510), IFIT1 (NM 008331), IFIT3 (NM_010501), ISG15 (NM_015783), and human genes: IFN-β (NM 002176), IFIT1 (NM_005101), IFIT3 (NM_001548), ISG15 (NM_001549). The qPCRs were performed using SsoFast EvaGreen supermix (Bio-Rad) in a CFX96 real-time PCR detection system (Bio-Rad). The specificities of the qPCR products were confirmed by analyzing their melting temperatures. The data were normalized to the mean threshold cycle (CT) of 18S ribosomal RNA in each sample. The fold difference was calculated using the ΔΔCT method.

Animal studies. To evaluate the ability of candidate viruses to cause viremia in mice, and to assess the resulting Ab response, 2-to-3-week old C57Bl/6 mice were inoculated into the left foot pad with 5×10³ PFU of indicated viruses diluted in PBS containing 1% mouse serum. All of the animal studies were carried out under the approval of the Institutional Animal Care and Use Committee of the University of Alabama at Birmingham (UAB). The experiments with CHIKV 181/25- and wCHIKV-based mutants were carried out in the ABSL2 and ABSL3 facilities, respectively. Animals were monitored daily for weight change, signs of disease or any abnormalities during the course of the experiment. At the times PI indicated in the figure legends, blood samples were taken from the retro-orbital sinus and sera were analyzed for either the levels of viremia or neutralizing Abs. All the mice were sacrificed humanely after the completion of the study.

Neutralizing antibody titers (PRNT₅₀). Serum samples were incubated at 50° C. for 1 h and then serially (2-fold) diluted in PBS supplemented with 1% FBS and 250 PFU/ml of CHIKV 181/25. Samples were incubated at 37° C. for 2 h, and 0.2 ml aliquots were applied to monolayers of BHK-21 cells in 6-well Costar plates. After 1 h-long incubation at 37° C., cells were overlaid with 0.5% agarose supplemented with DMEM and 3% FBS. After 3 days of incubation, plaques were stained with crystal violet. The percentage of reduction was plotted against the dilution to generate slope and intercept values using the best fit non-linear curves that were used to calculate the 50% reduction dilution (Graph Pad Prism software).

IFN-b induction. Cells were infected with CHIKV variants at MOIs indicated in the figure legends. Harvested samples were used to assess viral titers, and the levels of the mouse or human IFN-b were measured with a VeriKine Mouse or Human IFN-b ELISA kits, respectively, according to the manufacturer's recommendations (PBL Interferon Source).

CHIKV 181/25 variants with mutated VLoop are viable and less cytopathic. In our previous study, we selected a variety of CHIKV/GFP variants that contained mutations in the C-terminal SAM MTase-like domain of nsP2. These modifications altered the cytopathogenicity of the virus and made the designed mutants less cytopathic for rodent cells, but did not have negative effects on viral replication rates. These changes in viral phenotype resulted from replacement of VLoop (₆₇₄ATL₆₇₆ peptide) in CHIKV nsP2 by heterologous amino acid (aa) sequences. In this study, we further explored the effects of the mutations on viral attenuation, replication rates in vivo and in vitro and CHIKV immunogenicity.

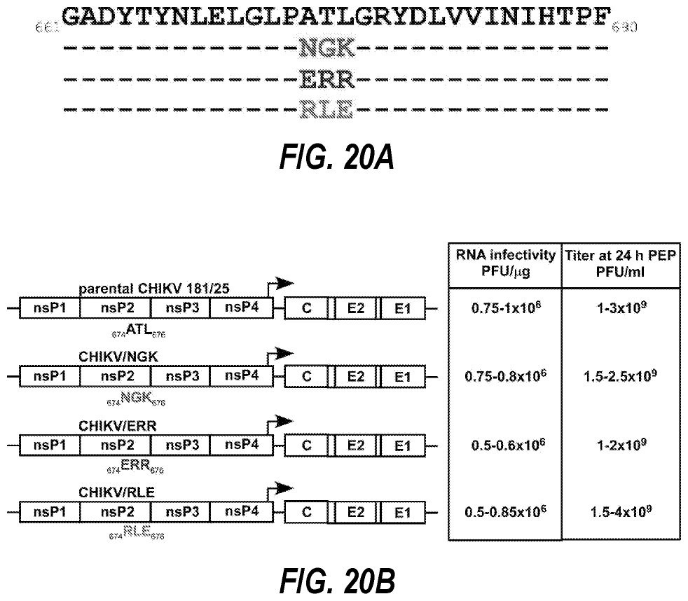

The genome of original CHIKV 181/25 was modified to contain the natural ₆₇₄ATL₆₇₆ peptide substituted by NGK, ERR, and RLE aa sequences (FIG. 20A). Unlike the previously used CHIKV/GFP, genomes of the newly designed mutants, termed CHIKV/NGK, CHIKV/ERR, and CHIKV/RLE, encoded no heterogenous genes. Infectivities of the in vitro-synthesized RNAs were evaluated in the ICA and viral stocks were harvested at 24 h post RNA transfection. In repeat experiments, the newly designed and parental CHIKV 181/25 constructs reproducibly demonstrated identical RNA infectivities (FIG. 20B). This precluded the possibility that acquisition of additional adaptive mutations was necessary for their viability. Similar infectious titers in the harvested stocks of CHIKV 181/25 and the mutants also suggested their efficient growth in BHK-21 cells (FIG. 20B). Notably, we observed a complete cytopathic effect (CPE) within 24 h post electroporation of RNA of parental CHIKV 181/25. However, despite efficient replication, the designed mutants did not induce CPE, and electroporated BHK-21 cells continued to grow. To our convenience, the mutants were still able to develop plaques in this cell line under agarose cover in the presence of low concentration of FBS. This strongly simplified the assessments of viral titers.

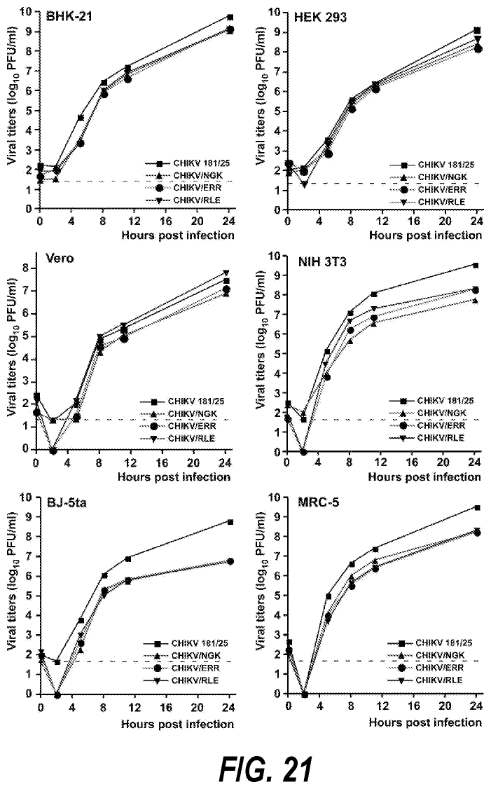

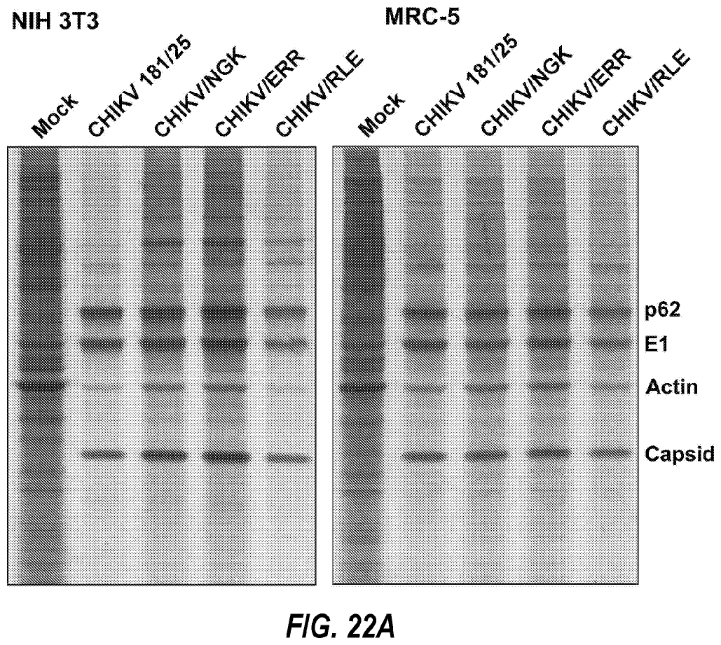

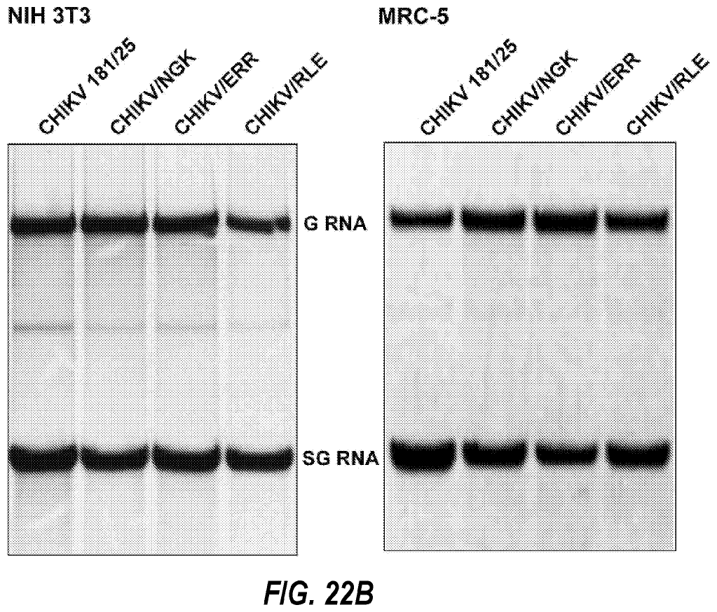

The designed CHIKV nsP2 mutants replicate in a variety of cell lines. Next, experiments were aimed at comparing replication rates of the designed mutants with those of parental CHIKV 181/25 in the cells of different origins (FIG. 21). Some of the used cell lines, such as human fibroblasts MRC-5, BJ-5ta and mouse NIH 3T3 fibroblasts, were fully competent in type I IFN induction and signaling. HEK 293 cells were used as less efficient type I IFN inducers, and others, BHK-21 and Vero cells, were applied as cell lines having defects in either type I IFN induction or signaling. All of the above cell lines were infected at an MOI of 0.01 PFU/cell, and virus release was assessed at different times post infection (PI). CHIKV nsP2 mutants demonstrated efficient replication in all cell types with the highest titers in BHK-21 and HEK 293 cells. However, in the cells competent in type I IFN induction and signaling (BJ-5ta, MRC-5 and NIH 3T3), at the late times PI, final titers of the mutants were reproducibly lower than those of the parental CHIKV 181/25. This decrease in viral replication was not a result of alterations in either G and SG RNA synthesis or translation of structural proteins. NsP2 mutants and parental CHIKV 181/25 exhibited similar efficiencies of RNA synthesis and translation of structural proteins (FIGS. 22A-22B). Thus, the plausible explanation for the detected lower titers of the mutants in the experiments performed at low MOI was that in contrast to CHIKV 181/25, they became efficient type I IFN inducers.

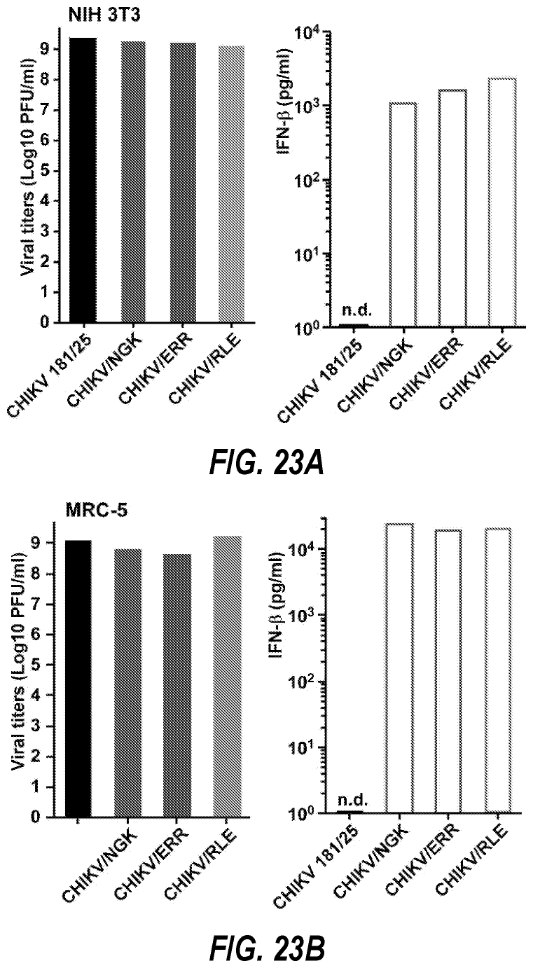

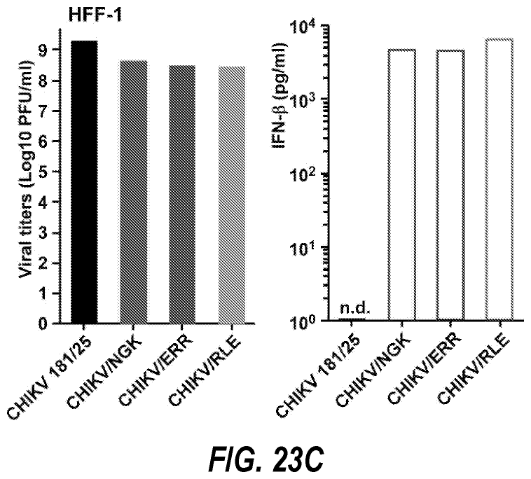

NsP2-specific mutations make CHIKV a potent IFN-b inducer. Next, we evaluated the abilities of CHIKV nsP2 mutants to induce type I IFN in mouse NIH 3T3 and human MRC-5 and HFF-1 fibroblasts (FIGS. 23A-23C). Cells were infected with parental CHIKV 181/25 and the designed mutants at high MOI (20 PFU/cell). Media were harvested at 18 h PI, and viral titers and concentrations of the released IFN-b were determined in the same samples. In all of the used cell lines, CHIKV 181/25 induced IFN-b very inefficiently, if at all (FIGS. 23A-23C), and its concentrations remained at the limit of detection. In repeat experiments, the designed mutants reproducibly replicated to essentially the same titers as the parental CHIKV 181/25 (see the left panels in FIGS. 23A-23C), but, in contrast, were very potent IFN-b inducers, particularly in MRC-5 cells. Taken together, these results demonstrated that mutations in VLoop altered the interferon-inhibiting effect of nsP2 protein in both mouse and human cells.

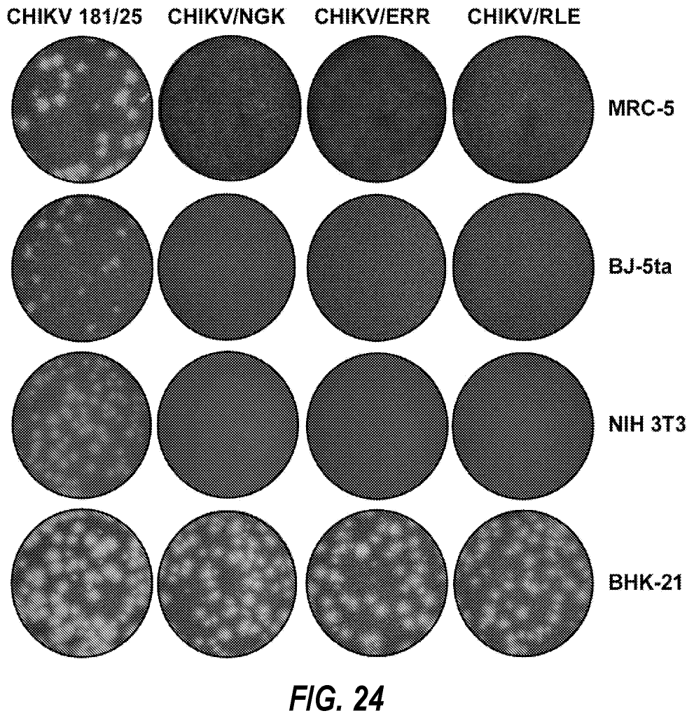

Consequently, the inability of the viruses to interfere with induction of IFN-b made them incapable of forming plaques in the cell lines having no defects in type I IFN induction and signaling (FIG. 24). On BHK-21 cells, the mutants and parental virus produced readily detectable plaques of similar sizes. However, in NIH 3T3, MRC-5 and BJ-5ta cells, plaques did not develop. The most likely explanation for this inability was that IFN-b released by primarily infected cells activated the antiviral state in the surrounding cells and protected them against subsequent rounds of infection.

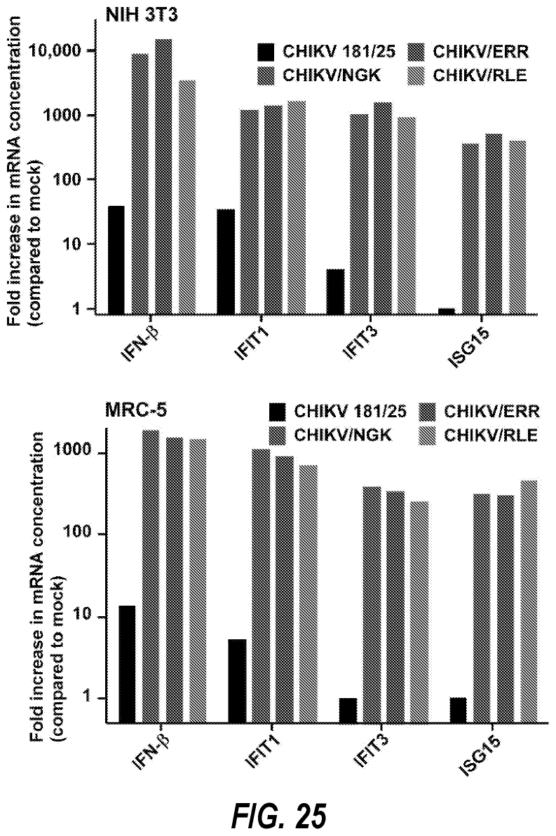

Replication of CHIKV nsP2 mutants results in activation of interferon stimulated genes (ISGs). In additional experiments, we tested whether cells infected with the mutants were not only releasing IFN-b, but also remained competent in IFN signaling and ultimate activation of ISGs. NIH 3T3 and MRC-5 cells were infected at an MOT of 20 PFU/cell and, at 16 h PI, the induction of selected ISGs and IFN-b was evaluated by RT-qPCR. Compared to parental CHIKV 181/25, cells infected with the mutants demonstrated a few orders of magnitude more efficient activation of ISGs (FIG. 25). Thus, the nsP2-specific mutations strongly altered the ability of CHIKV 181/25 to inhibit the antiviral response in both human and mouse fibroblasts.

CHIKV nsP2 mutants remain immunogenic in mice. Taken together, the accumulated data demonstrated that nsP2 VLoop-specific mutations affected CHIKV 181/25 infection spread in vitro. Infected cells remained capable of efficient type I IFN induction and responded by ISG activation. These new characteristics suggested that the introduced mutations could improve the currently available CHIKV 181/25 in terms of its attenuation level and safety.

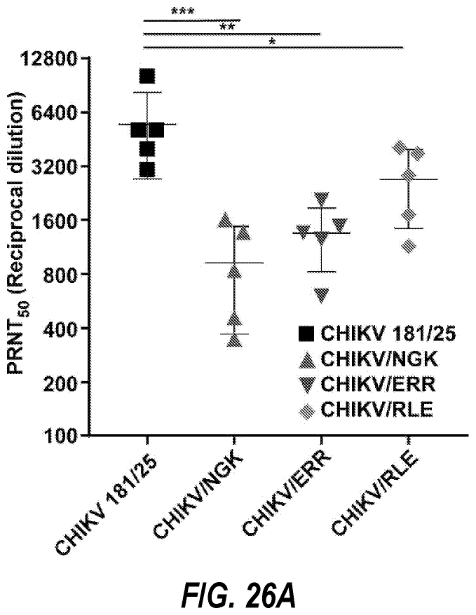

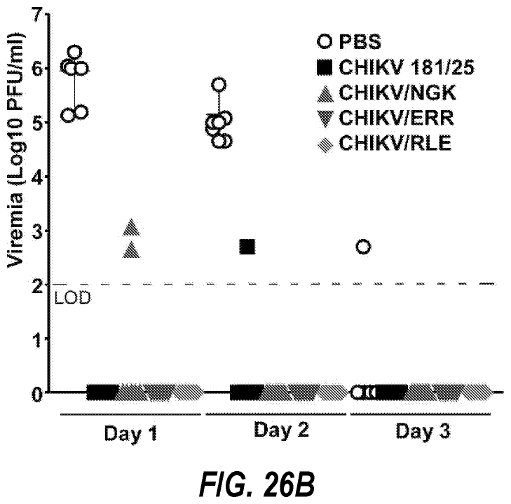

To understand the effect of the mutations on CHIKV replication in vivo, 2-to-3-week-old C57BL/6 mice were infected with the same doses of parental CHIKV 181/25 and nsP2 mutants in the left foot pad. The blood samples were collected on days 1, 2 and 3 PI and tested for levels of viremia (FIG. 26A). Only two samples taken on days 1 and 2 from CHIKV 181/25-infected mice showed presence of the virus (100 and 50 pfu/ml, respectively). In all other mice, the levels of viremia caused by either parental virus or the mutants were below the limit of detection. We monitored swelling of the joints or weight loss that are indications of morbidity, but in all groups, no significant changes were detected. Thus, these experiments did not generate direct, conclusive data about changes in replication levels of the mutant viruses in vivo.

To confirm replication of the mutants and immunogenicity if any, we collected blood samples on day 21 PI and tested for the levels of neutralizing antibodies. The results demonstrated that mutants remained competent in replication in vivo. They induced readily detectable levels of CHIKV-specific, neutralizing antibodies, albeit noticeably less efficiently than the parental CHIKV181/25 (FIG. 26A). At day 25 PI, the immunized mice were also challenged with more pathogenic variant of CHIKV, wCHIKV, to show the protective effect of vaccination. Blood samples were collected on day 1, 2, and 3 post challenge to assess the levels of viremia. In the unimmunized mice (PBS group), wCHIKV induced high levels of viremia that remained detectable on days 2 and 3 (FIG. 26B). Only two mice immunized with CHIKV/NGK demonstrated low viremia at day 1 post challenge, as well as did one mouse immunized with CHIKV 181/25 at day 2. No infectious virus was detected in mice immunized with CHIKV/ERR and CHIKV/RLE at any time post challenge. Thus, the designed CHIKV 181/25-based mutants remained immunogenic and offered protection from wCHIKV infection.

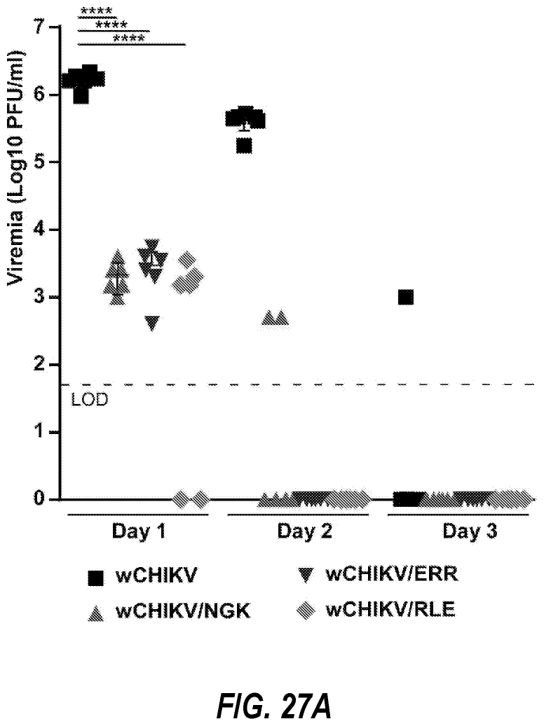

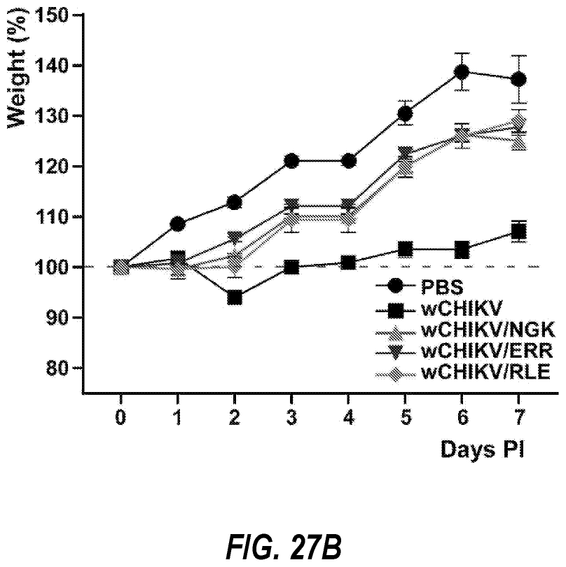

NsP2-specific mutations attenuate wCHIKV replication in vivo. In the above experiments, the mutations were introduced into nsP2 of CHIKV 181/25, which is an already attenuated variant of CHIKV. Indirect evidence, such as lower titers of neutralizing antibodies, indicated that the designed mutants became more attenuated. In order to generate better data about the effects of VLoop replacements on viral replication in vivo, we cloned the above-described mutant VLoop-encoding sequences into the genome of wCHIKV. The latter virus was used in challenge experiments described in the previous section. The designed variants were termed wCHIKV/NGK, wCHIKV/ERR and wCHIKV/RLE. These viruses and parental wCHIKV were rescued by transfecting the plasmids containing cDNAs of viral genomes under control of a CMV promoter. Two-to-three-week-old C57BL/6 mice were infected with same dose, 5×10³ PFU, of the rescued viruses, and we assessed the levels of induced viremia on days 1, 2 and 3 PI (FIG. 27A). On day 1, mice infected with parental wCHIKV exhibited viremia at the level of 10⁶ PFU/ml, and it continued on day 2 PI. At day 3, viremia above the level of detection was found in one mouse. Mice infected with wCHIKV also demonstrated the delay in weight gain (FIG. 27B). The designed nsP2 mutants exhibited almost 3 orders of magnitude lower viremia even at day 1 (FIG. 27A), and only 2 mice infected with wCHIKV/NGK remained positive for viremia at day 2. On days 2 and 3, in other samples, presence of the viruses was below the limit of detection. Mice infected with the mutants were gaining weight more efficiently than those in wCHIKV-infected group (FIG. 27B). Thus, the modifications in VLoop had strong negative effects on the ability of wCHIKV to develop viremia.

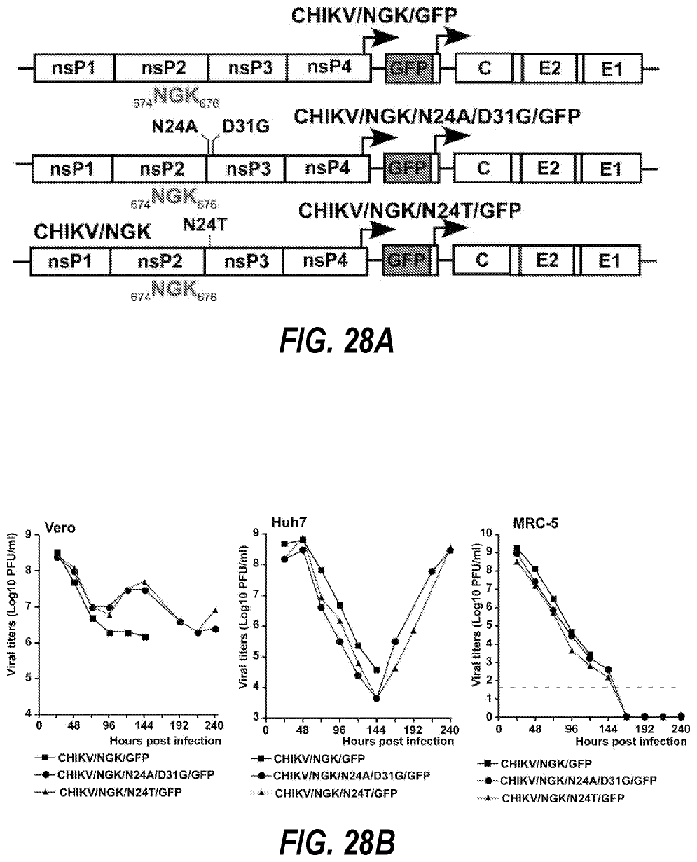

Additional CHIKV attenuation by mutations in the macro domain of nsP3. The nsP2 mutants, which were developed on the bases of either CHIKV 181/25 or wCHIKV, demonstrated dramatically lower cytopathogenicity in mouse cells and induced lower viremia in mice. However, mouse is not an ideal model to study human CHIKV-induced disease, and the designed recombinant viruses also retained cytopathogenicity in the tested cell lines of human origin. Therefore, we explored additional means of CHIKV attenuation by introducing mutations into nsP3 macro domain. As in our previous studies of the mechanism responsible for the SINV-induced cytopathic effect, the mutations, N24T and N24A/D32G, were aimed to inactivate the nsP3 macro domain-associated mono-ADP-ribosylhydrolase activity. They were introduced into genomes of both CHIKV/NGK/GFP (FIG. 28A) and wCHIKV/NGK, which already had the nsP2-specific VLoop replaced. In CHIKV 181/25-based constructs, GFP was left under control of the subgenomic promoter to better monitor viral replication in the absence of obvious CPE development.

The newly designed CHIKV 181/25-based nsP2/nsP3 mutants, CHIKV/NGK/N24A/D31G/GFP and CHIKV/NGK/N24T/GFP (FIG. 28A), exhibited remarkably reduced cytopathogenicity. Unlike the parental CHIKV/NGK/GFP, they could not form clear plaques in BHK-21 cells under agarose cover. These mutants were also able to persistently replicate in Vero and Huh7 cells, which are deficient in type I IFN response (FIG. 28B), while the parental CHIKV/GFP and CHIKV/NGK/GFP variants were causing complete CPE within 2-3 (data not shown) and 5-6 (FIG. 28B) days PI, respectively. Interestingly, the persistence of CHIKV/NGK/N24A/D31G/GFP and CHIKV/NGK/N24T/GFP in Vero cells developed in an unusual manner. In replicate experiments, a few days PI, Vero cells accelerated growth, and the mutants also demonstrated higher levels of replication. This was suggestive of viral adaptation; however, further investigation of this phenomenon was beyond the scope of this study. In contrast to Vero and Huh7 cells, the IFN competent human MRC-5 fibroblasts were able to stop and clear the established replication of nsP2/nsP3 mutants (FIG. 28B), while essentially all cell infected with parental CHIKV/NGK/GFP were non-viable by day 5 PI.

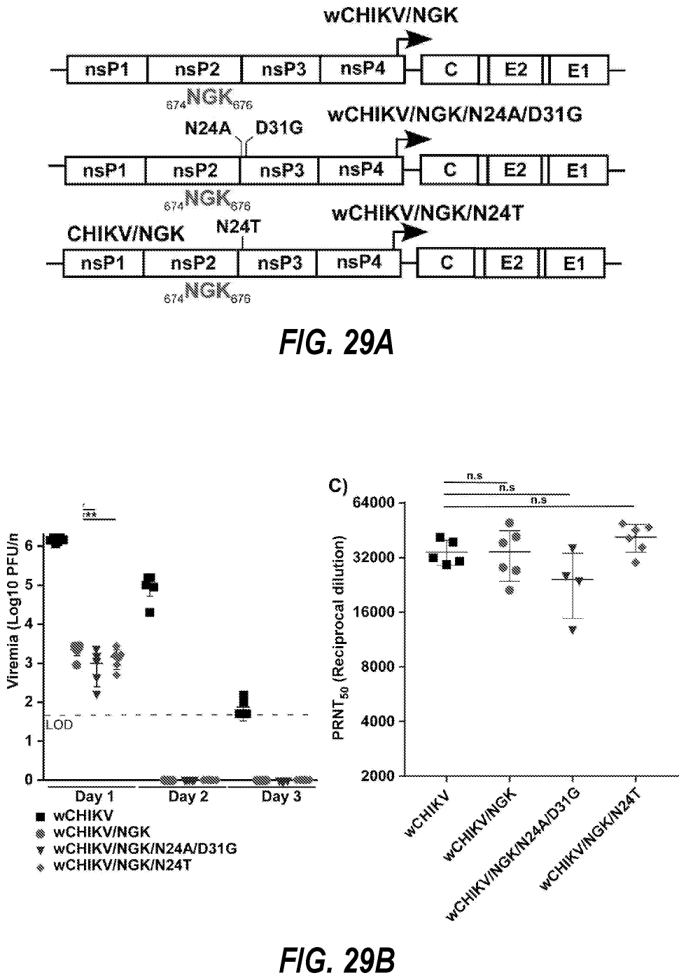

The described above nsP3 mutations were also introduced into the genome of wCHIKV/NGK (FIG. 29A). The designed wCHIKV/NGK/N24A/D31G and wCHIKV/NGK/N24T variants were rescued and used for in vivo study (FIG. 29B). Two-to-three-week-old C57BL/6 mice were infected with equal doses (5×10³ PFU) of nsP2/nsP3 mutants or parental wCHIKV and wCHIKV/NGK in the left foot pad. In this experiment (FIG. 29B), wCHIKV showed the highest level of viremia, which was ˜10⁶ PFU/ml on day 1, then decreased to ˜10⁵ PFU/ml at day 2, and dropped to the lowest level of detection on day 3. For all of the mutants, viremia on day 1 was more than 3 orders of magnitude lower and below the detection threshold on the subsequent days.

Despite 1000-fold lower serum viral levels, the nsP2 and nsP2/nsP3 mutants induced neutralizing Abs as efficiently as the parental wCHIKV (FIG. 29C). Even CHIKV/NGK/N24A/D31G and CHIKV/NGK/N24T mutants, which demonstrated additional attenuation in human cells, remained highly immunogenic. Taken together, these results supported the hypothesis that modifications in nsP2 and nsP3 can be used as alternative means to attenuate CHIKV. Such mutants demonstrate lower levels of replication in vivo, but remain immunogenic.

To date, live attenuated viral vaccines remain desirable due to their efficient induction of protective immunity against viral infections. The most common approach in the development of live attenuated vaccines is the serial passage of the wt viruses either in cultured cells or in chicken embryos. Such passaging of alphaviruses usually leads to accumulation of mutations in viral structural proteins and, in some cases, in the promoter of G RNA located in the 5′UTR. Mutations in E2 glycoprotein may make viral spikes capable of more efficient interaction with heparan sulfate at the plasma membrane. They increase alphavirus infectivity during propagation in cultured cells. Furthermore, the 5′UTR-specific mutations destabilize the RNA secondary structure and release the very 5′-terminal nucleotides from the stems, which are predicted for both 5′UTR of G RNA and the 3′end of the negative strand RNA intermediate. These structural changes improve the rates of G RNA replication and likely translation of nsPs. However, they also make evolved alphaviruses more sensitive to the antiviral effect of one of the ISG products, IFIT1, and thus, more attenuated. The passaging-based approach has previously been applied for the development of attenuated VEEV and CHIKV variants, TC-83 and 181/25 strains, respectively. CHIKV 181/25 was attenuated by serial passage of Asian strain 15561 eleven times in Vero cells followed by 18 passages on MRC-5 cells. The attenuated phenotypes of the selected CHIKV and VEEV mutants rely on only two point mutations, and both viruses remain capable of causing adverse effects in some vaccines. Thus, in the case of CHIKV and VEEV, application of passaging-based approach had likely reached its limit; however, the developed strains, VEEV TC-83 and CHIKV 181/25, remained insufficiently attenuated. Nevertheless, they did become stable upon propagation in tissue culture, demonstrated significant attenuation and may be used for further improvement of their safety.

Interference with the development of the innate immune response is a common characteristic of many viral taxonomic groups. Alphaviruses are not an exception and have developed the abilities to downregulate cellular response to their replication and to inhibit cell signaling that is aimed at the establishment of the antiviral state in yet uninfected cells. Some of the alphaviruses, including CHIKV, induce type I IFN very inefficiently, if at all. As do most of the positive sense RNA viruses, alphaviruses isolate their dsRNA intermediates into membrane spherules, and this likely complicates sensing of these pathogen-associated molecular patterns (PAMPs) by cytoplasmic receptors (pattern recognition receptors, PRRs), such as RIG-I, MDA5 and PKR. However, this isolation is likely incomplete, and some of the viral mutants that demonstrate no alterations in spherule formation become potent type I IFN inducers. Moreover, alphavirus RCs can utilize cellular mRNA as templates for dsRNA synthesis. These dsRNA molecules may be included into spherules less efficiently and be also detected by cellular PRRs.

Consequently, besides the membrane spherule formation, alphaviruses employ another powerful mechanism of interfering with the induction of an antiviral response. Geographically isolated viral species induce robust transcriptional shutoff in vertebrate, but not in mosquito cells, despite using very different means of achieving this goal. In the case of OW alphaviruses, including CHIKV, inhibition of transcription is mediated by the nonstructural protein nsP2. A large fraction of nsP2 accumulates in the nucleus, and within 4 h PI, presence of RPB1, the catalytic subunit of cellular DNA-dependent RNA polymerase II, drops to undetectable levels. Expression of CHIKV or SINV nsP2 proteins alone has deleterious effect on the overall cellular transcription and ultimately causes cell death. Thus, alterations of the nuclear functions of CHIKV nsP2 could make virus i) less cytopathic, ii) transform it into a potent type I IFN inducer, iii) attenuate viral infection in vivo and iv) improve the safety of already available, attenuated strain CHIKV 181/25. This possibility was supported by the results of our previous studies, in which we designed recombinant VEE/CHIKV variants encoding VEEV-specific nsPs and CHIKV structural proteins. The distinguishing characteristic of the latter viruses was that they expressed no viral proteins with transcription inhibitory functions and demonstrated very attenuated phenotypes in mice, but remained immunogenic. Similarly, in other studies, VEEV TC-83 and EEEV FL93 viruses were designed to have mutations in the nuclear localization signals of their capsid proteins. Those modifications made capsids incapable of forming tetrameric complexes with CRM1 and importin-a/b and blocking nuclear pores. Consequently, viral mutants were also no longer able to inhibit cellular transcription and became attenuated in vivo. Taken together, these data suggested that alphavirus-specific nuclear functions play critical roles in viral pathogenesis. Thus, CHIKV nsP2 can be exploited as a target for mutations aimed at viral attenuation. However, modifications of the latter protein to make it incapable of interfering with nuclear functions is a more delicate task than introducing mutations into the RNA-binding domain of VEEV or EEEV capsid proteins. Such point mutations are well tolerated by the highly variable, disordered, positively charged, RNA-binding domain of capsid protein. NsP2, in contrast, exhibits a variety of enzymatic functions in viral RNA synthesis. Their alteration by mutagenesis may either have deleterious effects on viral RNA replication or be lethal for the virus.

In the recent study, we have identified a small peptide on the surface of the C-terminal domain of the OW nsP2 (VLoop) that can be modified without affecting protease, helicase or other protein's functions. Here, the replacements of VLoop in CHIKV 181/25 by selected heterologous peptides affected only the nuclear function(s) of nsP2 without having any negative effects on viral replication in cells having defects in type I IFN induction or signaling (FIG. 21). In IFN-competent murine and human cells in particular, the designed CHIKV 181/25 variants, but not the parental CHIKV 181/25, were capable of inducing very high levels of IFN-b (FIGS. 23A-23C), which strongly affected viral spread. The infected cells also remained able to respond to IFN-b release by activating ISG (FIG. 25).

The lack of nsP2-specific nuclear function caused by the replacements of VLoop, decreased the ability of wCHIKV to develop viremia in mice by 3 orders of magnitude. In the background of CHIKV 181/25, these modifications of nsP2 also had a small, but detectable, negative effect on the levels of induced neutralizing Abs. This was an additional indirect indication that viral replication in vivo was affected. However, most importantly, CHIKV 181/25 mutants remained immunogenic, and the levels of neutralizing Abs were comparable to those induced by parental CHIKV 181/25 (FIG. 26A).

The same replacements of nsP2-specific VLoop in the more pathogenic wCHIKV variant also strongly attenuated viral replication in vivo (FIGS. 27A and 29B). On day 1 PI, the levels of viremia developed by wCHIKV/NKG, wCHICKV/ERR and wCHIKV/RLE were lower than that of parental wCHIKV. Thus, the results obtained on wCHIKV strongly correlate with the data from CHIKV 181/25-based experiments. Interestingly, despite replicating to 1,000-fold lower titers in vivo, the wCHIKV nsP2 mutants induced very high levels of neutralizing Abs. A plausible explanation for their efficiency in the induction of humoral immune response is that, similar to what was described for VEEV replicons, the induced by the mutants cytokines and, most importantly, IFN-b function as potent adjuvants that have a positive effect on the antibody response.

A distinguishing characteristic of alpha- and other RNA+ viruses is the high rate of their evolution. Viruses containing point mutations that affect replication are highly unstable. Their passaging in cultured cells leads to rapid generation and selection of more efficiently replicating variants, which usually accumulate either true reverting or second site mutations. The essentially wt levels of replication of these newly designed CHIKV nsP2 mutants in vitro and the replacements of the entire VLoop peptide instead of making point mutations suggested that reversion of the mutants to parental phenotype is a less probable event. Importantly, the original strain CHIKV 181/25 is already highly attenuated and so far, there is no indication that it can cause persistent arthritis. Therefore, in the unlikely case that the nsP2 mutations revert to the CHIKV 181/25-specific sequence, this will not result in the generation of pathogenic wild type CHIKV. The reversion of all of the nsP2- and E2-specific mutations at the same time to produce a natural strain of CHIKV is an even less likely event.

Since mice are not an adequate animal model for CHIK fever, the possibility that designed CHIKV 181/25 nsP2 mutants will be capable of inducing adverse effects in humans cannot be completely ruled out. Moreover, these mutants were noncytopathic in murine cells, but remained cytopathic in human cell lines. However, previously, we found that to become noncytopathic, SINV nsP2 mutants required additional modifications in the macro domain of nsP3, which altered virus-induced translational shutoff. In this study, similar nsP3-specific mutations also affected cytopathogenicity of CHIKV in human cells without deleterious effects on viral replication rates (FIGS. 28A-28B). They can be applied for additional attenuation of CHIKV. However, the proposed modifications in nsP2 will most likely be sufficient for the development of safer alternatives of CHIKV 181/25.

In conclusion, the results of this study demonstrate that i) CHIKV-specific nsP2 can be modified to make the virus a potent type I IFN inducer in mouse and human cells without affecting its in vitro replication rates. ii) The introduced mutations have a negative effect on the levels of viremia caused by CHIKV 181/25 and more pathogenic variant wCHIKV, but both variants remain capable of inducing neutralizing Abs. iii) If necessary, CHIKV can be additionally attenuated through the introduction of selected mutations into the macro domain of nsP3. Thus, CHIKV attenuation can be achieved through a rational design of mutations in its nonstructural genes. Such mutations affect viral inhibitory functions without deleterious effects on its replication. This rationale can improve attenuation, safety and stability of CHIKV variants attenuated by other approaches. Importantly, similar VLoop mutants can be rapidly developed for other pathogenic OW alphaviruses.

While there are shown and described particular embodiments of the invention, it is to be understood that the invention is not limited thereto but may be otherwise variously embodied and practiced within the scope of the following claims. Since numerous modifications and alternative embodiments of the present invention will be readily apparent to those skilled in the art, this description is to be construed as illustrative only and is for the purpose of teaching those skilled in the art the best mode for carrying out the present invention. Accordingly, all suitable modifications and equivalents may be considered to fall within the scope of the following claims.

All references cited herein, including non-patent publications, patent applications, GenBank® Database accession numbers and patents, are incorporated by reference herein in their entireties to the same extent as if each was individually and specifically indicated to be incorporated by reference, and was reproduced in its entirety herein.

Sequences

nsP2_SINV (NP_740671)
(SEQ ID NO: 3)
ALVETPRGHVRIIPQANDRMIGQYIVVSPNSVLKNAKLAPAHPLADQVKIITHSGRSG
RYAVEPYDAKVLMPAGGAVPWPEFLALSESATLVYNEREFVNRKLYHIAMHGPAK
NTEEEQYKVTKAELAETEYVEDVDKKRCVKKEEASGLVLSGELTNPPYHELALEGL
KTRPAVPYKVETIGVIGTPGSGKSAIIKSTVTARDLVTSGKKENCREIEADVLRLRGM
QITSKTVDSVMLNGCHKAVEVLYVDEAFACHAGALLALIAIVRPRKKVVLCGDPMQ
CGFFNMMQLKVHFNHPEKDICTKTFYKYISRRCTQPVTAIVSTLHYDGKMKTTNPCK
KNIEIDITGATKPKPGDIILTCFRGWVKQLQIDYPGHEVMTAAASQGLTRKGVYAVR
QKVNENPLYAITSEHVNVLLTRTEDRLVWKTLQGDPWIKQPTNIPKGNFQATIEDWE
AEHKGIIAAINSPTPRANPFSCKTNVCWAKALEPILATAGIVLTGCQWSELFPQFADD
KPHSAIYALDVICIKFFGMDLTSGLFSKQSIPLTYHPADSARPVAHWDNSPGTRKYGY
DHAIAAELSRRFPVFQLAGKGTQLDLQTGRTRVISAQHNLVPVNRNLPHALVPEYKE
KQPGPVKKFLNQFKHHSVLVVSEEKIEAPRKRIEWIAPIGIAGADKNYNLAFGEPPQA
RYDLVFINIGTKYRNHHFQQCEDHAATLKTLSRSALNCLNPGGTLVVKSYGYADRN
SEDVVTALARKFVRVSAARPDCVSSNTEMYLIFRQLDNSRTRQFTPHHLNCVISSVYE
GTRDGVGA
nsP2_AURV (NP_632023)
(SEQ ID NO: 4)
ALVETPRGKIKIIPQEGDVRIGSYTVISPAAVLRNQQLEPIHELAEQVKIITHGGRTGRY
SVEPYDAKVLLPTGCPMSWQHFAALSESATLVYNEREFLNRKLHHIATKGAAKNTE
EEQYKVCKAKDTDHEYVYDVDARKCVKREHAQGLVLVGELTNPPYHELAYEGLRT
RPAAPYHIETLGVIGTPGSGKSAIIKSTVTLKDLVTSGKKENCKEIENDVQKMRGMTI
ATRTVDSVLLNGWKKAVDVLYVDEAFACHAGTLMALIAIVKPRRKVVLCGDPKQW
PFFNLMQLKVNENNPERDLCTSTHYKYISRRCTQPVTAIVSTLHYDGKMRTTNPCKR
AIEIDVNGSTKPKKGDIVLTCFRGWVKQGQIDYPGPGGHDRAASQGLTRRGVYAVR
QKVNENPLYAEKSEHVNVLLTRTEDRIVWKTLQGDPWIKYLINVPKGNETATLEEW
QAEHEDIMKAINSTSTVSDPFASKVNTCWAKAIIPILRTAGIELTFEQWEDLFPQFRND
QPYSVMYALDVICTKMFGMDLSSGIFSRPEIPLTFHPADVGRVRAHWDNSPGGQKFG
YNKAVIPTCKKYPVYLRAGKGDQILPIYGRVSVPSARNNLVPLNRNLPHSLTASLQK
KEAAPLHKELNQLPGHSMLLVSKETCYCVSKRITWVAPLGVRGADHNHDLHEGFPP
LSRYDLVVVNMGQPYRFHHYQQCEEHAGLMRTLARSALNCLKPGGTLALKAYGFA
DSNSEDVVLSLARKFVRASAVRPSCTQFNTEMFFVFRQLDNDRERQFTQHHLNLAVS
NIFDNYKDGSGA
nsP2_MAYV (NP_579968)
(SEQ ID NO: 5)
GVVETPRNALKVTPQDRDTMVGSYLVLSPQTVLKSVKLQALHPLAESVKIITHKGRA
GRYQVDAYDGRVLLPTGAAIPVPDFQALSESATMVYNEREFINRKLYHIAVHGAAL
NTDEEGYEKVRAESTDAEYVYDVDRKQCVKREEAEGLVMIGDLINPPFHEFAYEGL
KRRPAAPYKTTVVGVFGVPGSGKSGIIKSLVTRGDLVASGKKENCQEIMLDVKRYRD
LDMTAKTVDSVLLNGVKQTVDVLYVDEAFACHAGTLLALIATVRPRKKVVLCGDP
KQCGFFNLMQLQVNENHNICTEVDHKSISRRCTLPITAIVSTLHYEGRMRTTNPYNKP
VIIDTTGQTKPNREDIVLTCFRGWVKQLQLDYRGHEVMTAAASQGLTRKGVYAVR
MKVNENPLYAQSSEHVNVLLTRTEGRLVWKTLSGDPWIKTLSNIPKGNFTATLEDW
QREHDTIMRAITQEAAPLDVFQNKAKVCWAKCLVPVLETAGIKLSATDWSAIILAFK
EDRAYSPEVALNEICTKIYGVDLDSGLFSAPRVSLHYTTNHWDNSPGGRMYGFSVEA
ANRLEQQHPFYRGRWASGQVLVAERKTQPIDVTCNLIPFNRRLPHTLVTEYHPIKGE
RVEWLVNKIPGYHVLLVSEYNLILPRRKVTWIAPPTVTGADLTYDLDLGLPPNAGRY
DLVFVNMHTPYRLHHYQQCVDHAMKLQMLGGDALYLLKPGGSLLLSTYAYADRTS
EAVVTALARRFSSFRAVTVRCVTSNTEVFLLFTNFDNGRRTVTLHQTNGKLSSIYAG
TVLQAAGC
nsP2_RRV (NP_062879)
(SEQ ID NO: 6)
GVVETPRNALKVTPQERDQLIGAYLILSPQTVLKSEKLTPIHPLAEQVTIMTHSGRSGR
YPVDRYDGRVLVPTGAAIPVSEFQALSESATMVYNEREFINRKLHHIALYGPALNTD
EENYEKVRAERAEAEYVFDVDKRTCVKREDASGLVLVGDLINPPFHEFAYEGLKIRP
ATPFQTTVIGVFGVPGSGKSAIIKSVVTTRDLVASGKKENCQEIVNDVKKQRGLDVT
ARTVDSILLNGCRRGVENLYVDEAFACHSGTLLALIAMVKPTGKVILCGDPKQCGFF
NLMQLKVNFNHDICTQVLHKSISRRCTLPITAIVSTLHYQGKMRTTNLCSAPIQIDTTG
TTKPAKGDIVLTCFRXWVKQLQIDYRGHEVMTAAASQGLTRKGVYAVRQKVNENP
LYAPSSEHVNVLLTRTENRLVWKTLSGDPWIKVLTNIPKGDFSATLEEWQEEHDNIM
NALRERSTAVDPFQNKAKVCWAKCLVQVLETAGIRMTAEEWDTVLAFREDRAYSP
EVALNEICTKYYGVDLDSGLFSAQSVSLYYENNHWDNRPGGRMYGFNREVARKFE
QRYPFLRGKMDSGLQVNVPERKVQPFNAECNILLLNRRLPHALVTSYQQCRGERVE
WLLKKLPGYHLLLVSEYNLALPHKRVFWIAPPHVSGADRIYDLDLGLPLNAGRYDL
VFVNIHTEYRTHHYQQCVDHSMKLQMLGGDSLHLLXPGGSLLIRAYGYADRVSEM
VVTALARKFSAFRVLRPACVTSNTEVFLLFTNFDNGRRAVTLHQANQRLSSMFACN
GLHTAGC
nsP2_SFV (P08411) (SEQ ID NO: 7)
GVVETPRSALKVTAQPNDVLLGNYVVLSPQTVLKSSKLAPVHPLAEQVKIITHNGRA
GRYQVDGYDGRVLLPCGSAIPVPEFQALSESATMVYNEREFVNRKLYHIAVHGPSLN
TDEENYEKVRAERTDAEYVFDVDKKCCVKREEASGLVLVGELTNPPFHEFAYEGLKI
RPSAPYKTTVVGVFGVPGSGKSAIIKSLVTKHDLVTSGKKENCQEIVNDVKKHRGLD
IQAKTVDSILLNGCRRAVDILYVDEAFACHSGTLLALIALVKPRSKVVLCGDPKQCGF
FNMMQLKVNFNHNICTEVCHKSISRRCTRPVTAIVSTLHYGGKMRTTNPCNKPIIIDT
TGQTKPKPGDIVLTCFRGWVKQLQLDYRGHEVMTAAASQGLTRKGVYAVRQKVNE
NPLYAPASEHVNVLLTRTEDRLVWKTLAGDPWIKVLSNIPQGNFTATLEEWQEEHD
KIMKVIEGPAAPVDAFQNKANVCWAKSLVPVLDTAGIRLTAEEWSTIITAFKEDRAY
SPVVALNEICTKYYGVDLDSGLFSAPKVSLYYENNHWDNRPGGRMYGFNAATAAR
LEARHTFLKGQWHTGKQAVIAERKIQPLSVLDNVIPINRRLPHALVAEYKTVKGSRV
EWLVNKVRGYHVLLVSEYNLALPRRRVTWLSPLNVTGADRCYDLSLGLPADAGRF
DLVFVNIHTEFRIHHYQQCVDHAMKLQMLGGDALRLLKPGGSLLMRAYGYADKISE
AVVSSLSRKFSSARVLRPDCVTSNTEVFLLFSNFDNGKRPSTLHQMNTKLSAVYAGE
AMHTAGC
nsP2_GETV (Q5Y389) (SEQ ID NO: 8)
GVVETPRNALKVTPQAHDHLIGSYLILSPQTVLKSEKLAPIHPLAEQVTVMTHSGRSG
RYPVDKYDGRVLIPTGAAIPVSEFQALSESATMVYNEREFINRKLHHIALYGPALNTD
EESYEKVRAERAETEYVFDVDKKACIKKEEASGLVLTGDLINPPFHEFAYEGLKIRPA
APYHTTIIGVFGVPGSGKSAIIKNMVTTRDLVASGKKENCQEIMNDVKRQRGLDVTA
RTVDSILLNGCKKGVENLYVDEAFACHSGTLLALIALVRPSGKVVLCGDPKQCGFFN
LMQLKVHYNHNICTRVLHKSISRRCTLPVTAIVSTLHYQGKMRTTNRCNTPIQIDTTG
SSKPASGDIVLTCFRGWVKQLQIDYRGHEVMTAAASQGLTRKGVYAVRQKVNENPL
YSPLSEHVNVLLTRTENRLVWKTLSGDPWIKVLTNVPRGDFSATLEEWHEEHDGIM
RVLNERPAEVDPFQNKAKVCWAKCLVQVLETAGIRMTADEWNTILAFREDRAYSPE
VALNEICTRYYGVDLDSGLFSAQSVSLFYENNHWDNRPGGRMYGFNHEVARKYAA
RFPFLRGNMNSGLQLNVPERKLQPFSAECNIVPSNRRLPHALVTSYQQCRGERVEWL
LKKIPGHQMLLVSEYNLVIPHKRVFWIAPPRVSGADRTYDLDLGLPMDAGRYDLVF
VNIHTEYRQHHYQQCVDHSMRLQMLGGDSLHLLRPGGSLLMRAYGYADRVSEMV
VTALARKFSAFRVLRPACVTSNTEVFLLFSNFDNGRRAVTLHQANQKLSSMYACNG
LHTAGC
nsP2_CHIKV (ABO38822)
(SEQ ID NO: 9)
GIIETPRGAIKVTAQLTDHVVGEYLVLSPQTVLRSQKLSLIHALAEQVKTCTHSGRAG
RYAVEAYDGRVLVPSGYAISPEDFQSLSESATMVYNEREFVNRKLHHIAMHGPALNT
DEESYELVRAERTEHEYVYDVDQRRCCKKEEAAGLVLVGDLTNPPYHEFAYEGLKI
RPACPYKIAVIGVFGVPGSGKSAIIKNLVTRQDLVTSGKKENCQEISTDVMRQRGLEIS
ARTVDSLLLNGCNRPVDVLYVDEAFACHSGTLLALIALVRPRQKVVLCGDPKQCGE
FNMMQMKVNYNHNICTQVYHKSISRRCTLPVTAIVSSLHYEGKMRTTNEYNMPIVV
DTTGSTKPDPGDLVLTCFRGWVKQLQIDYRGHEVMTAAASQGLTRKGVYAVRQKV
NENPLYASTSEHVNVLLTRTEGKLVWKTLSGDPWIKTLQNPPKGNFKATIKEWEVE
HASMAGICSHQVTFDTFQNKANVCWAKSLVPILETAGIKLNDRQWSQIIQAFKEDK
AYSPEVALNEICTRMYGVDLDSGLFSKPLVSVYYADNHWDNRPGGKMFGFNPEAAS
ILERKYPFTKGKWNINKQICVTTRRIEDFNPTTNIIPVNRRLPHSLVAEHRPVKGERME
WLVNKINGHHVLLVSGYNLALPTKRVTWVAPLGVRGADYTYNLELGLPATLGRYD
LVVINIHTPFRIHHYQQCVDHAMKLQMLGGDSLRLLKPGGSLLIRAYGYADRTSERV
ICVLGRKFRSSRALKPPCVTSNTEMFFLFSNFDNGRRNFTTHVMNNQLNAAFVGQAT
RAGC
nsP2_ONNV (NP_041254)
(SEQ ID NO: 10)
GIVETPRGAIKVTAQPSDLVVGEYLVLTPQAVLRSQKLSLIHALAEQVKTCTHSGRA
GRYAVEAYDGRVLVPSGYAIPQEDFQSLSESATMVFNEREFVNRKLHHIAMHGPAL
NTDEESYELVRVEKTEHEYVYDVDQKKCCKREEATGLVLVGDLTSPPYHEFAYEGL
KIRPACPYKTAVIGVFGVPGSGKSAIIKNLVTRQDLVTSGKKENCQEISNDVMRQRKL
EISARTVDSLLLNGCNKPVEVLYVDEAFACHSGTLLALIAMVRPRQKVVLCGDPKQC
GFFNMMQMKVNYNHNICTQVYHKSISRRCTLPVTAIVSSLHYESKMRTTNEYNQPIV
VDTTGITKPEPGDLVLTCFRGWVKQLQIDYRGNEVMTAAASQGLTRKGVYAVRQK
VNENPLYAPTSEHVNVLLTRTEGKLTWKTLSGDPWIKILQNPPKGDFKATIKEWEAE
HASIMAGICNHQMAFDTFQNKANVCWAKCLVPILDTAGIKLSDRQWSQIVQAFKED
RAYSPEVALNEICTRIYGVDLDSGLFSKPLISVYYADNHWDNRPGGKMFGFNPEVAL
MLEKKYPFTKGKWNINKQICITTRKVDEFNPETNIIPANRRLPHSLVAEHHSVRGERM
EWLVNKISGHHMLLVSGHNLILPTKRVTWVAPLGTRGADYTYNLELGLPATLGRYD
LVVINIHTPFRIHHYQQCVDHAMKLQMLGGDSLRLLKPGGSLLIRAYGYADRTSERV
ISVLGRKFRSSRALKPQCITSNTEMFFLFSRFDNGRRNFTTHVMNNQLNAVYAGLAT
RAGC

1. An alphavirus nsP2 protein comprising one or more amino acid substitutions that disrupts the ability of nsP2 to induce RPB1 degradation and inhibition of cellular transcription, comprising at least a substitution at:
a) amino acid 674 in chikungunya virus (CHIKV);
b) amino acid 675 in CHIKV;
c) amino acid 676 in CHIKV; and/or
d) amino acid 677 in CHIKV,
or at the corresponding amino acid positions in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses.

2. The alphavirus nsP2 protein of claim 1, wherein the alphavirus is CHIKV and amino acid residues A674, 1675 and L677 are substituted with an amino acid other than wild type, such as wherein the substitutions at 674ATL676 are ERR, FFR, RSR, NGK, DID, RLH, MLR, VRR, SGV, RLE, RVP, KLN, OMS, HIK, FIH, LFD, EMS, IKW or YMS.

3. (canceled)

4. The alphavirus nsP2 protein of claim 1, wherein the alphavirus is SINV and amino acid residues P683 and/or Q684 are substituted with an amino acid other than wild type, such as wherein the substitutions are P6830, P683E, P683N, P683S and/or Q684P.

5. (canceled)

6. The alphavirus nsP2 protein of claim 1, where the alphavirus is SFV and amino acid residues A674, D675, A676 and/or G677 are substituted with an amino acid other than wild type, such as wherein the substitutions at 674ADA676 are NGK or RTE.

7. (canceled)

8. An attenuated alphavirus particle comprising a nucleic acid molecule encoding the alphavirus nsP2 protein of claim 1.

9. An immunogenic composition comprising the attenuated alphavirus particle of claim 8 in a pharmaceutically acceptable carrier.

10. A recombinant replicon nucleic acid, comprising:
a) the nucleotide sequence of a 5′ terminus of alphavirus genome that is required for genome translation and replication;
b) a nucleotide sequence encoding alphavirus nonstructural proteins nsP1, nsP3, nsP4 and nsP2, wherein said nsP2 comprises one or more amino acid substitutions, comprising at least substitutions at:
a) amino acid 674 in chikungunya virus (CHIKV);
b) amino acid 675 in CHIKV;
c) amino acid 676 in CHIKV; and/or
d) amino acid 677 in CHIKV,
or at the corresponding amino acid positions in Sindbis virus (SINV), Aura virus (AURV), Mayaro virus (MAYV), Ross River virus (RRV), Semliki Forest virus (SFV), Getah virus (GETV), or O' Nyong Nyong virus (ONNV);
c) at least one alphavirus subgenomic promoter;
d) at least one heterologous nucleic acid molecule; and
e) a nucleotide sequence encoding a 3′ terminus of alphavirus genome that functions in regulation of viral genome replication.

11-17. (canceled)

18. A vector comprising the recombinant replicon nucleic acid of claim 10.

19. A cell comprising the vector of 18.

20. A packaging cell (or producer cell) comprising the recombinant replicon nucleic acid of claim 10.

21-23. (canceled)

24. A method of making infectious alphavirus particles, comprising introducing the recombinant replicon nucleic acid of claim 10 into a helper cell under conditions whereby infectious alphavirus particles are produced in the helper cell.

25. (canceled)

26. An infectious alphavirus particle comprising the recombinant replicon nucleic acid of claim 10.

27. A composition comprising a population of infectious alphavirus replicon particles, wherein said particle contains the recombinant replicon nucleic acid of claim 10.

28. A composition comprising a population of attenuated alphavirus particles of claim 8.

29. A composition comprising the alphavirus nsP2 protein of claim 1, in a pharmaceutically acceptable carrier.

30. A method of delivering a nucleic acid to a cell, comprising introducing into the cell the recombinant replicon nucleic acid of claim 10.

31. A method of delivering a therapeutic heterologous protein and/or functional RNA to a subject, comprising administering to the subject the recombinant replicon nucleic acid of claim 10, wherein the replicon nucleic acid encodes a therapeutic heterologous protein and/or functional RNA, thereby delivering a therapeutic heterologous protein and/or functional RNA to the subject.

32. A method of producing a protein of interest in a cell, comprising introducing into the cell the recombinant replicon nucleic acid of claim 10, wherein the recombinant replicon nucleic acid comprises a nucleotide sequence encoding the protein of interest, under conditions whereby the recombinant replicon nucleic acid is expressed and the protein of interest is produced.

33. (canceled)

34. A method of inducing and/or enhancing an immune response in a subject, comprising administering to the subject an effective amount of the attenuated alphavirus particle of claim 8, thereby inducing and/or enhancing an immune response in the subject as compared with a control subject.

35. A method of treating and/or preventing an alphavirus infection and/or treating the effects of an alphavirus infection in a subject, comprising administering to the subject an immunogenic amount of the attenuated alphavirus particle of claim 8, thereby treating and/or preventing an alphavirus infection in the subject and/or treating the effects of an alphavirus infection in the subject.

36. (canceled)

37. A method of screening a test agent and/or compound for anti-alphavirus activity, comprising:
a) generating a cell line in which the recombinant replicon nucleic acid of this invention, encoding a marker protein such as green fluorescent protein (GFP) or luciferase, is persistently replicated;
b) introducing into cells of this cell line a test agent and/or compound; and
c) observing the effect of the presence of the test agent and/or compound on expression of the marker protein in the cell to evaluate the effect of the test agent and/or compound on the ability of the recombinant replicon nucleic acid to replicate, thereby identifying a test agent or compound that inhibits (evidenced by decreased marker signal) or enhances (evidenced by increased marker signal) recombinant replicon nucleic acid replication.

38. A method of attenuating an alphavirus, comprising substituting one or more than one amino acid residue in the variable (V) region of the nonstructural protein 2 (nsP2) of the alphavirus, wherein the one or more amino acid residues that are substituted are amino acids 674, 675, 677 and/or 678 of CHIKV or the corresponding amino acid residues in Sindbis virus (SINV, amino acid residues 683, 684 and/or 685), Aura virus (AURV, amino acid residues 682, 683 and/or 684), Mayaro virus (MAYV, amino acid residues 673, 674, 675 and/or 676), Ross River virus (RRV, amino acid residues 673, 674, 675 and/or 676), Semliki Forest virus (SFV, amino acid residues 674, 675, 676 and/or 677), Getah virus (GETV, amino acid residues 673, 674, 675 and/or 676), O' Nyong Nyong virus (ONNV, amino acid residues 674, 675, 676 and/or 677), or any new emerging Old World alphaviruses.

39. (canceled)

40. A vaccine formulation comprising the attenuated alphavirus particle of claim 8 in a vaccine diluent.


SEQUENCE LISTING (28 sequences)

SEQ ID NO: 1 - DNA, 20 bp, Artificial
  guide nucleic acid
gggaaccgggacacactctg

SEQ ID NO: 2 - DNA, 20 bp, Artificial
  guide nucleic acid
cagagtgtgtcccggttccc

SEQ ID NO: 3 - protein, 807 aa, Sindbis virus
ALVETPRGHVRIIPQANDRMIGQYIVVSPNSVLKNAKLAPAHPLADQVKIITHSGRSGRYAVEPYDAKVLMPAGGAVPWPEFLALSESATLVYNEREFVNRKLYHIAMHGPAKNTEEEQYKVTKAELAETEYVFDVDKKRCVKKEEASGLVLSGELTNPPYHELALEGLKTRPAVPYKVETIGVIGTPGSGKSAIIKSTVTARDLVTSGKKENCREIEADVLRLRGMQITSKTVDSVMLNGCHKAVEVLYVDEAFACHAGALLALIAIVRPRKKVVLCGDPMQCGFFNMMQLKVHFNHPEKDICTKTFYKYISRRCTQPVTAIVSTLHYDGKMKTTNPCKKNIEIDITGATKPKPGDIILTCFRGWVKQLQIDYPGHEVMTAAASQGLTRKGVYAVRQKVNENPLYAITSEHVNVLLTRTEDRLVWKTLQGDPWIKQPTNIPKGNFQATIEDWEAEHKGIIAAINSPTPRANPFSCKTNVCWAKALEPILATAGIVLTGCQWSELFPQFADDKPHSAIYALDVICIKFFGMDLTSGLFSKQSIPLTYHPADSARPVAHWDNSPGTRKYGYDHAIAAELSRRFPVFQLAGKGTQLDLQTGRTRVISAQHNLVPVNRNLPHALVPEYKEKQPGPVKKFLNQFKHHSVLVVSEEKIEAPRKRIEWIAPIGIAGADKNYNLAFGFPPQARYDLVFINIGTKYRNHHFQQCEDHAATLKTLSRSALNCLNPGGTLVVKSYGYADRNSEDVVTALARKFVRVSAARPDCVSSNTEMYLIFRQLDNSRTRQFTPHHLNCVISSVYEGTRDGVGA

SEQ ID NO: 4 - protein, 806 aa, Aura virus
ALVETPRGKIKIIPQEGDVRIGSYTVISPAAVLRNQQLEPIHELAEQVKIITHGGRTGRYSVEPYDAKVLLPTGCPMSWQHFAALSESATLVYNEREFLNRKLHHIATKGAAKNTEEEQYKVCKAKDTDHEYVYDVDARKCVKREHAQGLVLVGELTNPPYHELAYEGLRTRPAAPYHIETLGVIGTPGSGKSAIIKSTVTLKDLVTSGKKENCKEIENDVQKMRGMTIATRTVDSVLLNGWKKAVDVLYVDEAFACHAGTLMALIAIVKPRRKVVLCGDPKQWPFFNLMQLKVNFNNPERDLCTSTHYKYISRRCTQPVTAIVSTLHYDGKMRTTNPCKRAIEIDVNGSTKPKKGDIVLTCFRGWVKQGQIDYPGPGGHDRAASQGLTRRGVYAVRQKVNENPLYAEKSEHVNVLLTRTEDRIVWKTLQGDPWIKYLTNVPKGNFTATLEEWQAEHEDIMKAINSTSTVSDPFASKVNTCWAKAIIPILRTAGIELTFEQWEDLFPQFRNDQPYSVMYALDVICTKMFGMDLSSGIFSRPEIPLTFHPADVGRVRAHWDNSPGGQKFGYNKAVIPTCKKYPVYLRAGKGDQILPIYGRVSVPSARNNLVPLNRNLPHSLTASLQKKEAAPLHKFLNQLPGHSMLLVSKETCYCVSKRITWVAPLGVRGADHNHDLHFGFPPLSRYDLVVVNMGQPYRFHHYQQCEEHAGLMRTLARSALNCLKPGGTLALKAYGFADSNSEDVVLSLARKFVRASAVRPSCTQFNTEMFFVFRQLDNDRERQFTQHHLNLAVSNIFDNYKDGSGA

SEQ ID NO: 5 - protein, 798 aa, Mayaro virus
GVVETPRNALKVTPQDRDTMVGSYLVLSPQTVLKSVKLQALHPLAESVKIITHKGRAGRYQVDAYDGRVLLPTGAAIPVPDFQALSESATMVYNEREFINRKLYHIAVHGAALNTDEEGYEKVRAESTDAEYVYDVDRKQCVKREEAEGLVMIGDLINPPFHEFAYEGLKRRPAAPYKTTVVGVFGVPGSGKSGIIKSLVTRGDLVASGKKENCQEIMLDVKRYRDLDMTAKTVDSVLLNGVKQTVDVLYVDEAFACHAGTLLALIATVRPRKKVVLCGDPKQCGFFNLMQLQVNFNHNICTEVDHKSISRRCTLPITAIVSTLHYEGRMRTTNPYNKPVIIDTTGQTKPNREDIVLTCFRGWVKQLQLDYRGHEVMTAAASQGLTRKGVYAVRMKVNENPLYAQSSEHVNVLLTRTEGRLVWKTLSGDPWIKTLSNIPKGNFTATLEDWQREHDTIMRAITQEAAPLDVFQNKAKVCWAKCLVPVLETAGIKLSATDWSAIILAFKEDRAYSPEVALNEICTKIYGVDLDSGLFSAPRVSLHYTTNHWDNSPGGRMYGFSVEAANRLEQQHPFYRGRWASGQVLVAERKTQPIDVTCNLIPFNRRLPHTLVTEYHPIKGERVEWLVNKIPGYHVLLVSEYNLILPRRKVTWIAPPTVTGADLTYDLDLGLPPNAGRYDLVFVNMHTPYRLHHYQQCVDHAMKLQMLGGDALYLLKPGGSLLLSTYAYADRTSEAVVTALARRFSSFRAVTVRCVTSNTEVFLLFTNFDNGRRTVTLHQTNGKLSSIYAGTVLQAAGC

SEQ ID NO: 6 - protein, 798 aa, Ross River virus
  misc_feature (362)..(362): Xaa can be any naturally occurring amino acid
  misc_feature (716)..(716): Xaa can be any naturally occurring amino acid
GVVETPRNALKVTPQERDQLIGAYLILSPQTVLKSEKLTPIHPLAEQVTIMTHSGRSGRYPVDRYDGRVLVPTGAAIPVSEFQALSESATMVYNEREFINRKLHHIALYGPALNTDEENYEKVRAERAEAEYVFDVDKRTCVKREDASGLVLVGDLINPPFHEFAYEGLKIRPATPFQTTVIGVFGVPGSGKSAIIKSVVTTRDLVASGKKENCQEIVNDVKKQRGLDVTARTVDSILLNGCRRGVENLYVDEAFACHSGTLLALIAMVKPTGKVILCGDPKQCGFFNLMQLKVNFNHDICTQVLHKSISRRCTLPITAIVSTLHYQGKMRTTNLCSAPIQIDTTGTTKPAKGDIVLTCFRXWVKQLQIDYRGHEVMTAAASQGLTRKGVYAVRQKVNENPLYAPSSEHVNVLLTRTENRLVWKTLSGDPWIKVLTNIPKGDFSATLEEWQEEHDNIMNALRERSTAVDPFQNKAKVCWAKCLVQVLETAGIRMTAEEWDTVLAFREDRAYSPEVALNEICTKYYGVDLDSGLFSAQSVSLYYENNHWDNRPGGRMYGFNREVARKFEQRYPFLRGKMDSGLQVNVPERKVQPFNAECNILLLNRRLPHALVTSYQQCRGERVEWLLKKLPGYHLLLVSEYNLALPHKRVFWIAPPHVSGADRIYDLDLGLPLNAGRYDLVFVNIHTEYRTHHYQQCVDHSMKLQMLGGDSLHLLXPGGSLLIRAYGYADRVSEMVVTALARKFSAFRVLRPACVTSNTEVFLLFTNFDNGRRAVTLHQANQRLSSMFACNGLHTAGC

SEQ ID NO: 7 - protein, 799 aa, Semliki Forest virus
GVVETPRSALKVTAQPNDVLLGNYVVLSPQTVLKSSKLAPVHPLAEQVKIITHNGRAGRYQVDGYDGRVLLPCGSAIPVPEFQALSESATMVYNEREFVNRKLYHIAVHGPSLNTDEENYEKVRAERTDAEYVFDVDKKCCVKREEASGLVLVGELTNPPFHEFAYEGLKIRPSAPYKTTVVGVFGVPGSGKSAIIKSLVTKHDLVTSGKKENCQEIVNDVKKHRGLDIQAKTVDSILLNGCRRAVDILYVDEAFACHSGTLLALIALVKPRSKVVLCGDPKQCGFFNMMQLKVNFNHNICTEVCHKSISRRCTRPVTAIVSTLHYGGKMRTTNPCNKPIIIDTTGQTKPKPGDIVLTCFRGWVKQLQLDYRGHEVMTAAASQGLTRKGVYAVRQKVNENPLYAPASEHVNVLLTRTEDRLVWKTLAGDPWIKVLSNIPQGNFTATLEEWQEEHDKIMKVIEGPAAPVDAFQNKANVCWAKSLVPVLDTAGIRLTAEEWSTIITAFKEDRAYSPVVALNEICTKYYGVDLDSGLFSAPKVSLYYENNHWDNRPGGRMYGFNAATAARLEARHTFLKGQWHTGKQAVIAERKIQPLSVLDNVIPINRRLPHALVAEYKTVKGSRVEWLVNKVRGYHVLLVSEYNLALPRRRVTWLSPLNVTGADRCYDLSLGLPADAGRFDLVFVNIHTEFRIHHYQQCVDHAMKLQMLGGDALRLLKPGGSLLMRAYGYADKISEAVVSSLSRKFSSARVLRPDCVTSNTEVFLLFSNFDNGKRPSTLHQMNTKLSAVYAGEAMHTAGC

SEQ ID NO: 8 - protein, 798 aa, getah virus
GVVETPRNALKVTPQAHDHLIGSYLILSPQTVLKSEKLAPIHPLAEQVTVMTHSGRSGRYPVDKYDGRVLIPTGAAIPVSEFQALSESATMVYNEREFINRKLHHIALYGPALNTDEESYEKVRAERAETEYVFDVDKKACIKKEEASGLVLTGDLINPPFHEFAYEGLKIRPAAPYHTTIIGVFGVPGSGKSAIIKNMVTTRDLVASGKKENCQEIMNDVKRQRGLDVTARTVDSILLNGCKKGVENLYVDEAFACHSGTLLALIALVRPSGKVVLCGDPKQCGFFNLMQLKVHYNHNICTRVLHKSISRRCTLPVTAIVSTLHYQGKMRTTNRCNTPIQIDTTGSSKPASGDIVLTCFRGWVKQLQIDYRGHEVMTAAASQGLTRKGVYAVRQKVNENPLYSPLSEHVNVLLTRTENRLVWKTLSGDPWIKVLTNVPRGDFSATLEEWHEEHDGIMRVLNERPAEVDPFQNKAKVCWAKCLVQVLETAGIRMTADEWNTILAFREDRAYSPEVALNEICTRYYGVDLDSGLFSAQSVSLFYENNHWDNRPGGRMYGFNHEVARKYAARFPFLRGNMNSGLQLNVPERKLQPFSAECNIVPSNRRLPHALVTSYQQCRGERVEWLLKKIPGHQMLLVSEYNLVIPHKRVFWIAPPRVSGADRTYDLDLGLPMDAGRYDLVFVNIHTEYRQHHYQQCVDHSMRLQMLGGDSLHLLRPGGSLLMRAYGYADRVSEMVVTALARKFSAFRVLRPACVTSNTEVFLLFSNFDNGRRAVTLHQANQKLSSMYACNGLHTAGC

SEQ ID NO: 9 - protein, 798 aa, Chikungunya virus
GIIETPRGAIKVTAQLTDHVVGEYLVLSPQTVLRSQKLSLIHALAEQVKTCTHSGRAGRYAVEAYDGRVLVPSGYAISPEDFQSLSESATMVYNEREFVNRKLHHIAMHGPALNTDEESYELVRAERTEHEYVYDVDQRRCCKKEEAAGLVLVGDLTNPPYHEFAYEGLKIRPACPYKIAVIGVFGVPGSGKSAIIKNLVTRQDLVTSGKKENCQEISTDVMRQRGLEISARTVDSLLLNGCNRPVDVLYVDEAFACHSGTLLALIALVRPRQKVVLCGDPKQCGFFNMMQMKVNYNHNICTQVYHKSISRRCTLPVTAIVSSLHYEGKMRTTNEYNMPIVVDTTGSTKPDPGDLVLTCFRGWVKQLQIDYRGHEVMTAAASQGLTRKGVYAVRQKVNENPLYASTSEHVNVLLTRTEGKLVWKTLSGDPWIKTLQNPPKGNFKATIKEWEVEHASIMAGICSHQVTFDTFQNKANVCWAKSLVPILETAGIKLNDRQWSQIIQAFKEDKAYSPEVALNEICTRMYGVDLDSGLFSKPLVSVYYADNHWDNRPGGKMFGFNPEAASILERKYPFTKGKWNINKQICVTTRRIEDFNPTTNIIPVNRRLPHSLVAEHRPVKGERMEWLVNKINGHHVLLVSGYNLALPTKRVTWVAPLGVRGADYTYNLELGLPATLGRYDLVVINIHTPFRIHHYQQCVDHAMKLQMLGGDSLRLLKPGGSLLIRAYGYADRTSERVICVLGRKFRSSRALKPPCVTSNTEMFFLFSNFDNGRRNFTTHVMNNQLNAAFVGQATRAGC

SEQ ID NO: 10 - protein, 798 aa, O′nyong-nyong virus
GIVETPRGAIKVTAQPSDLVVGEYLVLTPQAVLRSQKLSLIHALAEQVKTCTHSGRAGRYAVEAYDGRVLVPSGYAIPQEDFQSLSESATMVFNEREFVNRKLHHIAMHGPALNTDEESYELVRVEKTEHEYVYDVDQKKCCKREEATGLVLVGDLTSPPYHEFAYEGLKIRPACPYKTAVIGVFGVPGSGKSAIIKNLVTRQDLVTSGKKENCQEISNDVMRQRKLEISARTVDSLLLNGCNKPVEVLYVDEAFACHSGTLLALIAMVRPRQKVVLCGDPKQCGFFNMMQMKVNYNHNICTQVYHKSISRRCTLPVTAIVSSLHYESKMRTTNEYNQPIVVDTTGITKPEPGDLVLTCFRGWVKQLQIDYRGNEVMTAAASQGLTRKGVYAVRQKVNENPLYAPTSEHVNVLLTRTEGKLTWKTLSGDPWIKILQNPPKGDFKATIKEWEAEHASIMAGICNHQMAFDTFQNKANVCWAKCLVPILDTAGIKLSDRQWSQIVQAFKEDRAYSPEVALNEICTRIYGVDLDSGLFSKPLISVYYADNHWDNRPGGKMFGFNPEVALMLEKKYPFTKGKWNINKQICITTRKVDEFNPETNIIPANRRLPHSLVAEHHSVRGERMEWLVNKISGHHMLLVSGHNLILPTKRVTWVAPLGTRGADYTYNLELGLPATLGRYDLVVINIHTPFRIHHYQQCVDHAMKLQMLGGDSLRLLKPGGSLLIRAYGYADRTSERVISVLGRKFRSSRALKPQCITSNTEMFFLFSRFDNGRRNFTTHVMNNQLNAVYAGLATRAGC

SEQ ID NO: 11 - protein, 46 aa, Artificial
  nsP3wt
APSYRTKRENIADCQEEAVVNAANPLGRPGEGVCRAIYKRWPTSFT

SEQ ID NO: 12 - protein, 40 aa, Artificial
  nsP3delta
APSYRTKRENIADCQEEAVVNAAGEGVCRAIYKRWPTSFT

SEQ ID NO: 13 - protein, 10 aa, Sindbis virus
FGFPPQARYD

SEQ ID NO: 14 - protein, 10 aa, Sindbis virus
FGFPPQSRYD

SEQ ID NO: 15 - protein, 10 aa, Aura virus
FGFPPLSRYD

SEQ ID NO: 16 - protein, 11 aa, Mayaro virus
LGLPPNAGRYD

SEQ ID NO: 17 - protein, 11 aa, Ross River virus
LGLPLNAGRYD

SEQ ID NO: 18 - protein, 11 aa, Semliki Forest virus
LGLPADAGRFD

SEQ ID NO: 19 - protein, 11 aa, Semliki Forest virus
LGLPADAGRYD

SEQ ID NO: 20 - protein, 11 aa, getah virus
LGLPMDAGRYD

SEQ ID NO: 21 - protein, 11 aa, Chikungunya virus
LGLPATLGRHD

SEQ ID NO: 22 - protein, 11 aa, Chikungunya virus
MGLPATLGRYD

SEQ ID NO: 23 - protein, 11 aa, Chikungunya virus
LGLPATLGRYD

SEQ ID NO: 24 - protein, 11 aa, O′nyong-nyong virus
LGLPATLGRYD

SEQ ID NO: 25 - protein, 30 aa, Artificial
  nsP2 CHIKV mutant
GADYTYNLELGLPATLGRYDLVVINIHTPF

SEQ ID NO: 26 - protein, 30 aa, Artificial
  nsP2 CHIKV mutant
GADYTYNLELGLPNGKGRYDLVVINIHTPF

SEQ ID NO: 27 - protein, 30 aa, Artificial
  nsP2 CHIKV mutant
GADYTYNLELGLPERRGRYDLVVINIHTPF

SEQ ID NO: 28 - protein, 30 aa, Artificial
  nsP2 CHIKV mutant
GADYTYNLELGLPRLEGRYDLVVINIHTPF